SEQUENCE LISTING (146 sequences)

SEQ ID NO: 1 - protein, 118 aa, Artificial Sequence
  12H10.G7-VH
EVQLQESGPELVKPGASVKMSCKASGYTFTRYVMHWVKQRPGQGLEWIGFINPYNDDTKYNEKFKGKATLTSDKSSSTAYMELSSLTSEDSAVYHCARWRQLGSLDSWGQGTTLTVSS

SEQ ID NO: 2 - protein, 111 aa, Artificial Sequence
  12H10.G7-VL
NIVLTQSPASLAVSLGQRATISCRASESVDTYGSSFVHWYQQKPGQPPKLLIYLASNLESGVPARFSGSGSRSDFTLTIDPVEADDAATYYCQQNNEEPWTFGGGTKLEIK

SEQ ID NO: 3 - protein, 249 aa, Artificial Sequence
  scFv of humanized 12H10.G7 GB87(VH-VL)
QVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSSGGGGSGGGGSGGGGSGGGGSDIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIK

SEQ ID NO: 4 - protein, 6 aa, Artificial Sequence
  12H10.G7-VH CDR2
NPYNDD

SEQ ID NO: 5 - protein, 9 aa, Artificial Sequence
  12H10.G7-VH CDR3
WRQLGSLDS

SEQ ID NO: 6 - protein, 15 aa, Artificial Sequence
  12H10.G7-VL CDR1
RASESVDTYGSSFVH

SEQ ID NO: 7 - protein, 7 aa, Artificial Sequence
  12H10.G7-VL CDR2
LASNLES

SEQ ID NO: 8 - protein, 9 aa, Artificial Sequence
  12H10.G7-VL CDR3
QQNNEEPWT

SEQ ID NO: 9 - protein, 118 aa, Artificial Sequence
  Humanized 12H10.G7-VH
QVQLVQSGAEVKKPGASVKVSCKASGYTFTRYVMHWVRQAPGQRLEWMGFINPYNDDTKYNEKFKGRVTITSDTSASTAYMELSSLRSEDTAVYHCARWRQLGSLDSWGQGTTVTVSS

SEQ ID NO: 10 - protein, 111 aa, Artificial Sequence
  Humanized 12H10.G7-VL
DIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGGGTKVEIK

SEQ ID NO: 11 - protein, 7 aa, Artificial Sequence
  Humanized 12H10.G7 GB87/GB95-VH CDR1
GYTFTRY

SEQ ID NO: 12 - protein, 249 aa, Artificial Sequence
  scFv of humanized 12H10.G7 GB95(VL-VH)
DIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSS

SEQ ID NO: 13 - protein, 118 aa, Artificial Sequence
  Humanized 12H10.G7 GB88/GB96-VH
QVQLVQSGAEVKKPGASVKVSCKASGYTFTRYVMHWVRQAPGQRLEWMGFINPYNDDTKYNEKFKGRVTITRDTSASTAYMELSSLRSEDTAVYHCARWRQLGSLDSWGQGTTVTVSS

SEQ ID NO: 14 - protein, 118 aa, Artificial Sequence
  Humanized 11F4.B9
QVQLVQSGAEVKKPGASVKVSCKASGYSFTGYYIHWVRQGPGQGLEWMGEIIPSTGSTIYAQKFQGRVTMTRDTSTSTVYMELSSLRSEDTAVYYCERWGDYYGRDYWGQGTLVTVSS

SEQ ID NO: 15 - protein, 249 aa, Artificial Sequence
  scFv of humanized 12H10.G7 GB88(VH-VL)
QVQLVQSGAEVKKPGASVKVSCKASGYTFTRYVMHWVRQAPGQCLEWMGFINPYNDDTKYNEKFKGRVTITRDTSASTAYMELSSLRSEDTAVYHCARWRQLGSLDSWGQGTTVTVSSGGGGSGGGGSGGGGSGGGGSDIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIK

SEQ ID NO: 16 - protein, 249 aa, Artificial Sequence
  scFv of humanized 12H10.G7 GB96(VL-VH)
DIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITRDTSASTAYMELSSLRSEDTAVYHCARWRQLGSLDSWGQGTTVTVSS

SEQ ID NO: 17 - protein, 118 aa, Artificial Sequence
  Humanized 12H10.G7 GB89/GB97-VH
QVQLVQSGAEVKKPGASVKVSCKASGYTFTRYVMHWVRQAPGQRLEWMGFINPYNDDTKYNEKFKGRVTITSDTSASTAYMELSSLRSEDTAVYYCARWRQLGSLDSWGQGTTVTVSS

SEQ ID NO: 18 - protein, 517 aa, Artificial Sequence
  extracellular region of hFLT3-ITD
NQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNIS

SEQ ID NO: 19 - protein, 249 aa, Artificial Sequence
  scFv of humanized 12H10.G7 GB89(VH-VL)
QVQLVQSGAEVKKPGASVKVSCKASGYTFTRYVMHWVRQAPGQCLEWMGFINPYNDDTKYNEKFKGRVTITSDTSASTAYMELSSLRSEDTAVYYCARWRQLGSLDSWGQGTTVTVSSGGGGSGGGGSGGGGSGGGGSDIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIK

SEQ ID NO: 20 - protein, 249 aa, Artificial Sequence
  scFv of humanized 12H10.G7 GB97(VL-VH)
DIVMTQSPASLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITSDTSASTAYMELSSLRSEDTAVYYCARWRQLGSLDSWGQGTTVTVSS

SEQ ID NO: 21 - protein, 226 aa, Homo sapiens
  wild-type human IgG1 Fc sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 111 aa, Artificial Sequence
  Humanized 12H10.G7 GB90/GB98-VL
DIVMTQSPDSLAVSLGERATINCRASESVDTYGSSFVHWYQQKPGQPPKLLIYLASNLESGVPDRFSGSGSRTDFTLTISSLQAEDAATYYCQQNNEEPWTFGGGTKVEIK

SEQ ID NO: 23 - protein, 249 aa, Artificial Sequence
  scFv of humanized 12H10.G7 GB90(VH-VL)
QVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSSGGGGSGGGGSGGGGSGGGGSDIVMTQSPDSLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIK

SEQ ID NO: 24 - protein, 249 aa, Artificial Sequence
  scFv of humanized 12H10.G7 GB98(VL-VH)
DIVMTQSPDSLAVSLGERATINCRASESVDTYGSSFVHWYQQKPGQPPKLLIYLASNLESGVPDRFSGSGSRTDFTLTISSLQAEDAATYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSS

SEQ ID NO: 25 - protein, 517 aa, Artificial Sequence
  extracellular region of hFLT3-T227M
NQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGMDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNIS

SEQ ID NO: 26 - protein, 111 aa, Artificial Sequence
  scFv of humanized 12H10.G7-VL
DIVMTQSPASLAVSLGERATINCRASESVDTYGSSFVHWYQQKPGQPPKLLIYLASNLESGVPDRFSGSGSGTDFTLTISSLQAEDAATYYCQQNNEEPWTFGGGTKVEIK

SEQ ID NO: 27 - protein, 249 aa, Artificial Sequence
  scFv of humanized 12H10.G7 GB91(VH-VL)
QVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSSGGGGSGGGGSGGGGSGGGGSDIVMTQSPASLAVSLGERATINCRASESVDTYGSSFVHWYQQKPGQPPKLLIYLASNLESGVPDRFSGSGSGTDFTLTISSLQAEDAATYYCQQNNEEPWTFGCGTKVEIK

SEQ ID NO: 28 - protein, 249 aa, Artificial Sequence
  scFv of humanized 12H10.G7 GB99(VL-VH)
DIVMTQSPASLAVSLGERATINCRASESVDTYGSSFVHWYQQKPGQPPKLLIYLASNLESGVPDRFSGSGSGTDFTLTISSLQAEDAATYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSS

SEQ ID NO: 29 - protein, 118 aa, Artificial Sequence
  Humanized 14A5.E8 consensus-VH
  misc_feature (30)..(30): Xaa = Phe or Thr
  misc_feature (31)..(31): Xaa = Ser or Tyr
  misc_feature (40)..(40): Xaa = Ala or Arg
  misc_feature (44)..(44): Xaa = Cys or Gly
  misc_feature (72)..(72): Xaa = Val or Glu
  misc_feature (76)..(76): Xaa = Ser or Tyr
  misc_feature (100)..(100): Xaa = Asn or Val
  misc_feature (104)..(104): Xaa = Thr or Tyr
QVQLVQSGAEVKKPGASVKVSCKVSGYTFXXYWINWVRQXPGKXLEWMGNIYPGSSIINYNENFKNRVTMTXDTSXDTAYMELSSLRSEDTAVYYCARRXVYLXFDYWGQGTLVTVSS

SEQ ID NO: 30 - protein, 111 aa, Artificial Sequence
  Humanized 12H10.G7 GB92/GB100-VL
DIVMTQSPASLAVSLGERATINCRASESVDTYGSSFVHWYQQKPGQPPKLLIYLASNLESGVPDRFSGSGSRTDFTLTISSLQAEDVATYYCQQNNEEPWTFGGGTKVEIK

SEQ ID NO: 31 - protein, 249 aa, Artificial Sequence
  scFv of humanized 12H10.G7 GB92(VH-VL)
QVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSSGGGGSGGGGSGGGGSGGGGSDIVMTQSPASLAVSLGERATINCRASESVDTYGSSFVHWYQQKPGQPPKLLIYLASNLESGVPDRFSGSGSRTDFTLTISSLQAEDVATYYCQQNNEEPWTFGCGTKVEIK

SEQ ID NO: 32 - protein, 247 aa, Artificial Sequence
  scFv of humanized 12H10.G7 GB100(VL-VH)
DIVMTQSPASLAVSLGERATINCRASESVDTYGSSFVHWYQQKPGQPPKLLIYLASNLESGVPDRFSGSGSRTDFTLTISSLQAEDVATYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTV

SEQ ID NO: 33 - protein, 6 aa, Artificial Sequence
  4H2.E3-VH CDR2
NPYSDG

SEQ ID NO: 34 - protein, 111 aa, Artificial Sequence
  Humanized 12H10.G7 GB93/GB101-VL
DIVMTQSPASLAVSLGERATINCRASESVDTYGSSFVHWYQQKPGQPPKLLIYLASNLESGVPDRFSGSGSRTDFTLTISSLQAEDAAVYYCQQNNEEPWTFGGGTKVEIK

SEQ ID NO: 35 - protein, 249 aa, Artificial Sequence
  scFv of humanized 12H10.G7 GB93(VH-VL)
QVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSSGGGGSGGGGSGGGGSGGGGSDIVMTQSPASLAVSLGERATINCRASESVDTYGSSFVHWYQQKPGQPPKLLIYLASNLESGVPDRFSGSGSRTDFTLTISSLQAEDAAVYYCQQNNEEPWTFGCGTKVEIK

SEQ ID NO: 36 - protein, 249 aa, Artificial Sequence
  scFv of humanized 12H10.G7 GB101(VL-VH)
DIVMTQSPASLAVSLGERATINCRASESVDTYGSSFVHWYQQKPGQPPKLLIYLASNLESGVPDRFSGSGSRTDFTLTISSLQAEDAAVYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITSDTSASTAYMELSSLRSEDTAVYHCARWRQLGSLDSWGQGTTVTVSS

SEQ ID NO: 37 - protein, 118 aa, Artificial Sequence
  Humanized 12H10.G7 GB94/GB102-VH
QVQLVQSGAEVKKPGASVKVSCKASGYTFTRYVMHWVRQAPGQRLEWMGFINPYNDDTKYNEKFKGRVTITRDTSASTAYMELSSLRSEDTAVYYCARWRQLGSLDSWGQGTTVTVSS

SEQ ID NO: 38 - protein, 111 aa, Artificial Sequence
  Humanized 12H10.G7 GB94/GB102-VL
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGGGTKVEIK

SEQ ID NO: 39 - protein, 249 aa, Artificial Sequence
  scFv of humanized 12H10.G7 GB94(VH-VL)
QVQLVQSGAEVKKPGASVKVSCKASGYTFTRYVMHWVRQAPGQCLEWMGFINPYNDDTKYNEKFKGRVTITRDTSASTAYMELSSLRSEDTAVYYCARWRQLGSLDSWGQGTTVTVSSGGGGSGGGGSGGGGSGGGGSDIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIK

SEQ ID NO: 40 - protein, 249 aa, Artificial Sequence
  scFv of humanized 12H10.G7 GB102(VL-VH)
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITRDTSASTAYMELSSLRSEDTAVYYCARWRQLGSLDSWGQGTTVTVSS

SEQ ID NO: 41 - protein, 118 aa, Artificial Sequence
  Humanized 12H10.G7 GB102 D101E-VH
QVQLVQSGAEVKKPGASVKVSCKASGYTFTRYVMHWVRQAPGQRLEWMGFINPYNDDTKYNEKFKGRVTITRDTSASTAYMELSSLRSEDTAVYYCARWRQLGSLESWGQGTTVTVSS

SEQ ID NO: 42 - protein, 111 aa, Artificial Sequence
  Humanized 12H10.G7 GB102 -VL
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIK

SEQ ID NO: 43 - protein, 249 aa, Artificial Sequence
  scFv of humanized 12H10.G7 GB102 D101E (VH-VL)
QVQLVQSGAEVKKPGASVKVSCKASGYTFTRYVMHWVRQAPGQCLEWMGFINPYNDDTKYNEKFKGRVTITRDTSASTAYMELSSLRSEDTAVYYCARWRQLGSLESWGQGTTVTVSSGGGGSGGGGSGGGGSGGGGSDIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIK

SEQ ID NO: 44 - protein, 249 aa, Artificial Sequence
  scFv of humanized 12H10.G7 GB102 D101E (VL-VH)
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIKGGGGSGGGGSGGGGSGGGGSQVQLVQSGAEVKKPGASVKVSCKASGYTFTRYVMHWVRQAPGQCLEWMGFINPYNDDTKYNEKFKGRVTITRDTSASTAYMELSSLRSEDTAVYYCARWRQLGSLESWGQGTTVTVSS

SEQ ID NO: 45 - protein, 118 aa, Artificial Sequence
  Humanized 12H10.G7 GB102 M34I-VH
QVQLVQSGAEVKKPGASVKVSCKASGYTFTRYVIHWVRQAPGQRLEWMGFINPYNDDTKYNEKFKGRVTITRDTSASTAYMELSSLRSEDTAVYYCARWRQLGSLDSWGQGTTVTVSS

SEQ ID NO: 46 - protein, 9 aa, Artificial Sequence
  14H8.E7-VL CDR3
QQWSSKSPT

SEQ ID NO: 47 - protein, 249 aa, Artificial Sequence
  scFv of humanized 12H10.G7 GB102 M34I (VH-VL)
QVQLVQSGAEVKKPGASVKVSCKASGYTFTRYVIHWVRQAPGQCLEWMGFINPYNDDTKYNEKFKGRVTITRDTSASTAYMELSSLRSEDTAVYYCARWRQLGSLDSWGQGTTVTVSSGGGGSGGGGSGGGGSGGGGSDIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIK

SEQ ID NO: 48 - protein, 249 aa, Artificial Sequence
  scFv of humanized 12H10.G7 GB102 M34I (VL-VH)
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIKGGGGSGGGGSGGGGSGGGGSQVQLVQSGAEVKKPGASVKVSCKASGYTFTRYVIHWVRQAPGQCLEWMGFINPYNDDTKYNEKFKGRVTITRDTSASTAYMELSSLRSEDTAVYYCARWRQLGSLDSWGQGTTVTVSS

SEQ ID NO: 49 - protein, 118 aa, Artificial Sequence
  Humanized 12H10.G7 GB102 M34I/D101E-VH
QVQLVQSGAEVKKPGASVKVSCKASGYTFTRYVIHWVRQAPGQRLEWMGFINPYNDDTKYNEKFKGRVTITRDTSASTAYMELSSLRSEDTAVYYCARWRQLGSLESWGQGTTVTVSS

SEQ ID NO: 50 - protein, 9 aa, Artificial Sequence
  Humanized 12H10.G7 GB102 M34I/D101E -CDR3
WRQLGSLES

SEQ ID NO: 51 - protein, 249 aa, Artificial Sequence
  scFv of humanized 12H10.G7 GB102 M34I/D101E (VH-VL)
QVQLVQSGAEVKKPGASVKVSCKASGYTFTRYVIHWVRQAPGQCLEWMGFINPYNDDTKYNEKFKGRVTITRDTSASTAYMELSSLRSEDTAVYYCARWRQLGSLESWGQGTTVTVSSGGGGSGGGGSGGGGSGGGGSDIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIK

SEQ ID NO: 52 - protein, 249 aa, Artificial Sequence
  scFv of humanized 12H10.G7 GB102 M34I/D101E (VL-VH)
DIVMTQSPDSLAVSLGERATINCRASESVDTYGSSFVHWYQQKPGQPPKLLIYLASNLESGVPDRFSGSGSGTDFTLTISSLQAEDVAVYYCQQNNEEPWTFGCGTKVEIKGGGGSGGGGSGGGGSGGGGSQVQLVQSGAEVKKPGASVKVSCKASGYTFTRYVIHWVRQAPGQCLEWMGFINPYNDDTKYNEKFKGRVTITRDTSASTAYMELSSLRSEDTAVYYCARWRQLGSLESWGQGTTVTVSS

SEQ ID NO: 53 - protein, 118 aa, Artificial Sequence
  Humanized 12H10.G7 consensus 1-VH
  misc_feature (34)..(34): Xaa = Met or Ile
  misc_feature (106)..(106): Xaa = Glu or Asp
QVQLVQSGAEVKKPGASVKVSCKASGYTFTRYVXHWVRQAPGQRLEWMGFINPYNDDTKYNEKFKGRVTITRDTSASTAYMELSSLRSEDTAVYYCARWRQLGSLXSWGQGTTVTVSS

SEQ ID NO: 54 - protein, 9 aa, Artificial Sequence
  Humanized 14A5.E8 consensus VH CDR3
  misc_feature (2)..(2): Xaa = Asn or Val
  misc_feature (6)..(6): Xaa = Thr or Tyr
RXVYLXFDY

SEQ ID NO: 55 - protein, 9 aa, Artificial Sequence
  Humanized 12H10.G7 consensus 1-VH CDR3
  misc_feature (8)..(8): Xaa = Glu or Asp
WRQLGSLXS

SEQ ID NO: 56 - protein, 118 aa, Artificial Sequence
  Humanized 12H10.G7 consensus 2 -VH
  misc_feature (72)..(72): Xaa = Ser or Arg
  misc_feature (95)..(95): Xaa = Tyr or His
QVQLVQSGAEVKKPGASVKVSCKASGYTFTRYVMHWVRQAPGQRLEWMGFINPYNDDTKYNEKFKGRVTITXDTSASTAYMELSSLRSEDTAVYXCARWRQLGSLDSWGQGTTVTVSS

SEQ ID NO: 57 - protein, 111 aa, Artificial Sequence
  Humanized 12H10.G7 consensus 2 -VL
  misc_feature (9)..(9): Xaa = Ala or Asp
  misc_feature (72)..(72): Xaa = Arg or Gly
  misc_feature (87)..(87): Xaa = Ala or Val
  misc_feature (89)..(89): Xaa = Thr or Val
DIVMTQSPXSLAVSLGERATINCRASESVDTYGSSFVHWYQQKPGQPPKLLIYLASNLESGVPDRFSGSGSXTDFTLTISSLQAEDXAXYYCQQNNEEPWTFGGGTKVEIK

SEQ ID NO: 58 - protein, 118 aa, Artificial Sequence
  Humanized 12H10.G7 consensus 3-VH
  misc_feature (34)..(34): Xaa = Met or Ile
  misc_feature (106)..(106): Xaa = Glu or Asp
QVQLVQSGAEVKKPGASVKVSCKASGYTFTRYVXHWVRQAPGQCLEWMGFINPYNDDTKYNEKFKGRVTITRDTSASTAYMELSSLRSEDTAVYYCARWRQLGSLXSWGQGTTVTVSS

SEQ ID NO: 59 - protein, 7 aa, Artificial Sequence
  Humanized 14A5.E8 consensus VH CDR1
  misc_feature (5)..(5): Xaa = Pro or Thr
  misc_feature (6)..(6): Xaa = Ser or Tyr
GYTFXXY

SEQ ID NO: 60 - protein, 118 aa, Artificial Sequence
  14A5.E8-VH
EVQLQESGAELVQPGASVRLSCKASGYTFTSYWINWVKQRPGQGLEWIGNIYPGSSIINYNENFKNRATLTVDTSSSTAYMQLSSLTSDDSAVYYCARRVVYLYFDYWGQGTTLTVSS

SEQ ID NO: 61 - protein, 106 aa, Artificial Sequence
  14A5.E8-VL
QIVLTQSPAIMSASPGEKVTMTCSASSSVSYMHWYQQKSGTSPKRWIYDTSKLASGVPARFSGSGSGTSYSLTISSMEAEDAATYYCQQWTSKSPTFGGGTKLEIK

SEQ ID NO: 62 - protein, 7 aa, Artificial Sequence
  14A5.E8-VH CDR1
GYTFTSY

SEQ ID NO: 63 - protein, 6 aa, Artificial Sequence
  14A5.E8-VH or Humanized 14A5.E8 consensus VH CDR2
YPGSSI

SEQ ID NO: 64 - protein, 9 aa, Artificial Sequence
  14A5.E8-VH CDR3
RVVYLYFDY

SEQ ID NO: 65 - protein, 10 aa, Artificial Sequence
  14A5.E8-VL CDR1
SASSSVSYMH

SEQ ID NO: 66 - protein, 7 aa, Artificial Sequence
  14A5.E8-VL or Humanized 14A5.E8 consensus VL CDR2
DTSKLAS

SEQ ID NO: 67 - protein, 9 aa, Artificial Sequence
  14A5.E8-VL or Humanized 14A5.E8 consensus VL CDR3
QQWTSKSPT

SEQ ID NO: 68 - protein, 118 aa, Artificial Sequence
  Humanized 14A5.E8 1551/1552-VH
QVQLVQSGAEVKKPGASVKVSCKVSGYTFTSYWINWVRQRPGKGLEWMGNIYPGSSIINYNENFKNRVTMTVDTSSDTAYMELSSLRSEDTAVYYCARRVVYLYFDYWGQGTLVTVSS

SEQ ID NO: 69 - protein, 106 aa, Artificial Sequence
  Humanized 14A5.E8 1551/1552-VL
EIVLTQSPATLSLSPGEKATLSCSASSSVSYMHWYQQKPGQAPRLLIYDTSKLASGIPARFSGSGSGTSFTLTISSLEPEDAAVYYCQQWTSKSPTFGGGTKVEIK

SEQ ID NO: 70 - protein, 244 aa, Artificial Sequence
  scFv of humanized 14A5.E8 1551(VH-VL)
QVQLVQSGAEVKKPGASVKVSCKVSGYTFTSYWINWVRQRPGKCLEWMGNIYPGSSIINYNENFKNRVTMTVDTSSDTAYMELSSLRSEDTAVYYCARRVVYLYFDYWGQGTLVTVSSGGGGSGGGGSGGGGSGGGGSEIVLTQSPATLSLSPGEKATLSCSASSSVSYMHWYQQKPGQAPRLLIYDTSKLASGIPARFSGSGSGTSFTLTISSLEPEDAAVYYCQQWTSKSPTFGCGTKVEIK

SEQ ID NO: 71 - protein, 244 aa, Artificial Sequence
  scFv of humanized 14A5.E8 1552(VL-VH)
EIVLTQSPATLSLSPGEKATLSCSASSSVSYMHWYQQKPGQAPRLLIYDTSKLASGIPARFSGSGSGTSFTLTISSLEPEDAAVYYCQQWTSKSPTFGCGTKVEIKGGGGSGGGGSGGGGSGGGGSQVQLVQSGAEVKKPGASVKVSCKVSGYTFTSYWINWVRQRPGKCLEWMGNIYPGSSIINYNENFKNRVTMTVDTSSDTAYMELSSLRSEDTAVYYCARRVVYLYFDYWGQGTLVTVSS

SEQ ID NO: 72 - protein, 118 aa, Artificial Sequence
  Humanized 14A5.E8 1553/1554-VH
QVQLVQSGAEVKKPGASVKVSCKVSGYTFTSYWINWVRQAPGKGLEWMGNIYPGSSIINYNENFKNRVTMTEDTSTDTAYMELSSLRSEDTAVYYCARRVVYLYFDYWGQGTLVTVSS

SEQ ID NO: 73 - protein, 106 aa, Artificial Sequence
  Humanized 14A5.E8 1553/1554-VL
EIVLTQSPATLSLSPGERATLSCSASSSVSYMHWYQQKPGQAPRLLIYDTSKLASGIPARFSGSGSGTDFTLTISSLEPEDFAVYYCQQWTSKSPTFGGGTKVEIK

SEQ ID NO: 74 - protein, 244 aa, Artificial Sequence
  scFv of humanized 14A5.E8 1553(VH-VL)
QVQLVQSGAEVKKPGASVKVSCKVSGYTFTSYWINWVRQAPGKCLEWMGNIYPGSSIINYNENFKNRVTMTEDTSTDTAYMELSSLRSEDTAVYYCARRVVYLYFDYWGQGTLVTVSSGGGGSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCQQWTSKSPTFGCGTKVEIK

SEQ ID NO: 75 - protein, 244 aa, Artificial Sequence
  scFv of humanized 14A5.E8 1554(VL-VH)
EIVLTQSPATLSLSPGERATLSCSASSSVSYMHWYQQKPGQAPRLLIYDTSKLASGIPARFSGSGSGTDFTLTISSLEPEDFAVYYCQQWTSKSPTFGCGTKVEIKGGGGSGGGGSGGGGSGGGGSQVQLVQSGAEVKKPGASVKVSCKVSGYTFTSYWINWVRQAPGKCLEWMGNIYPGSSIINYNENFKNRVTMTEDTSTDTAYMELSSLRSEDTAVYYCARRVVYLYFDYWGQGTLVTVSS

SEQ ID NO: 76 - protein, 118 aa, Artificial Sequence
  Humanized 14A5.E8 1689-VH
QVQLVQSGAEVKKPGASVKVSCKVSGYTFPYYWINWVRQAPGKGLEWMGNIYPGSSIINYNENFKNRVTMTEDTSTDTAYMELSSLRSEDTAVYYCARRNVYLTFDYWGQGTLVTVSS

SEQ ID NO: 77 - protein, 106 aa, Artificial Sequence
  Humanized 14A5.E8 1689-VL
EIVLTQSPATLSLSPGERATLSCSASSSVSYIHWYQQKPGQAPRLLIYDTSKLASGIPARFSGSGSGTDFTLTISSLEPEDFAVYYCQQWTSKSPTFGGGTKVEIK

SEQ ID NO: 78 - protein, 7 aa, Artificial Sequence
  Humanized 14A5.E8 1689-VH CDR1
GYTFPYY

SEQ ID NO: 79 - protein, 9 aa, Artificial Sequence
  Humanized 14A5.E8 1689-VH CDR3
RNVYLTFDY

SEQ ID NO: 80 - protein, 10 aa, Artificial Sequence
  Humanized 14A5.E8 1689-VL CDR1
SASSSVSYIH

SEQ ID NO: 81 - protein, 244 aa, Artificial Sequence
  scFv of humanized 14A5.E8 1689 (VH-VL)
QVQLVQSGAEVKKPGASVKVSCKVSGYTFPYYWINWVRQAPGKCLEWMGNIYPGSSIINYNENFKNRVTMTEDTSTDTAYMELSSLRSEDTAVYYCARRNVYLTFDYWGQGTLVTVSSGGGGSGGGGSGGGGSGGGGSEIVLTQSPATLSLSPGERATLSCSASSSVSYIHWYQQKPGQAPRLLIYDTSKLASGIPARFSGSGSGTDFTLTISSLEPEDFAVYYCQQWTSKSPTFGCGTKVEIK

SEQ ID NO: 82 - protein, 244 aa, Artificial Sequence
  scFv of humanized 14A5.E8 1689 (VL-VH)
EIVLTQSPATLSLSPGERATLSCSASSSVSYIHWYQQKPGQAPRLLIYDTSKLASGIPARFSGSGSGTDFTLTISSLEPEDFAVYYCQQWTSKSPTFGCGTKVEIKGGGGSGGGGSGGGGSGGGGSQVQLVQSGAEVKKPGASVKVSCKVSGYTFPYYWINWVRQAPGKCLEWMGNIYPGSSIINYNENFKNRVTMTEDTSTDTAYMELSSLRSEDTAVYYCARRNVYLTFDYWGQGTLVTVSS

SEQ ID NO: 83 - protein, 106 aa, Artificial Sequence
  14H8.E7-VL
QIVLTQSPAIMSASPGEKVTMTCSASSSVSYMHWYQQKSGTSPKRWIFDTSKLASGVPVRFSGSGSGTSYSLTITNMETEDAATYYCQQWSSKSPTFGGGTKLEIK

SEQ ID NO: 84 - protein, 106 aa, Artificial Sequence
  Humanized 14A5.E8 consensus VL
  misc_feature (32)..(32): Xaa = Met or Ile
EIVLTQSPATLSLSPGERATLSCSASSSVSYXHWYQQKPGQAPRLLIYDTSKLASGIPARFSGSGSGTDFTLTISSLEPEDFAVYYCQQWTSKSPTFGGGTKVEIK

SEQ ID NO: 85 - protein, 118 aa, Artificial Sequence
  11F4.B9-VH
EVQLQESGPELVKPGASVKISCKASGYSFTGYYIHWVKQGPEKSLEWIGEIIPSTGSTIYNQKFKAKATLTVDKSSSTAYLQLKSLTSEDSAVYYCERWGDYYGRDYWGQGTSVTVSS

SEQ ID NO: 86 - protein, 10 aa, Artificial Sequence
  Humanized 14A5.E8 consensus VL CDR1
  misc_feature (9)..(9): Xaa = Met or Ile
SASSSVSYXH

SEQ ID NO: 87 - protein, 7 aa, Artificial Sequence
  11F4.B9-VH CDR1
GYSFTGY

SEQ ID NO: 88 - protein, 6 aa, Artificial Sequence
  11F4.B9-VH CDR2
IPSTGS

SEQ ID NO: 89 - protein, 9 aa, Artificial Sequence
  11F4.B9-VH CDR3
WGDYYGRDY

SEQ ID NO: 90 - protein, 111 aa, Artificial Sequence
  11F4.B9-VL
DIVLTQSPASLAVSLGQRATISCRASESVDIYGNSFMHWYQQKPGQPPKLLIYRASNLESGIPARFSGSGSRTDFTLTINPVEADDVATYYCQQSNEDPRTFGGGTKLEIK

SEQ ID NO: 91 - protein, 15 aa, Artificial Sequence
  11F4.B9-VL CDR1
RASESVDIYGNSFMH

SEQ ID NO: 92 - protein, 7 aa, Artificial Sequence
  11F4.B9-VL CDR2
RASNLES

SEQ ID NO: 93 - protein, 9 aa, Artificial Sequence
  11F4.B9-VL CDR3
QQSNEDPRT

SEQ ID NO: 94 - protein, 111 aa, Artificial Sequence
  Humanized 11F4.B9-VL
DIVMTQSPASLAVSLGERATINCRASESVDIYGNSFMHWYQQKPGQPPKLLIYRASNLESGVPDRFSGSGSRTDFTLTINSLQAEDVATYYCQQSNEDPRTFGGGTKVEIK

SEQ ID NO: 95 - protein, 120 aa, Artificial Sequence
  4A4.A3-VH
QVTLKESGPGILQPSQTLSLTCSFSGFSLTTYGMGVGWIRQPSGKGLEWLANIWFNDNKYYNSTLKSRLTISKDTSNNQVFLKISSVDTTDTATYYCAQITTVVGTFDYWGQGSPLTVSP

SEQ ID NO: 96 - protein, 108 aa, Artificial Sequence
  4A4.A3-VL
RIVMTQSPTTMAASPGEKITITCSASSSISSIYLHWYQQKPGFSPKLLIFRTSDLASGVPPRFGGSGSGTSYSLTIGTMEAEDVATYYCQQGSSFPRTFGGGTKLEIK

SEQ ID NO: 97 - protein, 9 aa, Artificial Sequence
  4A4.A3-VH CDR1
GFSLTTYGM

SEQ ID NO: 98 - protein, 6 aa, Artificial Sequence
  4A4.H7-VH CDR2
YPNTGI

SEQ ID NO: 99 - protein, 5 aa, Artificial Sequence
  4A4.A3-VH CDR2
WFNDN

SEQ ID NO: 100 - protein, 10 aa, Artificial Sequence
  4A4.A3-VH CDR3
ITTVVGTFDY

SEQ ID NO: 101 - protein, 12 aa, Artificial Sequence
  4A4.A3-VL CDR1
SASSSISSIYLH

SEQ ID NO: 102 - protein, 7 aa, Artificial Sequence
  4A4.A3-VL CDR2
RTSDLAS

SEQ ID NO: 103 - protein, 9 aa, Artificial Sequence
  4A4.A3-VL CDR3
QQGSSFPRT

SEQ ID NO: 104 - protein, 118 aa, Artificial Sequence
  4A4.H7-VH
EVQLQESGPELVKPGASVKISCKASGYSFTGYYIHWVKQSPEESLEWIGEIYPNTGITTYNQKFTAKATLTVDKSSNTAYMQLKSLTSEDSAVYYCTRWGDYYGRDYWGQGTSVTVSS

SEQ ID NO: 105 - protein, 111 aa, Artificial Sequence
  4A4.H7-VL
DIVLTQSPASLAVSLGQRATISCRASETVDTHGNSFMHWYQQKPGQPPKLLIYRASNLESGIPARFSGSGSRTDFTLTINPVEADDVATYYCQQSNEDPRTFGGGTKLEIK

SEQ ID NO: 106 - protein, 15 aa, Artificial Sequence
  4A4.H7-VL CDR1
RASETVDTHGNSFMH

SEQ ID NO: 107 - protein, 118 aa, Artificial Sequence
  15A11.C8-VH
EVQLQESGGGLVKTGGSRKLSCAASGFTFSDYGMHWVRHTPEKGLEWVVYISSGGNTIFYTDTVKGRFTISRDNAKNTLFLQMTSLRSEDTAVYFCVRQGYYYAMDYWGQGASVTVSS

SEQ ID NO: 108 - protein, 107 aa, Artificial Sequence
  15A11.C8-VL
DIQMTQTTSSLSASLGDRVTIRCRASQDITNYLNWYQQKPDGAVKLLISYTSILQSGVPSRFSGSGSGTDYSLTISNLEQGDVATYFCQQGSSLPWTFGGGTKLEIK

SEQ ID NO: 109 - protein, 7 aa, Artificial Sequence
  15A11.C8-VH CDR1
GFTFSDY

SEQ ID NO: 110 - protein, 6 aa, Artificial Sequence
  15A11.C8-VH CDR2
SSGGNT

SEQ ID NO: 111 - protein, 9 aa, Artificial Sequence
  15A11.C8-VH CDR3
QGYYYAMDY

SEQ ID NO: 112 - protein, 11 aa, Artificial Sequence
  15A11.C8-VL CDR1
RASQDITNYLN

SEQ ID NO: 113 - protein, 7 aa, Artificial Sequence
  15A11.C8-VL CDR2
YTSILQS

SEQ ID NO: 114 - protein, 9 aa, Artificial Sequence
  15A11.C8-VL CDR3
QQGSSLPWT

SEQ ID NO: 115 - protein, 114 aa, Artificial Sequence
  12C9.E5-VH
EVQLQESGAELVRPGASVKLSCKASGYIFTDYEIHWVKQTPVHGLEWIGAIDPETGITAYSQKFKGKATLTTDTSSSTAYMEFRSLTSEDSAVYYCTRGGLLYWGQGTSVTVSS

SEQ ID NO: 116 - protein, 112 aa, Artificial Sequence
  12C9.E5-VL
DVVMTQTPLSLSVTIGQPASISCKSSQSLLYSDGETYLNWLQQRPGQSPKRLMYQVSKLDPGIPDRFSGSGSETDFTLKISRVEAEDLGIYYCLQGTFYPHTFGGGTKLEIK

SEQ ID NO: 117 - protein, 7 aa, Artificial Sequence
  12C9.E5-VH CDR1
GYIFTDY

SEQ ID NO: 118 - protein, 6 aa, Artificial Sequence
  12C9.E5-VH CDR2
DPETGI

SEQ ID NO: 119 - protein, 5 aa, Artificial Sequence
  12C9.E5-VH CDR3
GGLLY

SEQ ID NO: 120 - protein, 16 aa, Artificial Sequence
  12C9.E5-VL CDR1
KSSQSLLYSDGETYLN

SEQ ID NO: 121 - protein, 7 aa, Artificial Sequence
  12C9.E5-VL CDR2
QVSKLDP

SEQ ID NO: 122 - protein, 9 aa, Artificial Sequence
  12C9.E5-VL CDR3
LQGTFYPHT

SEQ ID NO: 123 - protein, 118 aa, Artificial Sequence; identical to SEQ ID NO: 104
  1A2.A3 VH

SEQ ID NO: 124 - protein, 111 aa, Artificial Sequence; identical to SEQ ID NO: 105
  1A2.A3-VL

SEQ ID NO: 125 - protein, 120 aa, Artificial Sequence
  4H2.E3-VH
EVQLQESGPELVKPGASVKMSCKASGYTFTSYLMHWMKQKPGQGLEWIGYINPYSDGIKYNEKFRDKATLTSDKSSNTAYMELSSLTSEDSAVYYCAHSSGYVGYAMDYWGQGTSVTVSS

SEQ ID NO: 126 - protein, 112 aa, Artificial Sequence
  4H2.E3-VL
GIVMTQTTPSVPVTPGESVSISCRSSKSLLHSNGNTYLYWFLQRPGQSPQLLIYRMSNLASGVPDRFSGSGSGTTFTLRISRVEAEDVGVYYCMQHLEYPFTFGSGTKLEIK

SEQ ID NO: 127 - protein, 11 aa, Artificial Sequence
  4H2.E3-VH CDR3
SSGYVGYAMDY

SEQ ID NO: 128 - protein, 16 aa, Artificial Sequence
  4H2.E3-VL CDR1
RSSKSLLHSNGNTYLY

SEQ ID NO: 129 - protein, 7 aa, Artificial Sequence
  4H2.E3-VL CDR2
RMSNLAS

SEQ ID NO: 130 - protein, 9 aa, Artificial Sequence
  4H2.E3-VL CDR3
MQHLEYPFT

SEQ ID NO: 131 - protein, 118 aa, Artificial Sequence
  14H8.E7-VH
EVQLQESGAELVKPGASVKLSCKASGYTFTNYWINWLKQRPGQGLEWIGNIYPGSTIINYNEKFKNKATLTVDTSSSTAYMQLSSLTSDDSAVYYCARRVVYLYFDSWGQGTTLTVSS

SEQ ID NO: 132 - protein, 7 aa, Artificial Sequence
  14H8.E7-VH CDR1
GYTFTNY

SEQ ID NO: 133 - protein, 6 aa, Artificial Sequence
  14H8.E7-VH CDR2
YPGSTI

SEQ ID NO: 134 - protein, 9 aa, Artificial Sequence
  14H8.E7-VH CDR3
RVVYLYFDS

SEQ ID NO: 135 - protein, 118 aa, Artificial Sequence
  Alpha-FLT3 mAb 4G8-Synimmune VH
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSS

SEQ ID NO: 136 - protein, 107 aa, Artificial Sequence
  Alpha-FLT3 mAb 4G8-Synimmune VL
DIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIK

SEQ ID NO: 137 - protein, 119 aa, Artificial Sequence
  Alpha-FLT3 mAb EB10-ImClone&Lilly VH
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGVGAHDAFDIWGQGTTVTVSS

SEQ ID NO: 138 - protein, 112 aa, Artificial Sequence
  Alpha-FLT3 mAb EB10-ImClone&Lilly VL
DVVMTQSPLSLPVTPGEPASISCRSSQSLLHSNGNNYLDWYLQKPGQSPQLLIYLGSNRASGVPDRFSGSGSDTDFTLQISRVEAEDVGVYYCMQGTHPAISFGQGTRLEIK

SEQ ID NO: 139 - protein, 122 aa, Artificial Sequence
  Alpha-FLT3 mAb NC7 ImClone&Lilly VH
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSS

SEQ ID NO: 140 - protein, 107 aa, Artificial Sequence
  Alpha-FLT3 mAb NC7 ImClone&Lilly VL
DIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIK

SEQ ID NO: 141 - protein, 124 aa, Artificial Sequence
  Alpha-FLT3 mAb FL23(Amgen) VH
QVTLKESGPALVKPTETLTLTCTVSGFSFRNARMGVSWIRQPPGKALEWLAHIFSNDEKSYSTSLKSRLTISKDTSKSQVVLTLTNMDPVDTATYFCARMPEYSSGWSGAFDIWGQGTMVTVSS

SEQ ID NO: 142 - protein, 107 aa, Artificial Sequence
  Alpha-FLT3 mAb FL23(Amgen) VL
DIQMTQSPSSLSASVGDRVTITCRASQDIGYDLGWYQQKPGKAPKRLIYAASTLQSGVPSRFSGSGSGTEFTLIISSLQPEDFATYYCLQHNSFPWTFGQGTKVEIK

SEQ ID NO: 143 - protein, 124 aa, Artificial Sequence
  Alpha-FLT3 mAb FL39(Amgen) VH
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 144 - protein, 107 aa, Artificial Sequence
  Alpha-FLT3 mAb FL39(Amgen) VL
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 145 - protein, 129 aa, Artificial Sequence
  Alpha-FLT3 mAb FL61(Amgen) VH
QVQLVESGGGVVQPGRSLRLSCAASGFTFSSYGMHWVRQAPGKGLEWVAVISYDGSNEFYADSVKGRFTISRDNSKNTLYLQMNSLRAEDTAVYYCARGGEITMVRGVIGYYYYGMDVWGQGTTVTVSS

SEQ ID NO: 146 - protein, 107 aa, Artificial Sequence
  Alpha-FLT3 mAb FL61(Amgen) VL
DIQMTQSPSSLSASVGDRVTITCRASQSISSYLNWYQQKPGKAPKLLIYAASSLQSGVPSRFSGSGSGTEFTLTISSLQPEDFATYYCLQHNSYPLTFGGGTKVEIK

(19) United States
(12) Patent Application Publication
(10) Pub. No.: US 2024/0132598 A1
(43) Pub. Date: Apr. 25, 2024

(54) ANTIBODIES TARGETING FLT3 AND USE THEREOF

(71) Applicant: Dragonfly Therapeutics, Inc., Waltham, MA (US)

(72) Inventors: Hemanta Baruah, Euless, TX (US); Gregory P. Chang, Medford, MA (US); Ann F. Cheung, Lincoln, MA (US); Asya Grinberg, Lexington, MA (US); Zong Sean Juo, Cambridge, MA (US); Thomas J. McQuade, Boston, MA (US)

(21) Appl. No.: 17/769,257

(22) PCT Filed: Oct. 13, 2020
(86) PCT No.: PCT/US2020/055480

§ 371 Date: Apr. 13, 2022

Related U.S. Application Data
(60) Provisional application No. 62/915,120, filed on Oct. 15, 2019.

(51) Int. Cl. C07K 16/28; A61P 35/02

(52) U.S. Cl. CPC C07K 16/2863; A61P 35/02; C07K 2317/24; C07K 2317/31; C07K 2317/33; C07K 2317/565; C07K 2317/622; C07K 2317/73; C07K 2317/76; C07K 2317/77; C07K 2317/92

(57) ABSTRACT

Disclosed are proteins with antibody heavy chain and light chain variable domains that can be paired to form an antigen-binding site targeting FLT3 on a cell, pharmaceutical compositions comprising such proteins, and therapeutic methods using such proteins and pharmaceutical compositions, including for the treatment of cancer.

This application claims priority to U.S. Provisional Application No. 62/915,120, filed on Oct. 15, 2019, the entirety of which is incorporated herein by reference.

### SEQUENCE LISTING

This application incorporates by reference in its entirety the Computer Readable Form (CRF) of a Sequence Listing in ASCII text format. The Sequence Listing text file is entitled “14247-473-888_SEQ_LISTING,” was created on Oct. 5, 2020, and is 152,327 bytes in size.

### FIELD OF THE INVENTION

The invention provides proteins with antibody heavy chain and light chain variable domains that can be paired to form an antigen-binding site targeting FLT3 on a cell, pharmaceutical compositions comprising such proteins, and therapeutic methods using such proteins and pharmaceutical compositions, including for the treatment of cancer.

### BACKGROUND

Cancer continues to be a significant health problem despite the substantial research efforts and scientific advances reported in the literature for treating this disease. Some of the most frequently diagnosed cancers in adults include prostate cancer, breast cancer, and lung cancer. Hematological malignancies, though less frequent than solid cancers, have low survival rates. Current treatment options for these cancers are not effective for all patients and/or can have substantial adverse side effects. Other types of cancer also remain challenging to treat using existing therapeutic options.

Fms related tyrosine kinase 3 (FLT3), also called FLK2, STK1, or CD135, is a class III receptor tyrosine kinase. FLT3 is a transmembrane protein including five immunoglobulin-like domains in the extracellular region. FLT3 can be activated by binding of FLT3LG, which induces FLT3 homodimerization and autophosphorylation. Activated FLT3 subsequently phosphorylates and activates multiple cytoplasmic effector molecules such as Akt, Erk, and mTOR, thereby promoting cell proliferation and reducing apoptosis. Mutations that result in constitutive activation of FLT3 have been observed in acute myeloid leukemia and acute lymphoblastic leukemia.

Although antibodies that bind FLT3 are under development, there still remains a need in the field for new and useful treatments for FLT3-related cancer.

### SUMMARY OF THE INVENTION

The present invention provides antigen-binding sites that bind human FLT3 and optionally bind cynomolgus FLT3. These antigen-binding sites bind various epitopes in an extracellular domain of FLT3, and some of them do not compete with FLT3-ligand (FLT3L) for such binding. Some of the antigen-binding sites disclosed herein bind unique epitopes compared to the epitopes targeted by one or more known anti-FLT3 antibodies in the art. Proteins and protein conjugates containing such antigen-binding sites, for example, antibodies, antibody-drug conjugates, bispecific T-cell engagers (BiTEs), and immunocytokines, as well as immune effector cells (e.g., T cells) expressing a protein containing such an antigen-binding site (e.g., a chimeric antigen receptor (CAR)), are useful for treating FLT3-associated diseases such as cancer.

Accordingly, in one aspect, the present invention provides an antigen-binding site that binds FLT3, comprising:
(a) a heavy chain variable domain (VH) comprising complementarity-determining region 1 (CDR1), complementarity-determining region 2 (CDR2), and complementarity-determining region 3 (CDR3) comprising the amino acid sequences of SEQ ID NOs: 11, 4, and 55, respectively; and
(b) a light chain variable domain (VL) comprising CDR1, CDR2, and CDR3 comprising the amino acid sequences of SEQ ID NOs: 6, 7, and 8, respectively.

In certain embodiments, the CDR3 of the VH comprises the amino acid sequence of SEQ ID NO:5. In certain embodiments, the CDR3 of the VH comprises the amino acid sequence of SEQ ID NO:50. In certain embodiments, the VH comprises an amino acid sequence at least 90% identical to SEQ ID NO:37, and the VL comprises an amino acid sequence at least 90% identical to SEQ ID NO:38. In certain embodiments, the VH comprises the amino acid sequence of SEQ ID NO:53, and the VL comprises the amino acid sequence of SEQ ID NO:42. In certain embodiments, the VH and the VL comprise the amino acid sequences of SEQ ID NOs: 9 and 10; 13 and 10; 17 and 10; 9 and 22; 9 and 26; 9 and 30; 9 and 34; 37 and 38; 41 and 42; 45 and 42; or 49 and 42, respectively.

In another aspect, the present invention provides an antigen-binding site that binds FLT3, comprising:
(a) a VH comprising CDR1, CDR2, and CDR3 comprising the amino acid sequences of SEQ ID NOs: 59, 63, and 54, respectively; and
(b) a VL comprising CDR1, CDR2, and CDR3 comprising the amino acid sequences of SEQ ID NOs: 86, 66, and 67, respectively.

In certain embodiments, the VH comprises CDR1, CDR2, and CDR3 of SEQ ID NO: 78, 63, 79, respectively, and the VL comprises CDR1, CDR2, and CDR3 of SEQ ID NO: 80, 66, 67, respectively. In certain embodiments, the VH comprises CDR1, CDR2, and CDR3 of SEQ ID NO: 62, 63, 64, respectively, and the VL comprises CDR1, CDR2, and CDR3 of SEQ ID NO: 65, 66, 67. In certain embodiments, the VH comprises an amino acid sequence at least 90% identical to SEQ ID NO:76, and the VL comprises an amino acid sequence at least 90% identical to SEQ ID NO:77. In certain embodiments, the VH comprises the amino acid sequence of SEQ ID NO:29, and the VL comprises the amino acid sequence of SEQ ID NO:84. In certain embodiments, the VH and the VL comprise the amino acid sequences of SEQ ID NOs: 68 and 69; 72 and 73; or 76 and 77, respectively.

In another aspect, the present invention provides an antigen-binding site that binds FLT3, comprising:
(a) a VH comprising CDR1, CDR2, and CDR3 comprising the amino acid sequences of SEQ ID NOs: 87, 88, and 89, respectively; and
(b) a VL comprising CDR1, CDR2, and CDR3 comprising the amino acid sequences of SEQ ID NOs: 91, 92, and 93, respectively.

In another aspect, the present invention provides an antigen-binding site that binds FLT3, comprising:
(a) a VH comprising CDR1, CDR2, and CDR3 comprising the amino acid sequences of SEQ ID NOs: 97, 99, and 100, respectively; and
(b) a VL comprising CDR1, CDR2, and CDR3 comprising the amino acid sequences of SEQ ID NOs: 101, 102, and 103, respectively.

In another aspect, the present invention provides an antigen-binding site that binds FLT3, comprising:
(a) a VH comprising CDR1, CDR2, and CDR3 comprising the amino acid sequences of SEQ ID NOs: 87, 98, and 89 respectively; and
(b) a VL comprising CDR1, CDR2, and CDR3 comprising the amino acid sequences of SEQ ID NOs: 106, 92, and 93, respectively.

In another aspect, the present invention provides an antigen-binding site that binds FLT3, comprising:
(a) a VH comprising CDR1, CDR2, and CDR3 comprising the amino acid sequences of SEQ ID NOs: 109, 110, and 111, respectively; and
(b) a VL comprising CDR1, CDR2, and CDR3 comprising the amino acid sequences of SEQ ID NOs: 112, 113, and 114, respectively.

In another aspect, the present invention provides an antigen-binding site that binds FLT3, comprising:
(a) a VH comprising CDR1, CDR2, and CDR3 comprising the amino acid sequences of SEQ ID NOs: 117, 118, and 119, respectively; and
(b) a VL comprising CDR1, CDR2, and CDR3 comprising the amino acid sequences of SEQ ID NOs: 120, 121, and 122, respectively.

In another aspect, the present invention provides an antigen-binding site that binds FLT3, comprising:
(a) a VH comprising CDR1, CDR2, and CDR3 comprising the amino acid sequences of SEQ ID NOs: 87, 98, and 89, respectively; and
(b) a VL comprising CDR1, CDR2, and CDR3 comprising the amino acid sequences of SEQ ID NOs: 106, 92, and 93, respectively.

In another aspect, the present invention provides an antigen-binding site that binds FLT3, comprising:
(a) a VH comprising CDR1, CDR2, and CDR3 comprising the amino acid sequences of SEQ ID NOs: 62, 33, and 127, respectively; and
(b) a VL comprising CDR1, CDR2, and CDR3 comprising the amino acid sequences of SEQ ID NOs: 128, 129, and 130, respectively.

In another aspect, the present invention provides an antigen-binding site that binds FLT3, comprising:
(a) a VH comprising CDR1, CDR2, and CDR3 comprising the amino acid sequences of SEQ ID NOs: 132, 133, and 134, respectively; and
(b) a VL comprising CDR1, CDR2, and CDR3 comprising the amino acid sequences of SEQ ID NOs: 65, 66, and 46, respectively.

In another aspect, the present invention provides an antigen-binding site that competes with an antigen-binding site disclosed above.

In certain embodiments of the foregoing aspects, the antigen-binding site binds human FLT3 with a dissociation constant (K_{D}) smaller than or equal to 20 nM as measured by surface plasmon resonance (SPR). In certain embodiments, the antigen-binding site binds human FLT3 with a K_{D} smaller than or equal to 10 nM as measured by SPR. In certain embodiments, the antigen-binding site binds a human FLT3 variant comprising the amino acid sequence of SEQ ID NO:25. In certain embodiments, the antigen-binding site binds a human FLT3 variant comprising the amino acid sequence of SEQ ID NO:18. In certain embodiments, the antigen-binding site binds cynomolgus FLT3. In certain embodiments, the antigen-binding site does not compete with FLT3L for binding FLT3.

In certain embodiments, the antigen-binding site is present as a single-chain fragment variable (scFv). In certain embodiments, the scFv comprises an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, and 82.

In another aspect, the present invention provides a protein comprising an antigen-binding site disclosed herein. In certain embodiments, the protein further comprises an antibody heavy chain constant region. In certain embodiments, the antibody heavy chain constant region is a human IgG heavy chain constant region. In certain embodiments, the antibody heavy chain constant region is a human IgG1 heavy chain constant region. In certain embodiments, each polypeptide chain of the antibody heavy chain constant region comprises an amino acid sequence at least 90% identical to SEQ ID NO:21.

In certain embodiments, at least one polypeptide chain of the antibody heavy chain constant region comprises one or more mutations, relative to SEQ ID NO:21, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system. In certain embodiments, at least one polypeptide chain of the antibody heavy chain constant region comprises one or more mutations, relative to SEQ ID NO:21, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E, numbered according to the EU numbering system. In certain embodiments, one polypeptide chain of the antibody heavy chain constant region comprises one or more mutations, relative to SEQ ID NO:21, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to SEQ ID NO:21, at one or more positions selected from Q347, Y349, L351, S354, E356, E357, S364, T366, L368, K370, N390, K392, T394, D399, D401, F405, Y407, K409, T411, and K439, numbered according to the EU numbering system. In certain embodiments, one polypeptide chain of the antibody heavy chain constant region comprises K360E and K409W substitutions relative to SEQ ID NO:21; and the other polypeptide chain of the antibody heavy chain constant region comprises Q347R, D399V and F405T substitutions relative to SEQ ID NO:21, numbered according to the EU numbering system. In certain embodiments, one polypeptide chain of the antibody heavy chain constant region comprises a Y349C substitution relative to SEQ ID NO:21; and the other polypeptide chain of the antibody heavy chain constant region comprises an S354C substitution relative to SEQ ID NO:21, numbered according to the EU numbering system.

In another aspect, the present invention provides an antibody-drug conjugate comprising a protein disclosed herein and a drug moiety. In certain embodiments, the drug moiety is selected from the group consisting of auristatin, N-acetyl-γ calicheamicin, maytansinoid, pyrrolobenzodiazepine, and SN-38.

In another aspect, the present invention provides an immunocytokine comprising an antigen-binding site disclosed herein and a cytokine. In certain embodiments, the cytokine is selected from the group consisting of IL-2, IL-4, IL-10, IL-12, IL-15, TNF, and IFNα.

In another aspect, the present invention provides a bispecific T-cell engager comprising an antigen-binding site disclosed herein and an antigen-binding site that binds CD3.

In another aspect, the present invention provides a chimeric antigen receptor (CAR) comprising:
(a) the antigen-binding site disclosed herein;
(b) a transmembrane domain; and
(c) an intracellular signaling domain.

In certain embodiments, the transmembrane domain is selected from the transmembrane regions of the alpha, beta or zeta chain of the T-cell receptor, CD28, CD3 epsilon, CD45, CD4, CD5, CD8, CD9, CD16, CD22, FLT3, CD37, CD64, CD80, CD86, CD134, CD137, CD152, and CD154. In certain embodiments, the intracellular signaling domain comprises a primary signaling domain comprising a functional signaling domain of CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12. In certain embodiments, the intracellular signaling domain further comprises a costimulatory signaling domain comprising a functional signaling domain of a costimulatory receptor. In certain embodiments, the costimulatory receptor is selected from the group consisting of OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

In another aspect, the present invention provides an isolated nucleic acid encoding a CAR disclosed herein.

In another aspect, the present invention provides an expression vector comprising an isolated nucleic acid disclosed herein.

In another aspect, the present invention provides an immune effector cell comprising a nucleic acid or expression vector disclosed herein.

In another aspect, the present invention provides an immune effector cell expressing a CAR disclosed herein. In certain embodiments, the immune effector cell is a T cell. In certain embodiments, the T cell is a CD8⁺ T cell, a CD4⁺ T cell, or an NKT cell. In certain embodiments, the immune effector cell is an NK cell.

In another aspect, the present invention provides a pharmaceutical composition comprising a protein, antibody-drug conjugate, immunocytokine, bispecific T-cell engager, or immune effector cell disclosed herein; and a pharmaceutically acceptable carrier.

In another aspect, the present invention provides a method of treating cancer, the method comprising administering to a subject in need thereof an effective amount of a protein, antibody-drug conjugate, immunocytokine, bispecific T-cell engager, immune effector cell, or pharmaceutical composition disclosed herein.

In certain embodiments, the cancer is a hematologic malignancy. In certain embodiments, the hematologic malignancy is leukemia. In certain embodiments, the cancer is selected from the group consisting of acute myeloid leukemia (AML), acute lymphoblastic leukemia (ALL), myelodysplasia, acute T-lymphoblastic leukemia, and acute promyelocytic leukemia. In certain embodiments, the cancer expresses FLT3.

These and other aspects and advantages of the invention are illustrated by the following figures, detailed description and claims.

### DESCRIPTION OF THE DRAWINGS

The invention can be more completely understood with reference to the following drawings.

FIG. 1 is a set of sensograms showing SPR profiles of antibodies collected from the murine hybridomas supernatants binding to hFLT3.

FIG. 2 is a set of sensograms showing SPR profiles of antibodies collected from the murine mAb subclones binding to hFLT3.

FIG. 3 is a bar graph depicting the reduction of the ability of the candidate antibodies to bind FLT3-expressing EOL-1 cancer cells by saturating concentrations of soluble FLT3-ligand.

FIGS. 4A-4C are line graphs showing binding of anti-FLT3 antibody 1158 to FLT3-expressing cell lines RMA-hFLT3 (FIG. 4A), RMA-cFLT3 (FIG. 4B), and REH (FIG. 4C).

FIG. 5 is a line graphs showing binding of anti-FLT3 antibody 1158 to MOLM-13 cells, which expressed FLT3 with T227M mutation.

FIGS. 6A-6D are bar graphs showing NK cell-mediated lysis of FLT3-expressing cancer cell lines EOL-1 (FIG. 6A), REH (FIG. 6B), RS4-11 (FIG. 6C), and MV4-11 (FIG. 6D) in the presence of TriNKET F3′-1158 and its parental monoclonal antibody.

FIGS. 7A-7B are bar graphs showing FLT3 phosphorylation by TriNKET F3′-1158 and its parental monoclonal antibody in the absence (FIG. 7A) or presence (FIG. 7B) of FLT3-ligand. The FLT3-ligand sample in FIG. 7A serves as a positive control.

### DETAILED DESCRIPTION

The present invention provides antigen-binding sites that bind human FLT3 and optionally bind cynomolgus FLT3. These antigen-binding sites bind various epitopes in an extracellular domain of FLT3, and a few of these antigen-binding sites do not compete with FLT3-ligand (FLT3L) for such binding. Proteins and protein conjugates containing such antigen-binding sites, for example, antibodies, antibody-drug conjugates, bispecific T-cell engagers (BiTEs), and immunocytokines, as well as immune effector cells (e.g., T cells) expressing a protein containing such an antigen-binding site (e.g., a chimeric antigen receptor (CAR)), are useful for treating FLT3-associated diseases such as cancer.

The present invention provides antigen-binding proteins that bind FLT3 on a cancer cell and pharmaceutical compositions comprising such proteins, and therapeutic methods using such proteins and pharmaceutical compositions, including for the treatment of cancer. Various aspects of the present invention are set forth in the sections below; however, aspects of the invention described in one particular section are not to be limited to any particular section.

To facilitate an understanding of the present invention, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in antigen binding. In human antibodies, the antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide. All the amino acid positions in heavy or light chain variable regions disclosed herein are numbered according to Kabat numbering.

The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the present invention and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecyl sulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, “FLT3” (also known as FLK2, STK1, or CD135) refers to the protein of Uniprot Accession No. P36888 and related isoforms.

As used herein, “FLT3L” (also known as FLT3-ligand) refers to the protein of Uniprot Accession No. P49771 and related isoforms.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

Various features and aspects of the invention are discussed in more detail below.

### I. Antigen-Binding Site

In one aspect, the present invention provides an antigen-binding site that binds human FLT3. The VH, VL, CDR, and scFv sequences of exemplary antigen-binding sites are listed in Table 1. The CDR sequences are identified according to the Chothia numbering scheme.

TABLE 1
Sequences of Exemplary Antigen-Binding Sites that Bind FLT3

| Clone | VH | VL |
|---|---|---|
| 12H10.G7 | EVQLQESGPELVKPGASVKMS | NIVLTQSPASLAVSLGQRATISCR |
| | CKASGYTFTRYVMHWVKQRP | ASESVDTYGSSFVHWYQQKPGQP |
| | GQGLEWIGFINPYNDDTKYNE | PKLLIYLASNLESGVPARFSGSGS |
| | KFKGKATLTSDKSSSTAYMELS | RSDFTLTIDPVEADDAATYYCQQ |
| | SLTSEDSAVYHCARWRQLGSL | NNEEPWTFGGGTKLEIK |
| | DSWGQGTTLTVSS | [SEQ ID NO: 2] |
| | [SEQ ID NO: 1] | CDR1: RASESVDTYGSSFVH [SEQ ID NO: 6] |
| | CDR1: GYTFTRY [SEQ ID NO: 11] | CDR2: LASNLES [SEQ ID NO: 7] |
| | CDR2: NPYNDD [SEQ ID NO: 4] | CDR3: QQNNEEPWT [SEQ ID NO: 8] |
| | CDR3: WRQLGSLDS [SEQ ID NO: 5] | |
| Humanized | QVQLVQSGAEVKKPGASVKVS | DIVMTQSPASLAVSLGERATINCR |
| 12H10.G7 | CKASGYTFTRYVMHWVRQAP | ASESVDTYGSSFVHWYQQKPGQP |
| GB87/GB95 | GQRLEWMGFINPYNDDTKYNE | PKLLIYLASNLESGVPDRFSGSGS |
| (back | KFKGRVTITSDTSASTAYMELS | RTDFTLTISSLQAEDAATYYCQQN |
| mutations in | SLRSEDTAVYHCARWRQLGSL | NEEPWTFGGGTKVEIK [SEQ ID NO: 10] |
| VH and VL | DSWGQGTTVTVSS [SEQ ID NO: 9] | CDR1: RASESVDTYGSSFVH [SEQ ID NO: 6] |
| underlined) | CDR1: GYTFTRY [SEQ ID NO: 11] | CDR2: LASNLES[SEQ ID NO: 7] |
| | CDR2: NPYNDD [SEQ ID NO: 4] | CDR3: QQNNEEPWT [SEQ ID NO: 8] |
| | CDR3: WRQLGSLDS[SEQ ID NO: 5] | |
| scFv of | GB87 (VH-VL): | |
| humanized | QVQLVQSGAEVKKPGASVKVSCKASGYTFTRYVMHWVRQAPGQCL | |
| 12H10.G7 | EWMGFINPYNDDTKYNEKFKGRVTITSDTSASTAYMELSSLRSEDTA | |
| GB87/GB95 | VYHCARWRQLGSLDSWGQGTTVTVSSGGGGSGGGGSGGGGSGGG | |
| | GSDIVMTQSPASLAVSLGERATINCRASESVDTYGSSFVHWYQQKPG | |
| | QPPKLLIYLASNLESGVPDRFSGSGSRTDFTLTISSLQAEDAATYYCQ | |
| | QNNEEPWTFGCGTKVEIK | |
| | [SEQ ID NO: 3] | |
| | GB95 (VL-VH): | |
| | DIVMTQSPASLAVSLGERATINCRASESVDTYGSSFVHWYQQKPGQP | |
| | PKLLIYLASNLESGVPDRFSGSGSRTDFTLTISSLQAEDAATYYCQQN | |
| | NEEPWTFGCGTKVEIKGGGGSGGGGSGGGGSGGGGSQVQLVQSGA | |
| | EVKKPGASVKVSCKASGYTFTRYVMHWVRQAPGQCLEWMGFINPY | |
| | NDDTKYNEKFKGRVTITSDTSASTAYMELSSLRSEDTAVYHCARWR | |
| | QLGSLDSWGQGTTVTVSS | |
| | [SEQ ID NO: 12] | |
| Humanized | QVQLVQSGAEVKKPGASVKVS | DIVMTQSPASLAVSLGERATINCR |
| 12H10.G7 | CKASGYTFTRYVMHWVRQAP | ASESVDTYGSSFVHWYQQKPGQP |
| GB88/GB96 | GQRLEWMGFINPYNDDTKYNE | PKLLIYLASNLESGVPDRFSGSGS |
| (back | KFKGRVTITRDTSASTAYMELS | RTDFTLTISSLQAEDAATYYCQQN |
| mutations in | SLRSEDTAVYHCARWRQLGSL | NEEPWTFGGGTKVEIK |
| VH and VL | DSWGQGTTVTVSS | [SEQ ID NO: 10] |
| underlined) | [SEQ ID NO: 13] | CDR1: RASESVDTYGSSFVH [SEQ ID NO: 6] |
| | CDR1: GYTFTRY [SEQ ID NO: 11] | CDR2: LASNLES [SEQ ID NO: 7] |
| | CDR2: NPYNDD [SEQ ID NO: 4] | CDR3: QQNNEEPWT [SEQ ID NO: 8] |
| | CDR3: WRQLGSLDS [SEQ ID NO: 5] | |
| scFv of | GB88 (VH-VL): | |
| humanized | QVQLVQSGAEVKKPGASVKVSCKASGYTFTRYVMHWVRQAPGQCL | |
| 12H10.G7 | EWMGFINPYNDDTKYNEKFKGRVTITRDTSASTAYMELSSLRSEDTA | |
| GB88/GB96 | VYHCARWRQLGSLDSWGQGTTVTVSSGGGGSGGGGSGGGGSGGG | |
| | GSDIVMTQSPASLAVSLGERATINCRASESVDTYGSSFVHWYQQKPG | |
| | QPPKLLIYLASNLESGVPDRFSGSGSRTDFTLTISSLQAEDAATYYCQ | |
| | QNNEEPWTFGCGTKVEIK | |
| | [SEQ ID NO: 15] | |
| | GB96 (VL-VH): | |
| | DIVMTQSPASLAVSLGERATINCRASESVDTYGSSFVHWYQQKPGQP | |
| | PKLLIYLASNLESGVPDRFSGSGSRTDFTLTISSLQAEDAATYYCQQN | |
| | NEEPWTFGCGTKVEIKGGGGSGGGGSGGGGSGGGGSQVQLVQSGA | |
| | EVKKPGASVKVSCKASGYTFTRYVMHWVRQAPGQCLEWMGFINPY | |
| | NDDTKYNEKFKGRVTITRDTSASTAYMELSSLRSEDTAVYHCARWR | |
| | QLGSLDSWGQGTTVTVSS | |
| | [SEQ ID NO: 16} | |
| Humanized | QVQLVQSGAEVKKPGASVKVS | DIVMTQSPASLAVSLGERATINCR |
| 12H10.G7 | CKASGYTFTRYVMHWVRQAP | ASESVDTYGSSFVHWYQQKPGQP |
| GB89/GB97 | GQRLEWMGFINPYNDDTKYNE | PKLLIYLASNLESGVPDRFSGSGS |
| (back | KFKGRVTITSDTSASTAYMELS | RTDFTLTISSLQAEDAATYYCQQN |
| mutations in | SLRSEDTAVYYCARWRQLGSL | NEEPWTFGGGTKVEIK |
| VH and VL | DSWGQGTTVTVSS [SEQ ID NO: 17] | [SEQ ID NO: 10] |
| underlined) | CDR1: GYTFTRY [SEQ ID NO: 11] | CDR1: RASESVDTYGSSFVH [SEQ ID NO: 6] |
| | CDR2: NPYNDD [SEQ ID NO: 4] | CDR2: LASNLES [SEQ ID NO: 7] |
| | CDR3: WRQLGSLDS [SEQ ID NO: 5] | CDR3: QQNNEEPWT [SEQ ID NO: 8] |
| scFv of | GB89 (VH-VL): | |
| humanized | QVQLVQSGAEVKKPGASVKVSCKASGYTFTRYVMHWVRQAPGQCL | |
| 12H10.G7 | EWMGFINPYNDDTKYNEKFKGRVTITSDTSASTAYMELSSLRSEDTA | |
| GB89/GB97 | VYYCARWRQLGSLDSWGQGTTVTVSSGGGGSGGGGSGGGGSGGG | |
| | GSDIVMTQSPASLAVSLGERATINCRASESVDTYGSSFVHWYQQKPG | |
| | QPPKLLIYLASNLESGVPDRFSGSGSRTDFTLTISSLQAEDAATYYCQ | |
| | QNNEEPWTFGCGTKVEIK | |
| | [SEQ ID NO: 19] | |
| | GB97 (VL-VH): | |
| | DIVMTQSPASLAVSLGERATINCRASESVDTYGSSFVHWYQQKPGQP | |
| | PKLLIYLASNLESGVPDRFSGSGSRTDFTLTISSLQAEDAATYYCQQN | |
| | NEEPWTFGCGTKVEIKGGGGSGGGGSGGGGSGGGGSQVQLVQSGA | |
| | EVKKPGASVKVSCKASGYTFTRYVMHWVRQAPGQCLEWMGFINPY | |
| | NDDTKYNEKFKGRVTITSDTSASTAYMELSSLRSEDTAVYYCARWR | |
| | QLGSLDSWGQGTTVTVSS | |
| | [SEQ ID NO: 20] | |
| Humanized | QVQLVQSGAEVKKPGASVKVS | DIVMTQSPDSLAVSLGERATINCR |
| 12H10.G7 | CKASGYTFTRYVMHWVRQAP | ASESVDTYGSSFVHWYQQKPGQP |
| GB90/GB98 | GQRLEWMGFINPYNDDTKYNE | PKLLIYLASNLESGVPDRFSGSGS |
| (back | KFKGRVTITSDTSASTAYMELS | RTDFTLTISSLQAEDAATYYCQQN |
| mutations in | SLRSEDTAVYHCARWRQLGSL | NEEPWTFGGGTKVEIK |
| VH and VL | DSWGQGTTVTVSS | [SEQ ID NO: 22] |
| underlined) | [SEQ ID NO: 9] | CDR1: RASESVDTYGSSFVH [SEQ ID NO: 6] |
| | CDR1: GYTFTRY [SEQ ID NO: 11] | CDR2: LASNLES [SEQ ID NO: 7] |
| | CDR2: NPYNDD [SEQ ID NO: 4] | CDR3: QQNNEEPWT [SEQ ID NO: 8] |
| | CDR3: WRQLGSLDS [SEQ ID NO: 5] | |
| scFv of | GB90 (VH-VL): | |
| humanized | QVQLVQSGAEVKKPGASVKVSCKASGYTFTRYVMHWVRQAPGQCL | |
| 12H10.G7 | EWMGFINPYNDDTKYNEKFKGRVTITSDTSASTAYMELSSLRSEDTA | |
| GB90/GB98 | VYHCARWRQLGSLDSWGQGTTVTVSSGGGGSGGGGSGGGGSGGG | |
| | GSDIVMTQSPDSLAVSLGERATINCRASESVDTYGSSFVHWYQQKPG | |
| | QPPKLLIYLASNLESGVPDRFSGSGSRTDFTLTISSLQAEDAATYYCQ | |
| | QNNEEPWTFGCGTKVEIK [SEQ ID NO: 23] | |
| | GB98 (VL-VH): | |
| | DIVMTQSPDSLAVSLGERATINCRASESVDTYGSSFVHWYQQKPGQP | |
| | PKLLIYLASNLESGVPDRFSGSGSRTDFTLTISSLQAEDAATYYCQQN | |
| | NEEPWTFGCGTKVEIKGGGGSGGGGSGGGGSGGGGSQVQLVQSGA | |
| | EVKKPGASVKVSCKASGYTFTRYVMHWVRQAPGQCLEWMGFINPY | |
| | NDDTKYNEKFKGRVTITSDTSASTAYMELSSLRSEDTAVYHCARWR | |
| | QLGSLDSWGQGTTVTVSS | |
| | [SEQ ID NO: 24] | |
| Humanized | QVQLVQSGAEVKKPGASVKVS | DIVMTQSPASLAVSLGERATINCR |
| 12H10.G7 | CKASGYTFTRYVMHWVRQAP | ASESVDTYGSSFVHWYQQKPGQP |
| GB91/GB99 | GQRLEWMGFINPYNDDTKYNE | PKLLIYLASNLESGVPDRFSGSGS |
| (back | KFKGRVTITSDTSASTAYMELS | GTDFTLTISSLQAEDAATYYCQQN |
| mutations in | SLRSEDTAVYHCARWRQLGSL | NEEPWTFGGGTKVEIK |
| VH and VL | DSWGQGTTVTVSS | [SEQ ID NO: 26] |
| underlined) | [SEQ ID NO: 9] | CDR1: RASESVDTYGSSFVH [SEQ ID NO: 6] |
| | CDR1: GYTFTRY [SEQ ID NO: 11] | CDR2: LASNLES [SEQ ID NO: 7] |
| | CDR2: NPYNDD [SEQ ID NO: 4] | CDR3: QQNNEEPWT [SEQ ID NO: 8] |
| | CDR3: WRQLGSLDS [SEQ ID NO: 5] | |
| scFv of | GB91 (VH-VL): | |
| humanized | QVQLVQSGAEVKKPGASVKVSCKASGYTFTRYVMHWVRQAPGQCL | |
| 12H10.G7 | EWMGFINPYNDDTKYNEKFKGRVTITSDTSASTAYMELSSLRSEDTA | |
| GB91/GB99 | VYHCARWRQLGSLDSWGQGTTVTVSSGGGGSGGGGSGGGGSGGG | |
| | GSDIVMTQSPASLAVSLGERATINCRASESVDTYGSSFVHWYQQKPG | |
| | QPPKLLIYLASNLESGVPDRFSGSGSGTDFTLTISSLQAEDAATYYCQ | |
| | QNNEEPWTFGCGTKVEIK | |
| | [SEQ ID NO: 27] | |
| | GB99 (VL-VH): | |
| | DIVMTQSPASLAVSLGERATINCRASESVDTYGSSFVHWYQQKPGQP | |
| | PKLLIYLASNLESGVPDRFSGSGSGTDFTLTISSLQAEDAATYYCQQN | |
| | NEEPWTFGCGTKVEIKGGGGSGGGGSGGGGSGGGGSQVQLVQSGA | |
| | EVKKPGASVKVSCKASGYTFTRYVMHWVRQAPGQCLEWMGFINPY | |
| | NDDTKYNEKFKGRVTITSDTSASTAYMELSSLRSEDTAVYHCARWR | |
| | QLGSLDSWGQGTTVTVSS | |
| | [SEQ ID NO: 28] | |
| Humanized | QVQLVQSGAEVKKPGASVKVS | DIVMTQSPASLAVSLGERATINCR |
| 12H10.G7 | CKASGYTFTRYVMHWVRQAP | ASESVDTYGSSFVHWYQQKPGQP |
| GB92/GB100 | GQRLEWMGFINPYNDDTKYNE | PKLLIYLASNLESGVPDRFSGSGS |
| (back | KFKGRVTITSDTSASTAYMELS | RTDFTLTISSLQAEDVATYYCQQN |
| mutations in | SLRSEDTAVYHCARWRQLGSL | NEEPWTFGGGTKVEIK |
| VH and VL | DSWGQGTTVTVSS | [SEQ ID NO: 30] |
| underlined) | [SEQ ID NO: 9] | CDR1: RASESVDTYGSSFVH [SEQ ID NO: 6] |
| | CDR1: GYTFTRY [SEQ ID NO: 11] | CDR2: LASNLES [SEQ ID NO: 7] |
| | CDR2: NPYNDD [SEQ ID NO: 4] | CDR3: QQNNEEPWT [SEQ ID NO: 8] |
| | CDR3: WRQLGSLDS [SEQ ID NO: 5] | |
| scFv of | GB92 (VH-VL): | |
| humanized | QVQLVQSGAEVKKPGASVKVSCKASGYTFTRYVMHWVRQAPGQCL | |
| 12H10.G7 | EWMGFINPYNDDTKYNEKFKGRVTITSDTSASTAYMELSSLRSEDTA | |
| GB92/GB100 | VYHCARWRQLGSLDSWGQGTTVTVSSGGGGSGGGGSGGGGSGGG | |
| | GSDIVMTQSPASLAVSLGERATINCRASESVDTYGSSFVHWYQQKPG | |
| | QPPKLLIYLASNLESGVPDRFSGSGSRTDFTLTISSLQAEDVATYYCQ | |
| | QNNEEPWTFGCGTKVEIK | |
| | [SEQ ID NO: 31] | |
| | GB100 (VL-VH): | |
| | DIVMTQSPASLAVSLGERATINCRASESVDTYGSSFVHWYQQKPGQP | |
| | PKLLIYLASNLESGVPDRFSGSGSRTDFTLTISSLQAEDVATYYCQQN | |
| | NEEPWTFGCGTKVEIKGGGGSGGGGSGGGGSGGGGSQVQLVQSGA | |
| | EVKKPGASVKVSCKASGYTFTRYVMHWVRQAPGQCLEWMGFINPY | |
| | NDDTKYNEKFKGRVTITSDTSASTAYMELSSLRSEDTAVYHCARWR | |
| | QLGSLDSWGQGTTVTV | |
| | [SEQ ID NO: 32] | |
| Humanized | QVQLVQSGAEVKKPGASVKVS | DIVMTQSPASLAVSLGERATINCR |
| 12H10.G7 | CKASGYTFTRYVMHWVRQAP | ASESVDTYGSSFVHWYQQKPGQP |
| GB93/GB101 | GQRLEWMGFINPYNDDTKYNE | PKLLIYLASNLESGVPDRFSGSGS |
| (back | KFKGRVTITSDTSASTAYMELS | RTDFTLTISSLQAEDAAVYYCQQ |
| mutations in | SLRSEDTAVYHCARWRQLGSL | NNEEPWTFGGGTKVEIK |
| VH and VL | DSWGQGTTVTVSS | [SEQ ID NO: 34] |
| underlined) | [SEQ ID NO: 9] | CDR1: RASESVDTYGSSFVH [SEQ ID NO: 6] |
| | CDR1: GYTFTRY [SEQ ID NO: 11] | CDR2: LASNLES [SEQ ID NO: 7] |
| | CDR2: NPYNDD [SEQ ID NO: 4] | CDR3: QQNNEEPWT [SEQ ID NO: 8] |
| | CDR3: WRQLGSLDS [SEQ ID NO: 5] | |
| scFv of | GB93 (VH-VL): | |
| humanized | QVQLVQSGAEVKKPGASVKVSCKASGYTFTRYVMHWVRQAPGQCL | |
| 12H10.G7 | EWMGFINPYNDDTKYNEKFKGRVTITSDTSASTAYMELSSLRSEDTA | |
| GB93/GB101 | VYHCARWRQLGSLDSWGQGTTVTVSSGGGGSGGGGSGGGGSGGG | |
| | GSDIVMTQSPASLAVSLGERATINCRASESVDTYGSSFVHWYQQKPG | |
| | QPPKLLIYLASNLESGVPDRFSGSGSRTDFTLTISSLQAEDAAVYYCQ | |
| | QNNEEPWTFGCGTKVEIK | |
| | [SEQ ID NO: 35] | |
| | GB101 (VL-VH): | |
| | DIVMTQSPASLAVSLGERATINCRASESVDTYGSSFVHWYQQKPGQP | |
| | PKLLIYLASNLESGVPDRFSGSGSRTDFTLTISSLQAEDAAVYYCQQN | |
| | NEEPWTFGCGTKVEIKGGGGSGGGGSGGGGSGGGGSQVQLVQSGA | |
| | EVKKPGASVKVSCKASGYTFTRYVMHWVRQAPGQCLEWMGFINPY | |
| | NDDTKYNEKFKGRVTITSDTSASTAYMELSSLRSEDTAVYHCARWR | |
| | QLGSLDSWGQGTTVTVSS | |
| | [SEQ ID NO: 36] | |
| Humanized | QVQLVQSGAEVKKPGASVKVS | DIVMTQSPDSLAVSLGERATINCR |
| 12H10.G7 | CKASGYTFTRYVMHWVRQAP | ASESVDTYGSSFVHWYQQKPGQP |
| GB94/GB102 | GQRLEWMGFINPYNDDTKYNE | PKLLIYLASNLESGVPDRFSGSGS |
| | KFKGRVTITRDTSASTAYMELS | GTDFTLTISSLQAEDVAVYYCQQ |
| | SLRSEDTAVYYCARWRQLGSL | NNEEPWTFGGGTK VEIK |
| | DSWGQGTTVTVSS | [SEQ ID NO: 38] |
| | [SEQ ID NO: 37] | CDR1: RASESVDTYGSSFVH [SEQ ID NO: 6] |
| | CDR1: GYTFTRY [SEQ ID NO: 11] | CDR2: LASNLES [SEQ ID NO: 7] |
| | CDR2: NPYNDD [SEQ ID NO: 4] | CDR3: QQNNEEPWT [SEQ ID NO: 8] |
| | CDR3: WRQLGSLDS [SEQ ID NO: 5] | |
| scFv of*** | GB94 (VH-VL): | |
| humanized | QVQLVQSGAEVKKPGASVKVSCKASGYTFTRYVMHWVRQAPGQCL | |
| 12H10.G7 | EWMGFINPYNDDTKYNEKFKGRVTITRDTSASTAYMELSSLRSEDTA | |
| GB94/GB102 | VYYCARWRQLGSLDSWGQGTTVTVSSGGGGSGGGGSGGGGSGGG | |
| | GSDIVMTQSPDSLAVSLGERATINCRASESVDTYGSSFVHWYQQKPG | |
| | QPPKLLIYLASNLESGVPDRFSGSGSGTDFTLTISSLQAEDVAVYYCQ | |
| | QNNEEPWTFGCGTKVEIK | |
| | [SEQ ID NO: 39] | |
| | GB102 (VL-VH): | |
| | DIVMTQSPDSLAVSLGERATINCRASESVDTYGSSFVHWYQQKPGQP | |
| | PKLLIYLASNLESGVPDRFSGSGSGTDFTLTISSLQAEDVAVYYCQQN | |
| | NEEPWTFGCGTKVEIKGGGGSGGGGSGGGGSGGGGSQVQLVQSGA | |
| | EVKKPGASVKVSCKASGYTFTRYVMHWVRQAPGQCLEWMGFINPY | |
| | NDDTKYNEKFKGRVTITRDTSASTAYMELSSLRSEDTAVYYCARWR | |
| | QLGSLDSWGQGTTVTVSS | |
| | [SEQ ID NO: 40] | |
| Humanized | QVQLVQSGAEVKKPGASVKVS | DIVMTQSPDSLAVSLGERATINCR |
| 12H10.G7 | CKASGYTFTRYVMHWVRQAP | ASESVDTYGSSFVHWYQQKPGQP |
| GB102 | GQRLEWMGFINPYNDDTKYNE | PKLLIYLASNLESGVPDRFSGSGS |
| D101E | KFKGRVTITRDTSASTAYMELS | GTDFTLTISSLQAEDVAVYYCQQ |
| | SLRSEDTAVYYCARWRQLGSL | NNEEPWTFGCGTKVEIK |
| | ESWGQGTTVTVSS | [SEQ ID NO: 42] |
| | [SEQ ID NO: 41] | CDR1: RASESVDTYGSSFVH [SEQ ID NO: 6] |
| | CDR1: GYTFTRY [SEQ ID NO: 11] | CDR2: LASNLES [SEQ ID NO: 7] |
| | CDR2: NPYNDD [SEQ ID NO: 4] | CDR3: QQNNEEPWT [SEQ ID NO: 8] |
| | CDR3: WRQLGSLDS [SEQ ID NO: 5] | |
| scFv of | VH-VL: | |
| humanized | QVQLVQSGAEVKKPGASVKVSCKASGYTFTRYVMHWVRQAPGQCL | |
| 12H10.G7 | EWMGFINPYNDDTKYNEKFKGRVTITRDTSASTAYMELSSLRSEDTA | |
| GB102 | VYYCARWRQLGSLESWGQGTTVTVSSGGGGSGGGGSGGGGSGGGG | |
| D101E | SDIVMTQSPDSLAVSLGERATINCRASESVDTYGSSFVHWYQQKPGQ | |
| | PPKLLIYLASNLESGVPDRFSGSGSGTDFTLTISSLQAEDVAVYYCQQ | |
| | NNEEPWTFGCGTKVEIK | |
| | [SEQ ID NO: 43] | |
| | VL-VH: | |
| | DIVMTQSPDSLAVSLGERATINCRASESVDTYGSSFVHWYQQKPGQP | |
| | PKLLIYLASNLESGVPDRFSGSGSGTDFTLTISSLQAEDVAVYYCQQN | |
| | NEEPWTFGCGTKVEIKGGGGSGGGGSGGGGSGGGGSQVQLVQSGA | |
| | EVKKPGASVKVSCKASGYTFTRYVMHWVRQAPGQCLEWMGFINPY | |
| | NDDTKYNEKFKGRVTITRDTSASTAYMELSSLRSEDTAVYYCARWR | |
| | QLGSLESWGQGTTVTVSS | |
| | [SEQ ID NO: 44] | |
| Humanized | QVQLVQSGAEVKKPGASVKVS | DIVMTQSPDSLAVSLGERATINCR |
| 12H10.G7 | CKASGYTFTRYVIHWVRQAPG | ASESVDTYGSSFVHWYQQKPGQP |
| GB102 M34I | QRLEWMGFINPYNDDTKYNEK | PKLLIYLASNLESGVPDRFSGSGS |
| | FKGRVTITRDTSASTAYMELSS | GTDFTLTISSLQAEDVAVYYCQQ |
| | LRSEDTAVYYCARWRQLGSLD | NNEEPWTFGCGTKVEIK |
| | SWGQGTTVTVSS | [SEQ ID NO: 42] |
| | [SEQ ID NO: 45] | CDR1: RASESVDTYGSSFVH [SEQ ID NO: 6] |
| | CDR1: GYTFTRY [SEQ ID NO: 11] | CDR2: LASNLES [SEQ ID NO: 7] |
| | CDR2: NPYNDD [SEQ ID NO: 4] | CDR3: QQNNEEPWT [SEQ ID NO: 8] |
| | CDR3: WRQLGSLDS [SEQ ID NO: 5] | |
| scFv of | VH-VL: | |
| humanized | QVQLVQSGAEVKKPGASVKVSCKASGYTFTRYVIHWVRQAPGQCLE | |
| 12H10.G7 | WMGFINPYNDDTKYNEKFKGRVTITRDTSASTAYMELSSLRSEDTAV | |
| GB102 M34I | YYCARWRQLGSLDSWGQGTTVTVSSGGGGSGGGGSGGGGSGGGGS | |
| | DIVMTQSPDSLAVSLGERATINCRASESVDTYGSSFVHWYQQKPGQP | |
| | PKLLIYLASNLESGVPDRFSGSGSGTDFTLTISSLQAEDVAVYYCQQN | |
| | NEEPWTFGCGTKVEIK | |
| | [SEQ ID NO: 47] | |
| | VL-VH: | |
| | DIVMTQSPDSLAVSLGERATINCRASESVDTYGSSFVHWYQQKPGQP | |
| | PKLLIYLASNLESGVPDRFSGSGSGTDFTLTISSLQAEDVAVYYCQQN | |
| | NEEPWTFGCGTKVEIKGGGGSGGGGSGGGGSGGGGSQVQLVQSGA | |
| | EVKKPGASVKVSCKASGYTFTRYVIHWVRQAPGQCLEWMGFINPYN | |
| | DDTKYNEKFKGRVTITRDTSASTAYMELSSLRSEDTAVYYCARWRQ | |
| | LGSLDSWGQGTTVTVSS | |
| | [SEQ ID NO: 48] | |
| Humanized | QVQLVQSGAEVKKPGASVKVS | DIVMTQSPDSLAVSLGERATINCR |
| 12H10.G7 | CKASGYTFTRYVIHWVRQAPG | ASESVDTYGSSFVHWYQQKPGQP |
| GB102 | QRLEWMGFINPYNDDTKYNEK | PKLLIYLASNLESGVPDRFSGSGS |
| M34I/D101E | FKGRVTITRDTSASTAYMELSS | GTDFTLTISSLQAEDVAVYYCQQ |
| | LRSEDTAVYYCARWRQLGSLE | NNEEPWTFGCGTKVEIK |
| | SWGQGTTVTVSS | [SEQ ID NO: 42] |
| | [SEQ ID NO: 49] | CDR1: RASESVDTYGSSFVH [SEQ ID NO: 6] |
| | CDR1: GYTFTRY [SEQ ID NO: 11] | CDR2: LASNLES [SEQ ID NO: 7] |
| | CDR2: NPYNDD [SEQ ID NO: 4] | CDR3: QQNNEEPWT [SEQ ID NO: 8] |
| | CDR3: WRQLGSLES [SEQ ID NO: 50] | |
| scFv of | VH-VL: | |
| humanized | QVQLVQSGAEVKKPGASVKVSCKASGYTFTRYVIHWVRQAPGQCLE | |
| 12H10.G7 | WMGFINPYNDDTKYNEKFKGRVTITRDTSASTAYMELSSLRSEDTAV | |
| GB102 | YYCARWRQLGSLESWGQGTTVTVSSGGGGSGGGGSGGGGSGGGGS | |
| M34I/D101E | DIVMTQSPDSLAVSLGERATINCRASESVDTYGSSFVHWYQQKPGQP | |
| | PKLLIYLASNLESGVPDRFSGSGSGTDFTLTISSLQAEDVAVYYCQQN | |
| | NEEPWTFGCGTKVEIK | |
| | [SEQ ID NO: 51] | |
| | VL-VH: | |
| | DIVMTQSPDSLAVSLGERATINCRASESVDTYGSSFVHWYQQKPGQP | |
| | PKLLIYLASNLESGVPDRFSGSGSGTDFTLTISSLQAEDVAVYYCQQN | |
| | NEEPWTFGCGTKVEIKGGGGSGGGGSGGGGSGGGGSQVQLVQSGA | |
| | EVKKPGASVKVSCKASGYTFTRYVIHWVRQAPGQCLEWMGFINPYN | |
| | DDTKYNEKFKGRVTITRDTSASTAYMELSSLRSEDTAVYYCARWRQ | |
| | LGSLESWGQGTTVTVSS | |
| | [SEQ ID NO: 52] | |
| Humanized | QVQLVQSGAEVKKPGASVKVS | DIVMTQSPDSLAVSLGERATINCR |
| 12H10.G7 | CKASGYTFTRYVX₁HWVRQAP | ASESVDTYGSSFVHWYQQKPGQP |
| consensus 1 | GQRLEWMGFINPYNDDTKYNE | PKLLIYLASNLESGVPDRFSGSGS |
| | KFKGRVTITRDTSASTAYMELS | GTDFTLTISSLQAEDVAVYYCQQ |
| | SLRSEDTAVYYCARWRQLGSL | NNEEPWTFGCGTKVEIK |
| | X₂SWGQGTTVTVSS | [SEQ ID NO: 42] |
| | where X₁ is M or I, and X₂ is E | CDR1: RASESVDTYGSSFVH [SEQ ID NO: 6] |
| | or D | CDR2: LASNLES [SEQ ID NO: 7] |
| | [SEQ ID NO: 53] | CDR3: QQNNEEPWT [SEQ ID NO: 8] |
| | CDR1: GYTFTRY [SEQ ID NO: 11] | |
| | CDR2: NPYNDD [SEQ ID NO: 4] | |
| | CDR3: WRQLGSLXS, where X is | |
| | E or D [SEQ ID NO: 55] | |
| Humanized | QVQLVQSGAEVKKPGASVKVS | DIVMTQSPX₁SLAVSLGERATINC |
| 12H10.G7 | CKASGYTFTRYVMHWVRQAP | RASESVDTYGSSFVHWYQQKPGQ |
| consensus 2 | GQRLEWMGFINPYNDDTKYNE | PPKLLIYLASNLESGVPDRFSGSGS |
| | KFKGRVTITX₁DTSASTAYMEL | X₂TDFTLTISSLQAEDX₃AX₄YYCQ |
| | SSLRSEDTAVYX₂CARWRQLGS | QNNEEPWTFGGGTKVEIK |
| | LDSWGQGTTVTVSS | where X1 is A or D, X₂ is R or G, and |
| | where X₁ is S or R, and X₂ is Y | X₃ is A or V, and X₄ is T or V |
| | or H | [SEQ ID NO: 57] |
| | [SEQ ID NO: 56] | CDR1: RASESVDTYGSSFVH [SEQ ID NO: 6] |
| | CDR1: GYTFTRY [SEQ ID NO: 11] | CDR2: LASNLES [SEQ ID NO: 7] |
| | CDR2: NPYNDD [SEQ ID NO: 4] | CDR3: QQNNEEPWT [SEQ ID NO: 8] |
| | CDR3: WRQLGSLDS [SEQ ID NO: 51 | |
| Humanized | QVQLVQSGAEVKKPGASVKVS | DIVMTQSPDSLAVSLGERATINCR |
| 12H10.G7 | CKASGYTFTRYVX₁HWVRQAP | ASESVDTYGSSFVHWYQQKPGQP |
| consensus 3 | GQCLEWMGFINPYNDDTKYNE | PKLLIYLASNLESGVPDRFSGSGS |
| | KFKGRVTITRDTSASTAYMELS | GTDFTLTISSLQAEDVAVYYCQQ |
| | SLRSEDTAVYYCARWRQLGSL | NNEEPWTFGCGTKVEIK |
| | X₂SWGQGTTVTVSS | [SEQ ID NO: 42] |
| | where X₁ is M or I, and X2 is E | CDR1: RASESVDTYGSSFVH [SEQ ID NO: 6] |
| | or D | CDR2: LASNLES [SEQ ID NO: 7] |
| | [SEQ ID NO: 58] | CDR3: QQNNEEPWT [SEQ ID NO: 8] |
| | CDR1: GYTFTRY [SEQ ID NO: 11] | |
| | CDR2: NPYNDD [SEQ ID NO: 4] | |
| | CDR3: WRQLGSLXS, where X is | |
| | E or D [SEQ ID NO: 55] | |
| 14A5.E8 | EVQLQESGAELVQPGASVRLSC | QIVLTQSPAIMSASPGEKVTMTCS |
| | KASGYTFTSYWINWVKQRPGQ | ASSSVSYMHWYQQKSGTSPKRWI |
| | GLEWIGNIYPGSSIINYNENFKN | YDTSKLASGVPARFSGSGSGTSYS |
| | RATLTVDTSSSTAYMQLSSLTS | LTISSMEAEDAATYYCQQWTSKS |
| | DDSAVYYCARRVVYLYFDYW | PTFGGGTKLEIK |
| | GQGTTLTVSS [SEQ ID NO: 60] | [SEQ ID NO: 61] |
| | CDR1: GYTFTSY [SEQ ID NO: 62] | CDR1: SASSSVSYMH [SEQ ID NO: 65] |
| | CDR2: YPGSSI [SEQ ID NO: 63] | CDR2: DTSKLAS [SEQ ID NO: 66] |
| | CDR3: RVVYLYFDY [SEQ ID NO: 64] | CDR3: QQWTSKSPT [SEQ ID NO: 67] |
| Humanized | QVQLVQSGAEVKKPGASVKVS | EIVLTQSPATLSLSPGEKATLSCSA |
| 14A5.E8 | CKVSGYTFTSYWINWVRQRPG | SSSVSYMHWYQQKPGQAPRLLIY |
| 1551/1552 | KGLEWMGNIYPGSSIINYNENF | DTSKLASGIPARFSGSGSGTSFTLT |
| (back | KNRVTMTVDTSSDTAYMELSS | ISSLEPEDAAVYYCQQWTSKSPTF |
| mutations in | LRSEDTAVYYCARRVVYLYFD | GGGTKVEIK [SEQ ID NO: 69] |
| VH and VL | YWGQGTLVTVSS | CDR1: SASSSVSYMH [SEQ ID NO: 65] |
| underlined) | [SEQ ID NO: 68] | CDR2: DTSKLAS[SEQ ID NO: 66] |
| | CDR1: GYTFTSY [SEQ ID NO: 62] | CDR3: QQWTSKSPT [SEQ ID NO: 67] |
| | CDR2: YPGSSI [SEQ ID NO: 63] | |
| | CDR3: RVVYLYFDY [SEQ ID NO: 64] | |
| scFv of | 1551 (VH-VL): | |
| humanized | QVQLVQSGAEVKKPGASVKVSCKVSGYTFTSYWINWVRQRPGKCL | |
| 14A5.E8 | EWMGNIYPGSSIINYNENFKNRVTMTVDTSSDTAYMELSSLRSEDTA | |
| 1551/1552 | VYYCARRVVYLYFDYWGQGTLVTVSSGGGGSGGGGSGGGGSGGG | |
| | GSEIVLTQSPATLSLSPGEKATLSCSASSSVSYMHWYQQKPGQAPRL | |
| | LIYDTSKLASGIPARFSGSGSGTSFTLTISSLEPEDAAVYYCQQWTSKS | |
| | PTFGCGTKVEIK | |
| | [SEQ ID NO: 70] | |
| | 1552 (VL-VH): | |
| | EIVLTQSPATLSLSPGEKATLSCSASSSVSYMHWYQQKPGQAPRLLIY | |
| | DTSKLASGIPARFSGSGSGTSFTLTISSLEPEDAAVYYCQQWTSKSPTF | |
| | GCGTKVEIKGGGGSGGGGSGGGGSGGGGSQVQLVQSGAEVKKPGA | |
| | SVKVSCKVSGYTFTSYWINWVRQRPGKCLEWMGNIYPGSSIINYNEN | |
| | FKNRVTMTVDTSSDTAYMELSSLRSEDTAVYYCARRVVYLYFDYW | |
| | GQGTLVTVSS | |
| | [SEQ ID NO: 71] | |
| Humanized | QVQLVQSGAEVKKPGASVKVS | EIVLTQSPATLSLSPGERATLSCSA |
| 14A5.E8 | CKVSGYTFTSYWINWVRQAPG | SSSVSYMHWYQQKPGQAPRLLIY |
| 1553/1554 | KGLEWMGNIYPGSSIINYNENF | DTSKLASGIPARFSGSGSGTDFTLT |
| | KNRVTMTEDTSTDTAYMELSS | ISSLEPEDFAVYYCQQWTSKSPTF |
| | LRSEDTAVYYCARRVVYLYFD | GGGTKVEIK [SEQ ID NO: 73] |
| | YWGQGTLVTVSS | CDR1: SASSSVSYMH [SEQ ID NO: 65] |
| | [SEQ ID NO: 72] | CDR2: DTSKLAS [SEQ ID NO: 66] |
| | CDR1: GYTFTSY [SEQ ID NO: 62] | CDR3: QQWTSKSPT [SEQ ID NO: 67] |
| | CDR2: YPGSSI [SEQ ID NO: 63] | |
| | CDR3: RVVYLYFDY [SEQ ID NO: 64] | |
| scFv of | 1553 (VH-VL): | |
| humanized | QVQLVQSGAEVKKPGASVKVSCKVSGYTFTSYWINWVRQAPGKCL | |
| 14A5.E8 | EWMGNIYPGSSIINYNENFKNRVTMTEDTSTDTAYMELSSLRSEDTA | |
| 1553/1554 | VYYCARRVVYLYFDYWGQGTLVTVSSGGGGSGGGGSGGGGSGGG | |
| | GSEIVLTQSPATLSLSPGERATLSCSASSSVSYMHWYQQKPGQAPRLL | |
| | IYDTSKLASGIPARFSGSGSGTDFTLTISSLEPEDFAVYYCQQWTSKSP | |
| | TFGCGTKVEIK | |
| | [SEQ ID NO: 74] | |
| | 1554 (VL-VH): | |
| | EIVLTQSPATLSLSPGERATLSCSASSSVSYMHWYQQKPGQAPRLLIY | |
| | DTSKLASGIPARFSGSGSGTDFTLTISSLEPEDFAVYYCQQWTSKSPTF | |
| | GCGTKVEIKGGGGSGGGGSGGGGSGGGGSQVQLVQSGAEVKKPGA | |
| | SVKVSCKVSGYTFTSYWINWVRQAPGKCLEWMGNIYPGSSIINYNEN | |
| | FKNRVTMTEDTSTDTAYMELSSLRSEDTAVYYCARRVVYLYFDYW | |
| | GQGTLVTVSS | |
| | [SEQ ID NO: 75] | |
| Humanized | QVQLVQSGAEVKKPGASVKVS | EIVLTQSPATLSLSPGERATLSCSA |
| 14A5.E8 | CKVSGYTFPYYWINWVRQAPG | SSSVSYIHWYQQKPGQAPRLLIYD |
| 1689 (affinity | KGLEWMGNIYPGSSIINYNENF | TSKLASGIPARFSGSGSGTDFTLTI |
| matured from | KNRVTMTEDTSTDTAYMELSS | SSLEPEDFAVYYCQQWTSKSPTFG |
| 1553) | LRSEDTAVYYCARRNVYLTFD | GGTKVEIK |
| | YWGQGTLVTVSS | [SEQ ID NO: 77] |
| | [SEQ ID NO: 76] | CDR1: SASSSVSYIH [SEQ ID NO: 80] |
| | CDR1: GYTFPYY [SEQ ID NO: 78] | CDR2: DTSKLAS [SEQ ID NO: 66] |
| | CDR2: YPGSSI [SEQ ID NO: 63] | CDR3: QQWTSKSPT [SEQ ID NO: 67] |
| | CDR3: RNVYLTFDY [SEQ ID NO: 79] | |
| scFv of | VH-VL: | |
| humanized | QVQLVQSGAEVKKPGASVKVSCKVSGYTFPYYWINWVRQAPGKCL | |
| 14A5.E8 | EWMGNIYPGSSIINYNENFKNRVTMTEDTSTDTAYMELSSLRSEDTA | |
| 1689 (affinity | VYYCARRNVYLTFDYWGQGTLVTVSSGGGGSGGGGSGGGGSGGGGS | |
| matured from | EIVLTQSPATLSLSPGERATLSCSASSSVSYIHWYQQKPGQAPRLLIYD | |
| 1553) | TSKLASGIPARFSGSGSGTDFTLTISSLEPEDFAVYYCQQWTSKSPTFG | |
| | CGTKVEIK | |
| | [SEQ ID NO: 81] | |
| | VL-VH: | |
| | EIVLTQSPATLSLSPGERATLSCSASSSVSYIHWYQQKPGQAPRLLIYD | |
| | TSKLASGIPARFSGSGSGTDFTLTISSLEPEDFAVYYCQQWTSKSPTFG | |
| | CGTKVEIKGGGGSGGGGSGGGGSGGGGSQVQLVQSGAEVKKPGAS | |
| | VKVSCKVSGYTFPYYWINWVRQAPGKCLEWMGNIYPGSSIINYNEN | |
| | FKNRVTMTEDTSTDTAYMELSSLRSEDTAVYYCARRNVYLTFDYW | |
| | GQGTLVTVSS | |
| | [SEQ ID NO: 82] | |
| Humanized | QVQLVQSGAEVKKPGASVKVS | EIVLTQSPATLSLSPGERATLSCSA |
| 14A5.E8 | CKVSGYTFX₁X₂YWINWVRQX₃ | SSSVSYXHWYQQKPGQAPRLLIY |
| consensus | PGKX₄LEWMGNIYPGSSIINYNE | DTSKLASGIPARFSGSGSGTDFTLT |
| | NFKNRVTMTX₅DTSX₆DTAYM | ISSLEPEDFAVYYCQQWTSKSPTF |
| | ELSSLRSEDTAVYYCARRX₇VY | GGGTKVEIK |
| | LX₈FDYWGQGTLVTVSS | where X is M or I |
| | where X₁ is P or T, X₂ is S or Y, X₃ | [SEQ ID NO: 84] |
| | is A or R, X₄ is C or G, X₅ is V or | CDR1: SASSSVSYXH, wherein X is |
| | E, X₆ is S or T, X₇ is N or V, and | M or I [SEQ ID NO: 86] |
| | X₈ is T or Y | CDR2: DTSKLAS [SEQ ID NO: 66] |
| | [SEQ ID NO: 29] | CDR3: QQWTSKSPT [SEQ ID NO: 67] |
| | CDR1: GYTFX₁X₂Y, where X₁ is | |
| | P or T, and X₂ is S or Y | |
| | [SEQ ID NO: 59] | |
| | CDR2: YPGSSI [SEQ ID NO: 63] | |
| | CDR3: RX₁VYLX₂FDY, where X₁ | |
| | is N or V, and X₂ is T or Y | |
| | [SEQ ID NO: 54] | |
| 11F4.B9 | EVQLQESGPELVKPGASVKISC | DIVLTQSPASLAVSLGQRATISCR |
| | KASGYSFTGYYIHWVKQGPEK | ASESVDIYGNSFMHWYQQKPGQP |
| | SLEWIGEIIPSTGSTIYNQKFKA | PKLLIYRASNLESGIPARFSGSGSR |
| | KATLTVDKSSSTAYLQLKSLTS | TDFTLTINPVEADDVATYYCQQS |
| | EDSAVYYCERWGDYYGRDYW | NEDPRTFGGGTKLEIK |
| | GQGTSVTVSS | [SEQ ID NO: 90] |
| | [SEQ ID NO: 85] | CDR1: RASESVDIYGNSFMH |
| | CDR1: GYSFTGY [SEQ ID NO: 87] | [SEQ ID NO: 91] |
| | CDR2: IPSTGS [SEQ ID NO: 88] | CDR2: RASNLES [SEQ ID NO: 92] |
| | CDR3: WGDYYGRDY [SEQ ID NO: 89] | CDR3: QQSNEDPRT [SEQ ID NO: 93] |
| Humanized | QVQLVQSGAEVKKPGASVKVS | DIVMTQSPASLAVSLGERATINCR |
| 11F4.B9 | CKASGYSFTGYYIHWVRQGPG | ASESVDIYGNSFMHWYQQKPGQP |
| (back | QGLEWMGEIIPSTGSTIYAQKF | PKLLIYRASNLESGVPDRFSGSGS |
| mutations | QGRVTMTRDTSTSTVYMELSS | RTDFTLTINSLQAEDVATYYCQQS |
| underlined) | LRSEDTAVYYCERWGDYYGR | NEDPRTFGGGTKVEIK |
| | DYWGQGTLVTVSS | [SEQ ID NO: 94] |
| | [SEQ ID NO: 14] | CDR1: RASESVDIYGNSFMH |
| | CDR1: GYSFTGY [SEQ ID NO: 87] | [SEQ ID NO: 91] |
| | CDR2: IPSTGS [SEQ ID NO: 88] | CDR2: RASNLES [SEQ ID NO: 92] |
| | CDR3: WGDYYGRDY [SEQ ID NO: 89] | CDR3: QQSNEDPRT [SEQ ID NO: 93] |
| 4A4.A3 | QVTLKESGPGILQPSQTLSLTCS | RIVMTQSPTTMAASPGEKITITCSA |
| | FSGFSLTTYGMGVGWIRQPSG | SSSISSIYLHWYQQKPGFSPKLLIF |
| | KGLEWLANIWFNDNKYYNSTL | RTSDLASGVPPRFGGSGSGTSYSL |
| | KSRLTISKDTSNNQVFLKISSVD | TIGTMEAEDVATYYCQQGSSFPR |
| | TTDTATYYCAQITTVVGTFDY | TFGGGTKLEIK |
| | WGQGSPLTVSP [SEQ ID NO: 95] | [SEQ ID NO: 96] |
| | CDR1: GFSLTTYGM [SEQ ID NO: 97] | CDR1: SASSSISSIYLH [SEQ ID NO: 101] |
| | CDR2: WFNDN [SEQ ID NO: 99] | CDR2: RTSDLAS [SEQ ID NO: 102] |
| | CDR3: ITTVVGTEDY [SEQ ID NO: 100] | CDR3: QQGSSFPRT [SEQ ID NO: 103] |
| 4A4.H7 | EVQLQESGPELVKPGASVKISC | DIVLTQSPASLAVSLGQRATISCR |
| | KASGYSFTGYYIHWVKQSPEES | ASETVDTHGNSFMHWYQQKPGQ |
| | LEWIGEIYPNTGITTYNQKFTA | PPKLLIYRASNLESGIPARFSGSGS |
| | KATLTVDKSSNTAYMQLKSLT | RTDFTLTINPVEADDVATYYCQQ |
| | SEDSAVYYCTRWGDYYGRDY | SNEDPRTFGGGTKLEIK |
| | WGQGTSVTVSS | [SEQ ID NO: 105] |
| | [SEQ ID NO: 104] | CDR1: RASETVDTHGNSFMH |
| | CDR1: GYSFTGY [SEQ ID NO: 87] | [SEQ ID NO: 106] |
| | CDR2: YPNTGI [SEQ ID NO: 98] | CDR2: RASNLES [SEQ ID NO: 92] |
| | CDR3: WGDYYGRDY [SEQ ID NO: 89] | CDR3: QQSNEDPRT [SEQ ID NO: 93] |
| 15A11.C8 | EVQLQESGGGLVKTGGSRKLS | DIQMTQTTSSLSASLGDRVTIRCR |
| | CAASGFTFSDYGMHWVRHTPE | ASQDITNYLNWYQQKPDGAVKL |
| | KGLEWVVYISSGGNTIFYTDTV | LISYTSILQSGVPSRFSGSGSGTDY |
| | KGRFTISRDNAKNTLFLQMTSL | SLTISNLEQGDVATYFCQQGSSLP |
| | RSEDTAVYFCVRQGYYYAMD | WTFGGGTKLEIK |
| | YWGQGASVTVSS | [SEQ ID NO: 108] |
| | [SEQ ID NO: 107] | CDR1: RASQDITNYLN [SEQ ID NO: 112] |
| | CDR1: GFTFSDY [SEQ ID NO: 109] | CDR2: YTSILQS [SEQ ID NO: 113] |
| | CDR2: SSGGNT [SEQ ID NO: 110] | CDR3: QQGSSLPWT [SEQ ID NO: 114] |
| | CDR3: QGYYYAMDY [SEQ ID NO: 111] | |
| 12C9.E5 | EVQLQESGAELVRPGASVKLSC | DVVMTQTPLSLSVTIGQPASISCK |
| | KASGYIFTDYEIHWVKQTPVH | SSQSLLYSDGETYLNWLQQRPGQ |
| | GLEWIGAIDPETGITAYSQKFK | SPKRLMYQVSKLDPGIPDRFSGSG |
| | GKATLTTDTSSSTAYMEFRSLT | SETDFTLKISR VEAEDLGIYYCLQ |
| | SEDSAVYYCTRGGLLYWGQGT | GTFYPHTFGGGTKLEIK |
| | SVTVSS | [SEQ ID NO: 116] |
| | [SEQ ID NO: 115] | CDR1: KSSQSLLYSDGETYLN |
| | CDR1: GYIFTDY [SEQ ID NO: 117] | [SEQ ID NO: 120] |
| | CDR2: DPETGI [SEQ ID NO: 118] | CDR2: QVSKLDP [SEQ ID NO: 121] |
| | CDR3: GGLLY [SEQ ID NO: 119] | CDR3: LQGTFYPHT [SEQ ID NO: 122] |
| 1A2.A3 | EVQLQESGPELVKPGASVKISC | DIVLTQSPASLAVSLGQRATISCR |
| | KASGYSFTGYYIHWVKQSPEES | ASETVDTHGNSFMHWYQQKPGQ |
| | LEWIGEIYPNTGITTYNQKFTA | PPKLLIYRASNLESGIPARFSGSGS |
| | KATLTVDKSSNTAYMQLKSLT | RTDFTLTINPVEADDVATYYCQQ |
| | SEDSAVYYCTRWGDYYGRDY | SNEDPRTFGGGTKLEIK |
| | WGQGTSVTVSS | [SEQ ID NO: 124] |
| | [SEQ ID NO: 123] | CDR1: RASETVDTHGNSFMH |
| | CDR1: GYSFTGY [SEQ ID NO: 87] | [SEQ ID NO: 106] |
| | CDR2: YPNTGI [SEQ ID NO: 98] | CDR2: RASNLES [SEQ ID NO: 92] |
| | CDR3: WGDYYGRDY [SEQ ID NO: 89] | CDR3: QQSNEDPRT [SEQ ID NO: 93] |
| 4H2.E3 | EVQLQESGPELVKPGASVKMS | GIVMTQTTPSVPVTPGESVSISCRS |
| | CKASGYTFTSYLMHWMKQKP | SKSLLHSNGNTYLYWFLQRPGQS |
| | GQGLEWIGYINPYSDGIKYNEK | PQLLIYRMSNLASGVPDRFSGSGS |
| | FRDKATLTSDKSSNTAYMELSS | GTTFTLRISRVEAEDVGVYYCMQ |
| | LTSEDSAVYYCAHSSGYVGYA | HLEYPFTFGSGTKLEIK |
| | MDYWGQGTSVTVSS | [SEQ ID NO: 126] |
| | [SEQ ID NO: 125] | CDR1: RSSKSLLHSNGNTYLY |
| | CDR1: GYTFTSY [SEQ ID NO: 62] | [SEQ ID NO: 128] |
| | CDR2: NPYSDG [SEQ ID NO: 33] | CDR2: RMSNLAS [SEQ ID NO: 129] |
| | CDR3: SSGYVGYAMDY | CDR3: MQHLEYPFT [SEQ ID NO: 130] |
| | [SEQ ID NO: 127] | |
| 14H8.E7 | EVQLQESGAELVKPGASVKLS | QIVLTQSPAIMSASPGEKVTMTCS |
| | CKASGYTFTNYWINWLKQRPG | ASSSVSYMHWYQQKSGTSPKRWI |
| | QGLEWIGNIYPGSTIINYNEKFK | FDTSKLASGVPVRFSGSGSGTSYS |
| | NKATLTVDTSSSTAYMQLSSLT | LTITNMETEDAATYYCQQWSSKS |
| | SDDSAVYYCARRVVYLYFDSW | PTFGGGTKLEIK [SEQ ID NO: 83] |
| | GQGTTLTVSS | CDR1: SASSSVSYMH [SEQ ID NO: 65] |
| | [SEQ ID NO: 131] | CDR2: DTSKLAS [SEQ ID NO: 66] |
| | CDR1: GYTFTNY [SEQ ID NO: 132] | CDR3: QQWSSKSPT [SEQ ID NO: 46] |
| | CDR2: YPGSTI [SEQ ID NO: 133] | |
| | CDR3: RVVYLYFDS [SEQ ID NO: 134] | |

In certain embodiments, the antigen-binding site of the present invention comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 1, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VH of the same antibody disclosed in Table 1. In certain embodiments, the antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J Mol Biol 196: 901-917), MacCallum (see MacCallum R M et al., (1996) J Mol Biol 262: 732-745), or any other CDR determination method known in the art, of the VH and VL sequences of an antibody discloses in Table 1. In certain embodiments, the antigen-binding site comprises the heavy chain CDR1, CDR2, and CDR3 and the light chain CDR1, CDR2, and CDR3 of an antibody disclosed in Table 1.

In certain embodiments, the antigen-binding site of the present invention is related to 12H10.G7. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:1, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:2. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively.

In certain embodiments, the antigen-binding site of the present invention is related to GB87 or GB95. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:10. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 3 or 12.

In certain embodiments, the antigen-binding site of the present invention is related to GB88 or GB96. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:13, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:10. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 15 or 16.

In certain embodiments, the antigen-binding site of the present invention is related to GB89 or GB97. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:17, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:10. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 19 or 20.

In certain embodiments, the antigen-binding site of the present invention is related to GB90 and GB98. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:22. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 23 or 24.

In certain embodiments, the antigen-binding site of the present invention is related to GB91 and GB99. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:26. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 27 or 28.

In certain embodiments, the antigen-binding site of the present invention is related to GB92 or GB100. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:30. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 31 or 32.

In certain embodiments, the antigen-binding site of the present invention is related to GB93 or GB101. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:34. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 35 or 36.

In certain embodiments, the antigen-binding site of the present invention is related to GB94 or GB102. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:37, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:38. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 39 or 40.

In certain embodiments, the antigen-binding site of the present invention is related to GB102 D101E. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:41, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:42. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 43 or 44.

In certain embodiments, the antigen-binding site of the present invention is related to GB102 M34I. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:45, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:42. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 47 or 48.

In certain embodiments, the antigen-binding site of the present invention is related to GB102 M34I/D101E. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:49, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:42. In certain embodiments, the VH comprises CDR1, CDR2, and CDR3 comprising the amino acid sequences of SEQ ID NOs: 11, 4, and 50, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 50, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 51 or 52.

In certain embodiments, the antigen-binding site of the present invention is related to humanized 12H10.G7. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:53, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:42. In certain embodiments, the VH comprises CDR1, CDR2, and CDR3 comprising the amino acid sequences of SEQ ID NOs: 11, 4, and 55, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 55, respectively; and (b) a VL that comprises CDR1, CDR2, and CDR3 comprising the amino acid sequences of SEQ ID NOs: 6, 7, and 8, respectively.

In certain embodiments, the antigen-binding site of the present invention is related to humanized 12H10.G7. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:56, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:57. In certain embodiments, the VH comprises CDR1, CDR2, and CDR3 comprising the amino acid sequences of SEQ ID NOs: 11, 4, and 5, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 5, respectively; and (b) a VL that comprises CDR1, CDR2, and CDR3 comprising the amino acid sequences of SEQ ID NOs: 6, 7, and 8, respectively.

In certain embodiments, the antigen-binding site of the present invention is related to humanized 12H10.G7. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:58, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:42. In certain embodiments, the VH comprises CDR1, CDR2, and CDR3 comprising the amino acid sequences of SEQ ID NOs: 11, 4, and 55, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 11, 4, and 55, respectively; and (b) a VL that comprises CDR1, CDR2, and CDR3 comprising the amino acid sequences of SEQ ID NOs: 6, 7, and 8, respectively.

In certain embodiments, the antigen-binding site of the present invention is related to 14A5.E8. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:60, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:61. In certain embodiments, the VH comprises CDR1, CDR2, and CDR3 comprising the amino acid sequences of SEQ ID NOs: 62, 63, and 64, respectively. In certain embodiments, the VL comprises CDR1, CDR2, and CDR3 comprising the amino acid sequences of SEQ ID NOs: 65, 66, and 67, respectively. In certain embodiments, the antigen-binding site comprises (a) a VH that comprises CDR1, CDR2, and CDR3 comprising the amino acid sequences of SEQ ID NOs: 62, 63, and 64, respectively; and (b) a VL that comprises CDR1, CDR2, and CDR3 comprising the amino acid sequences of SEQ ID NOs: 65, 66, and 67, respectively.

In certain embodiments, the antigen-binding site of the present invention is related to mAb 1551 or 1552. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:68, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:69. In certain embodiments, the VH comprises CDR1, CDR2, and CDR3 comprising the amino acid sequences of SEQ ID NOs: 62, 63, and 64, respectively. In certain embodiments, the VL comprises CDR1, CDR2, and CDR3 comprising the amino acid sequences of SEQ ID NOs: 65, 66, and 67, respectively. In certain embodiments, the antigen-binding site comprises (a) a VH that comprises CDR1, CDR2, and CDR3 comprising the amino acid sequences of SEQ ID NOs: 62, 63, and 64, respectively; and (b) a VL that comprises CDR1, CDR2, and CDR3 comprising the amino acid sequences of SEQ ID NOs: 65, 66, and 67, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 70 or 71.

In certain embodiments, the antigen-binding site of the present invention is related to mAb 1553 or 1554. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:72, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:73. In certain embodiments, the VH comprises CDR1, CDR2, and CDR3 comprising the amino acid sequences of SEQ ID NOs: 62, 63, and 64, respectively. In certain embodiments, the VL comprises CDR1, CDR2, and CDR3 comprising the amino acid sequences of SEQ ID NOs: 65, 66, and 67, respectively. In certain embodiments, the antigen-binding site comprises (a) a VH that comprises CDR1, CDR2, and CDR3 comprising the amino acid sequences of SEQ ID NOs: 62, 63, and 64, respectively; and (b) a VL that comprises CDR1, CDR2, and CDR3 comprising the amino acid sequences of SEQ ID NOs: 65, 66, and 67, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 74 or 75.

In certain embodiments, the antigen-binding site of the present invention is related to mAb 1689. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:76, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:77. In certain embodiments, the VH comprises CDR1, CDR2, and CDR3 comprising the amino acid sequences of SEQ ID NOs: 78, 63, and 79, respectively. In certain embodiments, the VL comprises CDR1, CDR2, and CDR3 comprising the amino acid sequences of SEQ ID NOs: 80, 66, and 67, respectively. In certain embodiments, the antigen-binding site comprises (a) a VH that comprises CDR1, CDR2, and CDR3 comprising the amino acid sequences of SEQ ID NOs: 78, 63, and 79, respectively; and (b) a VL that comprises CDR1, CDR2, and CDR3 comprising the amino acid sequences of SEQ ID NOs: 80, 66, and 67, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 81 or 82.

In certain embodiments, the antigen-binding site of the present invention is related to humanized 14A5.E8. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:29, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:84. In certain embodiments, the VH comprises CDR1, CDR2, and CDR3 comprising the amino acid sequences of SEQ ID NOs: 59, 63, and 54, respectively. In certain embodiments, the VL comprises CDR1, CDR2, and CDR3 comprising the amino acid sequences of SEQ ID NOs: 86, 66, and 67, respectively. In certain embodiments, the antigen-binding site comprises (a) a VH that comprises CDR1, CDR2, and CDR3 comprising the amino acid sequences of SEQ ID NOs: 59, 63, and 54, respectively; and (b) a VL that comprises CDR1, CDR2, and CDR3 comprising the amino acid sequences of SEQ ID NOs: 86, 66, and 67, respectively.

In certain embodiments, the antigen-binding site of the present invention is related to 11F4.B9. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:85, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:90. In certain embodiments, the VH comprises CDR1, CDR2, and CDR3 comprising the amino acid sequences of SEQ ID NOs: 87, 88, and 89, respectively. In certain embodiments, the VL comprises CDR1, CDR2, and CDR3 comprising the amino acid sequences of SEQ ID NOs: 91, 92, and 93, respectively. In certain embodiments, the antigen-binding site comprises (a) a VH that comprises CDR1, CDR2, and CDR3 comprising the amino acid sequences of SEQ ID NOs: 87, 88, and 89, respectively; and (b) a VL that comprises CDR1, CDR2, and CDR3 comprising the amino acid sequences of SEQ ID NOs: 91, 92, and 93, respectively.

In certain embodiments, the antigen-binding site of the present invention is related to humanized 11F4.B9. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:14, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:94. In certain embodiments, the VH comprises CDR1, CDR2, and CDR3 comprising the amino acid sequences of SEQ ID NOs: 87, 88, and 89, respectively. In certain embodiments, the VL comprises CDR1, CDR2, and CDR3 comprising the amino acid sequences of SEQ ID NOs: 91, 92, and 93, respectively. In certain embodiments, the antigen-binding site comprises (a) a VH that comprises CDR1, CDR2, and CDR3 comprising the amino acid sequences of SEQ ID NOs: 87, 88, and 89, respectively; and (b) a VL that comprises CDR1, CDR2, and CDR3 comprising the amino acid sequences of SEQ ID NOs: 91, 92, and 93, respectively.

In certain embodiments, the antigen-binding site of the present invention is related to 4A4.A3. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:95, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:96. In certain embodiments, the VH comprises CDR1, CDR2, and CDR3 comprising the amino acid sequences of SEQ ID NOs: 97, 99, and 100, respectively. In certain embodiments, the VL comprises CDR1, CDR2, and CDR3 comprising the amino acid sequences of SEQ ID NOs: 101, 102, and 103, respectively. In certain embodiments, the antigen-binding site comprises (a) a VH that comprises CDR1, CDR2, and CDR3 comprising the amino acid sequences of SEQ ID NOs: 97, 99, and 100, respectively; and (b) a VL that comprises CDR1, CDR2, and CDR3 comprising the amino acid sequences of SEQ ID NOs: 101, 102, and 103, respectively.

In certain embodiments, the antigen-binding site of the present invention is related to 4A4.H7. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:104, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:105. In certain embodiments, the VH comprises CDR1, CDR2, and CDR3 comprising the amino acid sequences of SEQ ID NOs: 87, 98, and 89, respectively. In certain embodiments, the VL comprises CDR1, CDR2, and CDR3 comprising the amino acid sequences of SEQ ID NOs: 106, 92, and 93, respectively. In certain embodiments, the antigen-binding site comprises (a) a VH that comprises CDR1, CDR2, and CDR3 comprising the amino acid sequences of SEQ ID NOs: 87, 98, and 89, respectively; and (b) a VL that comprises CDR1, CDR2, and CDR3 comprising the amino acid sequences of SEQ ID NOs: 106, 92, and 93, respectively.

In certain embodiments, the antigen-binding site of the present invention is related to 15A11.C8. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:107, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:108. In certain embodiments, the VH comprises CDR1, CDR2, and CDR3 comprising the amino acid sequences of SEQ ID NOs: 109, 110, and 111, respectively. In certain embodiments, the VL comprises CDR1, CDR2, and CDR3 comprising the amino acid sequences of SEQ ID NOs: 112, 113, and 114, respectively. In certain embodiments, the antigen-binding site comprises (a) a VH that comprises CDR1, CDR2, and CDR3 comprising the amino acid sequences of SEQ ID NOs: 109, 110, and 111, respectively; and (b) a VL that comprises CDR1, CDR2, and CDR3 comprising the amino acid sequences of SEQ ID NOs: 112, 113, and 114, respectively.

In certain embodiments, the antigen-binding site of the present invention is related to 12C9.E5. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:115, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:116. In certain embodiments, the VH comprises CDR1, CDR2, and CDR3 comprising the amino acid sequences of SEQ ID NOs: 117, 118, and 119, respectively. In certain embodiments, the VL comprises CDR1, CDR2, and CDR3 comprising the amino acid sequences of SEQ ID NOs: 120, 121, and 122, respectively. In certain embodiments, the antigen-binding site comprises (a) a VH that comprises CDR1, CDR2, and CDR3 comprising the amino acid sequences of SEQ ID NOs: 117, 118, and 119, respectively; and (b) a VL that comprises CDR1, CDR2, and CDR3 comprising the amino acid sequences of SEQ ID NOs: 120, 121, and 122, respectively.

In certain embodiments, the antigen-binding site of the present invention is related to 1A2.A3. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:123, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:124. In certain embodiments, the VH comprises CDR1, CDR2, and CDR3 comprising the amino acid sequences of SEQ ID NOs: 87, 98, 89, respectively. In certain embodiments, the VL comprises CDR1, CDR2, and CDR3 comprising the amino acid sequences of SEQ ID NOs: 106, 92, 93, respectively. In certain embodiments, the antigen-binding site comprises (a) a VH that comprises CDR1, CDR2, and CDR3 comprising the amino acid sequences of SEQ ID NOs: 87, 98, 89, respectively; and (b) a VL that comprises CDR1, CDR2, and CDR3 comprising the amino acid sequences of SEQ ID NOs: 106, 92, 93, respectively.

In certain embodiments, the antigen-binding site of the present invention is related to 4H2.E3. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:125, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:126. In certain embodiments, the VH comprises CDR1, CDR2, and CDR3 comprising the amino acid sequences of SEQ ID NOs: 62, 33, and 127, respectively. In certain embodiments, the VL comprises CDR1, CDR2, and CDR3 comprising the amino acid sequences of SEQ ID NOs: 128, 129, and 130, respectively. In certain embodiments, the antigen-binding site comprises (a) a VH that comprises CDR1, CDR2, and CDR3 comprising the amino acid sequences of SEQ ID NOs: 62, 33, and 127, respectively; and (b) a VL that comprises CDR1, CDR2, and CDR3 comprising the amino acid sequences of SEQ ID NOs: 128, 129, and 130, respectively.

In certain embodiments, the antigen-binding site of the present invention is related to 14H8.E7. For example, in certain embodiments, the antigen-binding site of the present invention comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:131, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:83. In certain embodiments, the VH comprises CDR1, CDR2, and CDR3 comprising the amino acid sequences of SEQ ID NOs: 132, 133, and 134, respectively. In certain embodiments, the VL comprises CDR1, CDR2, and CDR3 comprising the amino acid sequences of SEQ ID NOs: 65, 66, and 46, respectively. In certain embodiments, the antigen-binding site comprises (a) a VH that comprises CDR1, CDR2, and CDR3 comprising the amino acid sequences of SEQ ID NOs: 132, 133, and 134, respectively; and (b) a VL that comprises CDR1, CDR2, and CDR3 comprising the amino acid sequences of SEQ ID NOs: 65, 66, and 46, respectively.

In each of the foregoing embodiments, it is contemplated herein that the VH and/or VL sequences that together bind FLT3 may contain amino acid alterations (e.g., at least 1, 2, 3, 4, 5, or 10 amino acid substitutions, deletions, or additions) in the framework regions of the VH and/or VL without affecting their ability to bind to FLT3 significantly.

In certain embodiments, the antigen-binding site of percent invention binds FLT3 (e.g., human FLT3) with a K_{D} (i.e., dissociation constant) of 1 nM or lower, 5 nM or lower, or 10 nM or lower, 15 nM or lower, or 20 nM or lower, as measured by surface plasmon resonance (SPR) (e.g., using the method described in Example 1 infra) or by bio-layer interferometry (BLI), and/or binds FLT3 from a body fluid, tissue, and/or cell of a subject. In certain embodiments, any of the foregoing isolated antibodies has a K_{d} (i.e., off-rate, also called K_{off}) equal to or lower than 1×10⁻⁵, 1×10⁻⁴, 1×10⁻³, 5×10⁻³, 0.01, 0.02, or 0.05 1/s, as measured by SPR (e.g., using the method described in Example 1 infra) or by BLI.

In certain embodiments, an antigen-binding site of the present invention, e.g., an antigen-binding site related to 12H10.G7, GB87, GB88, GB89, GB90, GB91, GB92, GB93, GB94, GB95, GB96, GB97, GB98, GB99, GB100, GB101, GB102, GB102 M34I, GB102 D101E, GB102 M34I/D101E, or a humanized 12H10.G7 disclosed above, binds a human FLT3 variant having a T227M mutation or the extracellular region thereof. The amino acid sequence of the extracellular region of hFLT3-T227M is

(SEQ ID NO: 25)
NQDLPVIKCVLINHKNNDSSVGKSSSYPMVSESPEDLGCALRPQSSGTV
YEAAAVEVDVSASITLQVLVDAPGNISCLWVFKHSSLNCQPHFDLQNRG
VVSMVILKMTETQAGEYLLFIQSEATNYTILFTVSIRNTLLYTLRRPYF
RKMENQDALVCISESVPEPIVEWVLCDSQGESCKEESPAVVKKEEKVLH
ELFGMDIRCCARNELGRECTRLFTIDLNQTPQTTLPQLFLKVGEPLWIR
CKAVHVNHGFGLTWELENKALEEGNYFEMSTYSTNRTMIRILFAFVSSV
ARNDTGYYTCSSSKHPSQSALVTIVEKGFINATNSSEDYEIDQYEEFCF
SVRFKAYPQIRCTWTFSRKSFPCEQKGLDNGYSISKFCNHKHQPGEYIF
HAENDDAQFTKMFTLNIRRKPQVLAEASASQASCFSDGYPLPSWTWKKC
SDKSPNCTEEITEGVWNRKANRKVFGQWVSSSTLNMSEAIKGFLVKCCA
YNSLGTSCETILLNSPGPFPFIQDNIS.

In certain embodiments, an antigen-binding site of the present invention, e.g., an antigen-binding site related to 12H10.G7, GB87, GB88, GB89, GB90, GB91, GB92, GB93, GB94, GB95, GB96, GB97, GB98, GB99, GB100, GB101, GB102, GB102 M34I, GB102 D101E, GB102 M34I/D101E, or a humanized 12H10.G7 disclosed above, binds a human FLT3 variant having an ITD mutation or the extracellular region thereof. The amino acid sequence of the extracellular region of hFLT3-ITD 15

(SEQ ID NO: 18)
NQDLPVIKCVLINHKNNDSSVGKSSSYPMVSESPEDLGCALRPQSSGTV
YEAAAVEVDVSASITLQVLVDAPGNISCLWVFKHSSLNCQPHFDLQNRG
VVSMVILKMTETQAGEYLLFIQSEATNYTILFTVSIRNTLLYTLRRPYF
RKMENQDALVCISESVPEPIVEWVLCDSQGESCKEESPAVVKKEEKVLH
ELFGTDIRCCARNELGRECTRLFTIDLNQTPQTTLPQLFLKVGEPLWIR
CKAVHVNHGFGLTWELENKALEEGNYFEMSTYSTNRTMIRILFAFVSSV
ARNDTGYYTCSSSKHPSQSALVTIVEKGFINATNSSEDYEIDQYEEFCF
SVRFKAYPQIRCTWTFSRKSFPCEQKGLDNGYSISKFCNHKHQPGEYIF
HAENDDAQFTKMFTLNIRRKPQVLAEASASQASCFSDGYPLPSWTWKKC
SDKSPNCTEEITEGVWNRKANRKVFGQWVSSSTLNMSEAIKGFLVKCCA
YNSLGTSCETILLNSPGPFPFIQDNIS.

In certain embodiments, an antigen-binding site of the present invention, e.g., an antigen-binding site related to 12H10.G7, GB87, GB88, GB89, GB90, GB91, GB92, GB93, GB94, GB95, GB96, GB97, GB98, GB99, GB100, GB101, GB102, GB102 M34I, GB102 D101E, GB102 M34I/D101E, a humanized 12H10.G7, 14A5.E8, 1551, 1552, 1553, 1554, 1689, a humanized 14A5.E8, 11F4.B9, 4A4.A3, 4A4.H7, 15A11.C8, 1A2.A3, 4H2.E3, or 14H8.E7 disclosed above, binds cynomolgus FLT3.

In certain embodiments, an antigen-binding site of the present invention, e.g., an antigen-binding site related to 12H10.G7, GB87, GB88, GB89, GB90, GB91, GB92, GB93, GB94, GB95, GB96, GB97, GB98, GB99, GB100, GB101, GB102, GB102 M34I, GB102 D101E, GB102 M34I/D101E, a humanized 12H10.G7, 14A5.E8, 1551, 1552, 1553, 1554, 1689, a humanized 14A5.E8, 11F4.B9, 4A4.A3, 4A4.H7, 12C9.E5, 1A2.A3, 4H2.E3, or 14H8.E7 disclosed above, does not compete with FLT3L for binding FLT3.

In another aspect, the present invention provides an antigen-binding site that competes for binding to FLT3 (e.g., human FLT3, cynomolgus FLT3) with an antigen-binding site described above. In certain embodiments, the antigen-binding site of the present invention competes with an antigen-binding site related to 1A2.A3 disclosed above for binding to FLT3. In one embodiment, the antigen-binding site competes with 1A2.A3 for binding to FLT3. In certain embodiments, the antigen-binding site of the present invention competes with an antigen-binding site related to 4A4.A3 disclosed above for binding to FLT3. In one embodiment, the antigen-binding site competes with 4A4.A3 for binding to FLT3. In certain embodiments, the antigen-binding site of the present invention competes with an antigen-binding site related to 4H2.E3 disclosed above for binding to FLT3. In one embodiment, the antigen-binding site competes with 4H2.E3 for binding to FLT3. In certain embodiments, the antigen-binding site of the present invention competes with an antigen-binding site related to 11F4.B9 disclosed above for binding to FLT3. In one embodiment, the antigen-binding site competes with 11F4.B9 for binding to FLT3.

Proteins with Antigen-Binding Sites

An antigen-binding site disclosed herein can be present in an antibody or antigen-binding fragment thereof. The antibody can be a monoclonal antibody, a chimeric antibody, a diabody, a Fab fragment, a Fab′ fragment, or F(ab′)₂ fragment, an Fv, a bispecific antibody, a bispecific Fab2, a bispecific (mab)2, a humanized antibody, an artificially-generated human antibody, bispecific T-cell engager, bispecific NK cell engager, a single chain antibody (e.g., single-chain Fv fragment or scFv), triomab, knobs-into-holes (kih) IgG with common light chain, crossmab, ortho-Fab IgG, DVD-Ig, 2 in 1-IgG, IgG-scFv, sdFv2-Fc, bi-nanobody, tandAb, dual-affinity retargeting antibody (DART), DART-Fc, scFv-HSA-scFv (where HSA=human serum albumin), or dock-and-lock (DNL)-Fab3.

In certain embodiments, an antigen-binding site disclosed herein is linked to an amino acid sequence at least 90% (e.g., 90%, 91%, 92%, 93%, 94%, 95%, 96%, 97%, 98%, 99%, or 100%) identical to an antibody constant region, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, an antigen-binding site disclosed herein can be linked to a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has effector function and can fix complement. In other embodiments the antibody does not recruit effector cells or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In certain embodiments, the antigen-binding site is linked to an IgG constant region including hinge, CH2 and CH3 domains with or without a CH1 domain. In some embodiments, the amino acid sequence of the constant region is at least 90% (e.g., 90%, 91%, 92%, 93%, 94%, 95%, 96%, 97%, 98%, 99%, or 100%) identical to a human antibody constant region, such as an human IgG1 constant region, a human IgG2 constant region, a human IgG3 constant region, or a human IgG4 constant region. In one embodiment, the antibody Fc domain or a portion thereof sufficient to bind CD16 comprises an amino acid sequence at least 90% (e.g., at least 91%, at least 92%, at least 93%, at least 94%, at least 95%, at least 96%, at least 97%, at least 98%, at least 99%, or 100%) identical to wild-type human IgG1 Fc sequence DKTHTCPPCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPAPIE KTISKAKGQPREPQVYTLPPSRDELTKNQVSLTCLVKGFYPSDIAVEWESNGQPENN YKTTPPVLDSDGSFFLYSKLTVDKSRWQQGNVFSCSVMHEALHNHYTQKSLSLSPG (SEQ ID NO:21). In some other embodiments, the amino acid sequence of the constant region is at least 90% (e.g., at least 91%, at least 92%, at least 93%, at least 94%, at least 95%, at least 96%, at least 97%, at least 98%, at least 99%, or 100%) identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

In certain embodiments, the antigen-binding site is linked to a portion of an antibody Fc domain sufficient to bind CD16. Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

In some embodiments, the antibody constant domain comprises a CH2 domain and a CH3 domain of an IgG antibody, for example, a human IgG1 antibody. In some embodiments, mutations are introduced in the antibody constant domain to enable heterodimerization with another antibody constant domain. For example, if the antibody constant domain is derived from the constant domain of a human IgG1, the antibody constant domain can comprise an amino acid sequence at least 90% (e.g., at least 91%, at least 92%, at least 93%, at least 94%, at least 95%, at least 96%, at least 97%, at least 98%, at least 99%, or 100%) identical to amino acids 234-332 of a human IgG1 antibody, and differs at one or more positions selected from the group consisting of Q347, Y349, L351, S354, E356, E357, K360, Q362, S364, T366, L368, K370, N390, K392, T394, D399, S400, D401, F405, Y407, K409, T411, and K439. All the amino acid positions in an Fc domain or hinge region disclosed herein are numbered according to EU numbering.

To facilitate formation of an asymmetric protein, Fc domain heterodimerization is contemplated. Mutations (e.g., amino acid substitutions) in the Fc domain that promote heterodimerization are described, for example, in International Application Publication No. WO2019157366, which is not incorporated herein by reference.

The proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the first immunoglobulin light chain can be cloned into a third expression vector; a fourth nucleic acid sequence encoding the second immunoglobulin light chain can be cloned into a fourth expression vector; the first, second, third and fourth expression vectors can be stably transfected together into host cells to produce the multimeric proteins.

To achieve the highest yield of the proteins, different ratios of the first, second, third and fourth expression vectors can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of a protein comprising an antigen-binding site disclosed herein. The protein can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

Accordingly, in another aspect, the present invention provides one or more isolated nucleic acids comprising sequences encoding an immunoglobulin heavy chain and/or immunoglobulin light chain variable region of any one of the foregoing antibodies. The invention provides one or more expression vectors that express the immunoglobulin heavy chain and/or immunoglobulin light chain variable region of any one of the foregoing antibodies. Similarly the invention provides host cells comprising one or more of the foregoing expression vectors and/or isolated nucleic acids.

In certain embodiments, the antibody binds FLT3 with a K_{D} of 20 nM, 15 nM, 10 nM, 9 nM, 8 nM, 7 nM, 6 nM, 5 nM, 4 nM, 3 nM, 2 nM, 1 nM or lower, as measured using standard binding assays, for example, surface plasmon resonance or bio-layer interferometry. In certain embodiments the antibody binds EBI3 from a body fluid, tissue and/or cell of a subject.

Competition assays for determining whether an antibody binds to the same epitope as, or competes for binding with a disclosed antibody are known in the art. Exemplary competition assays include immunoassays (e.g., ELISA assays, RIA assays), surface plasmon resonance (e.g., BIAcore analysis), bio-layer interferometry, and flow cytometry.

Typically, a competition assay involves the use of an antigen (e.g., a human FLT3 protein or fragment thereof) bound to a solid surface or expressed on a cell surface, a test FLT3-binding antibody and a reference antibody. The reference antibody is labeled and the test antibody is unlabeled. Competitive inhibition is measured by determining the amount of labeled reference antibody bound to the solid surface or cells in the presence of the test antibody. Usually the test antibody is present in excess (e.g., 1×, 5×, 10×, 20× or 100×). Antibodies identified by competition assay (e.g., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

A competition assay can be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In certain embodiments, isolated human antibodies contain one or more somatic mutations. In these cases, antibodies can be modified to a human germline sequence to optimize the antibody (e.g., by a process referred to as germlining).

Generally, an optimized antibody has at least the same, or substantially the same, affinity for the antigen as the non-optimized (or parental) antibody from which it was derived. Preferably, an optimized antibody has a higher affinity for the antigen when compared to the parental antibody.

If the antibody is for use as a therapeutic, it can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

The antibody can be conjugated to an effector moiety such as a small molecule toxin or a radionuclide using standard in vitro conjugation chemistries. If the effector moiety is a polypeptide, the antibody can be chemically conjugated to the effector or joined to the effector as a fusion protein. Construction of fusion proteins is within ordinary skill in the art.

In certain embodiments, the protein (e.g., antibody) of the present disclosure is not substantially internalized by a FLT3-expressing cell. A low level of internalization may improve the pharmacokinetics of the protein, thereby reducing the dose required to engage FLT3-expressing target cells with effector cells (e.g., NK cells). Internalization can be measured by any method known in the art, e.g., the methods described in Example 7 of the present disclosure. For example, in certain embodiments, internalization of the protein by ROH or EOL-1 cells is lower than 10%, 15%, 20%, 25%, 30%, 35%, 40%, 45%, or 50% after a two-hour incubation, as assessed by the methods disclosed herein.

### CAR T Cells, FLT3/CD3-Directed Bispecific T-Cell Engagers, Immunocytokines, Antibody-Drug Conjugates, and Immunotoxins

Another aspect of the present invention provides a molecule or complex comprising an antigen-binding site that binds FLT3 as disclosed herein. Exemplary molecules or complexes include but are not limited to chimeric antigen receptors (CARs), T-cell engagers (e.g., FLT3/CD3-directed bispecific T-cell engagers), immunocytokines, antibody-drug conjugates, and immunotoxins.

Any antigen-binding site that binds FLT3 as disclosed herein can be used. In certain embodiments, the VH, VL, and/or CDR sequences of the antigen-binding site that binds FLT3 are provided in Table 1. In certain embodiments, the antigen-binding site that binds FLT3 is an scFv. In certain embodiments, the scFv comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, and 82. In certain embodiments, the scFv comprises an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, and 82.

In certain embodiments, the antigen-binding site that binds FLT3 comprises a heavy chain variable domain comprising CDR1, CDR2, and CDR3 sequences represented by the amino acid sequences of SEQ ID NOs: 11, 4, and 5, respectively; and a light chain variable domain comprising CDR1, CDR2, and CDR3 sequences represented by the amino acid sequences of SEQ ID NOs: 6, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:37; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:38. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:40 or SEQ ID NO:39.

### Chimeric Antigen Receptors (CARs)

In certain embodiments, the present invention provides a FLT3-targeting CAR comprising an antigen-binding site that binds FLT3 as disclosed herein (see, e.g., Table 1). The FLT3-targeting CAR can comprise an Fab fragment or an scFv.

The term “chimeric antigen receptor” or alternatively a “CAR” refers to a recombinant polypeptide construct comprising at least an extracellular antigen binding domain, a transmembrane domain and an intracellular signaling domain comprising a functional signaling domain derived from a stimulatory molecule (also referred to herein as a “primary signaling domain”).

Accordingly, in certain embodiments, the CAR comprises an extracellular antigen-binding site that binds FLT3 as disclosed herein, a transmembrane domain, and an intracellular signaling domain comprising a primary signaling domain. In certain embodiments, the CAR further comprises one or more functional signaling domains derived from at least one costimulatory molecule (also referred to as a “costimulatory signaling domain”).

In one embodiment, the CAR comprises a chimeric fusion protein comprising a FLT3-binding domain (e.g., FLT3-binding scFv domain) comprising CDR1, CDR2, and CDR3 of a heavy chain variable domain and CDR1, CDR2, and CDR3 of a light chain variable domain listed in Table 1 as an extracellular antigen binding domain, a transmembrane domain, and an intracellular signaling domain comprising a primary signaling domain. In one embodiment, the CAR comprises a chimeric fusion protein comprising a FLT3-binding domain (e.g., FLT3-binding scFv domain) comprising CDR1, CDR2, and CDR3 of a heavy chain variable domain and CDR1, CDR2, and CDR3 of a light chain variable domain listed in Table 1 as an extracellular antigen binding domain, a transmembrane domain and an intracellular signaling domain comprising a costimulatory signaling domain and a primary signaling domain. In one aspect, the CAR comprises a chimeric fusion protein comprising a FLT3-binding domain (e.g., FLT3-binding scFv domain) comprising CDR1, CDR2, and CDR3 of a heavy chain variable domain and CDR1, CDR2, and CDR3 of a light chain variable domain listed in Table 1 as an extracellular antigen binding domain, a transmembrane domain, and an intracellular signaling domain comprising two costimulatory signaling domains and a primary signaling domain. In one embodiment, the CAR comprises a chimeric fusion protein comprising a FLT3-binding domain comprising CDR1, CDR2, and CDR3 of a heavy chain variable domain and CDR1, CDR2, and CDR3 of a light chain variable domain listed in Table 1 as an extracellular antigen binding domain, a transmembrane domain, and an intracellular signaling domain comprising at least two costimulatory signaling domains and a primary signaling domain.

For example, in certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 11, 4, and 5, respectively; and a light chain variable domain comprising CDR1, CDR2, and CDR3 sequences represented by the amino acid sequences of SEQ ID NOs: 6, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:37; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:38. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:40 or SEQ ID NO:39.

With respect to the transmembrane domain, in various embodiments, the CAR is designed to comprise a transmembrane domain that is fused to the extracellular domain of the CAR. In one embodiment, the transmembrane domain is one that naturally is associated with one of the domains in the CAR. In some instances, the transmembrane domain can be selected or modified by amino acid substitution to avoid binding of such domains to the transmembrane domains of the same or different surface membrane proteins to minimize interactions with other members of the receptor complex. In another embodiment, the transmembrane domain is capable of homodimerization with another CAR on the CAR T cell surface. In another embodiment, the amino acid sequence of the transmembrane domain may be modified or substituted so as to minimize interactions with the binding domains of the native binding partner present in the same CAR T cell.

The transmembrane domain may be derived from any naturally occurring membrane-bound or transmembrane protein. In one embodiment, the transmembrane region is capable of signaling to the intracellular domain(s) whenever the CAR has bound to a target. In some embodiments, the transmembrane domain comprises the transmembrane region(s) of one or more proteins selected from the group consisting of TCR α chain, TCR β chain, TCR ζ chain, CD28, CD3ε, CD45, CD4, CD5, CD8, CD9, CD16, CD22, FLT3, CD37, CD64, CD80, CD86, CD134, CD137, and CD154. In some embodiments, the transmembrane domain comprises the transmembrane region(s) of one or more proteins selected from the group consisting of KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NKG2C.

The extracellular FLT3-binding domain (e.g., FLT3-binding scFv domain) domain can be connected to the transmembrane domain by a hinge region. A variety of hinges can be employed, including but not limited to the human Ig (immunoglobulin) hinge (e.g., an IgG4 hinge, an IgD hinge), a Gly-Ser linker, a (G₄S)₄ linker, a KIR2DS2 hinge, and a CD8α hinge.

The intracellular signaling domain of the CAR of the present invention is responsible for activation of at least one of the specialized functions of the immune cell (e.g., cytolytic activity or helper activity, including the secretion of cytokines, of a T cell) in which the CAR has been placed in. Thus, as used herein, the term “intracellular signaling domain” refers to the portion of a protein which transduces an effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain of the CAR comprises a primary signaling domain (i.e., a functional signaling domain derived from a stimulatory molecule) and one or more costimulatory signaling domains (i.e., functional signaling domains derived from at least one costimulatory molecule).

As used herein, the term “stimulatory molecule” refers to a molecule expressed by an immune cell, e.g., a T cell, an NK cell, or a B cell, that provide the cytoplasmic signaling sequence(s) that regulate activation of the immune cell in a stimulatory way for at least some aspect of the immune cell signaling pathway. In one embodiment, the signal is a primary signal that is initiated by, for instance, binding of a TCR/CD3 complex with an WIC molecule loaded with a peptide, and which leads to mediation of a T cell response, including, but not limited to, proliferation, activation, differentiation, and the like.

Primary signaling domains that act in a stimulatory manner may contain signaling motifs which are known as immunoreceptor tyrosine-based activation motifs or ITAMs. Examples of ITAM containing cytoplasmic signaling sequences that are of particular use in the present invention include those derived from CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12. In one embodiment, the primary signaling domain in any one or more CARs of the present invention comprises a cytoplasmic signaling sequence derived from CD3-zeta.

In some embodiments, the primary signaling domain is a functional signaling domain of TCR zeta, FcR gamma, FcR beta, CD3 gamma, CD3 delta, CD3 epsilon, CD5, CD22, CD79a, CD79b, CD66d, 4-1BB, and/or CD3-zeta. In an embodiment, the intracellular signaling domain comprises a functional signaling domain of CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and/or DAP12. In a particular embodiment, the primary signaling domain is a functional signaling domain of the zeta chain associated with the T cell receptor complex.

As used herein, the term “costimulatory molecule” refers to a cognate binding partner on a T cell that specifically binds with a costimulatory ligand, thereby mediating a costimulatory response by the T cell, such as, but not limited to, proliferation. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1, CD11a/CD18), CD2, CD7, CD258 (LIGHT), NKG2C, B7-H3, and a ligand that specifically binds with CD83, and the like. Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and a ligand that specifically binds with CD83. In some embodiments, the costimulatory signaling domain of the CAR is a functional signaling domain of a costimulatory molecule described herein, e.g., OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

As used herein, the term “signaling domain” refers to the functional portion of a protein which acts by transmitting information within the cell to regulate cellular activity via defined signaling pathways by generating second messengers or functioning as effectors by responding to such messengers.

The cytoplasmic signaling sequences within the cytoplasmic signaling portion of the CAR of the present invention may be linked to each other in a random or specified order. Optionally, a short oligo- or polypeptide linker, for example, between 2 and 10 amino acids in length may form the linkage.

Another aspect of the present invention provides a nucleic acid encoding a FLT3-targeting CAR disclosed herein. The nucleic acid is useful for expressing the CAR in an effector cell (e.g., T cell) by introducing the nucleic acid to the cell.

Modifications may be made in the sequence to create an equivalent or improved variant of the present invention, for example, by changing one or more of the codons according to the codon degeneracy table. A DNA codon degeneracy table is provided in Table 2.

TABLE 2
Amino Acid Codons

| | One | Three | |
|---|---|---|---|
| | letter | letter | |
| Amino Acids | code | code | Codons |
| Alanine | A | Ala | GCA GCC GCG GCU |
| Cysteine | C | Cys | UGC UGU |
| Aspartic acid | D | Asp | GAC GAU |
| Glutamic acid | E | Glu | GAA GAG |
| Phenylalanine | F | Phe | UUC UUU |
| Glycine | G | Gly | GGA GGC GGG GGU |
| Histidine | H | His | CAC CAU |
| Isoleucine | I | Iso | AUA AUC AUU |
| Lysine | K | Lys | AAA AAG |
| Leucine | L | Leu | UUA UUG CUA CUC CUG CUU |
| Methionine | M | Met | AUG |
| Asparagine | N | Asn | AAC AAU |
| Proline | P | Pro | CCA CCC CCG CCU |
| Glutamine | Q | Gln | CAA CAG |
| Arginine | R | Arg | AGA AGG CGA CGC CGG CGU |
| Serine | S | Ser | AGC AGU UCA UCC UCG UCU |
| Threonine | T | Thr | ACA ACC ACG ACU |
| Valine | V | Val | GUA GUC GUG GUU |
| Tryptophan | W | Trp | UGG |
| Tyrosine | Y | Tyr | UAC UAU |

In certain embodiments, the nucleic acid is a DNA molecule (e.g., a cDNA molecule). In certain embodiments, the nucleic acid further comprises an expression control sequence (e.g., promoter and/or enhancer) operably linked to the CAR coding sequence. In certain embodiments, the present invention provides a vector comprising the nucleic acid. The vector can be a viral vector (e.g., AAV vector, lentiviral vector, or adenoviral vector) or a non-viral vector (e.g., plasmid).

In certain embodiments, the nucleic acid is an RNA molecule (e.g., an mRNA molecule). A method for generating mRNA for use in transfection can involve in vitro transcription of a template with specially designed primers, followed by polyA addition, to produce an RNA construct containing 3′ and 5′ untranslated sequences, a 5′ cap and/or Internal Ribosome Entry Site (IRES), the nucleic acid to be expressed, and a polyA tail, typically 50-2000 bases in length. The RNA molecule can be further modified to increase translational efficiency and/or stability, e.g., as disclosed in U.S. Pat. Nos. 8,278,036; 8,883,506, and 8,716,465. RNA molecules so produced can efficiently transfect different kinds of cells.

In one embodiment, the nucleic acid encodes an amino acid sequence comprising a signal peptide at the amino-terminus of the CAR. Such signal peptide can facilitate the cell surface localization of the CAR when it is expressed in an effector cell, and is cleaved from the CAR during cellular processing. In one embodiment, the nucleic acid encodes an amino acid sequence comprising a signal peptide at the N-terminus of the extracellular FLT3-binding domain (e.g., FLT3-binding scFv domain).

RNA or DNA can be introduced into target cells using any of a number of different methods, for instance, commercially available methods which include, but are not limited to, electroporation, cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001)).

Another aspect of the present invention provides an immune effector cell expressing the FLT3-targeting CAR. Also provided is an immune effector cell comprising the nucleic acid encoding the FLT3-targeting CAR. The immune effector cells include but are not limited to T cells and NK cells. In certain embodiments, the T cell is selected from a CD8⁺ T cell, a CD4⁺ T cell, and an NKT cell. The T cell or NK cell can be a primary cell or a cell line.

The immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors, by methods known in the art. The immune effector cells can also be differentiated in vitro from a pluripotent or multipotent cell (e.g., a hematopoietic stem cell). In some embodiments, the present invention provides a pluripotent or multipotent cell (e.g., a hematopoietic stem cell) expressing the FLT3-targeting CAR (e.g., expressing the CAR on the plasma membrane) or comprising a nucleic acid disclosed herein.

In certain embodiments, the immune effector cells are isolated and/or purified. For example, regulatory T cells can be removed from a T cell population using a CD25-binding ligand. Effector cells expressing a checkpoint protein (e.g., PD-1, LAG-3, or TIM-3) can be removed by similar methods. In certain embodiments, the effector cells are isolated by a positive selection step. For example, a population of T cells can be isolated by incubation with anti-CD3/anti-CD28-conjugated beads. Other cell surface markers, such as IFN-7, TNF-α, IL-17A, IL-2, IL-3, IL-4, GM-CSF, IL-10, IL-13, granzyme B, and perforin, can also be used for positive selection.

Immune effector cells may be activated and expanded generally using methods known in the art, e.g., as described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publications Nos. 2006/0121005 and 2016/0340406. For example, in certain embodiments, T cells can be expanded and/or activated by contact with an anti-CD3 antibody and an anti-CD28 antibody, under conditions appropriate for stimulating proliferation of the T cells. The cells can be expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. Multiple cycles of stimulation may be desirable for prolonged cell culture (e.g., culture for a period of 60 days or more). In certain embodiments, the cell culture comprises serum (e.g., fetal bovine or human serum), interleukin-2 (IL-2), insulin, IFN-γ, IL-4, IL-7, GM-CSF, IL-10, IL-12, IL-15, TGFβ, TNF-α, or a combination thereof. Other additives for the growth of cells known to the skilled person, e.g., surfactant, plasmanate, and reducing agents such as N-acetyl-cysteine and 2-mercaptoethanol, can also be included in the cell culture. In certain embodiments, the immune effector cell of the present invention is a cell obtained from in vitro expansion.

Further embodiments of the FLT3-targeting CAR (e.g., regulatable CAR), nucleic acid encoding the CAR, and effector cells expressing the CAR or comprising the nucleic acid are provided in U.S. Pat. Nos. 7,446,190 and 9,181,527, U.S. Patent Application Publication Nos. 2016/0340406 and 2017/0049819, and International Patent Application Publication No. WO2018/140725.

### FLT3/CD3-Directed Bispecific T-Cell Engagers

In certain embodiments, the present invention provides a FLT3/CD3-directed bispecific T-cell engager comprising an antigen-binding site that binds FLT3 disclosed herein. In certain embodiments, the FLT3/CD3-directed bispecific T-cell engager comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, and 82. In certain embodiments, the cytokine is connected to the Fc domain directly or via a linker.

In certain embodiments, the FLT3/CD3-directed bispecific T-cell engager further comprises an antigen-binding site that binds CD3. Exemplary antigen-binding sites that bind CD3 are disclosed in International Patent Application Publication Nos. WO2014/051433 and WO2017/097723.

Another aspect of the present invention provides a nucleic acid encoding at least one polypeptide of the FLT3/CD3-directed bispecific T-cell engager, wherein the polypeptide comprises an antigen-binding site that binds FLT3. In certain embodiments, the nucleic acid further comprises a nucleotide sequence encoding a signal peptide that, when expressed, is at the N-terminus of one or more of the polypeptides of the FLT3/CD3-directed bispecific T-cell engager. Also provided is a vector (e.g., a viral vector) comprising the nucleic acid, a producer cell comprising the nucleic acid or vector, and a producer cell expressing the FLT3/CD3-directed bispecific T-cell engager.

### Immunocytokines

In certain embodiments, the present invention provides an immunocytokine comprising an antigen-binding site that binds FLT3 disclosed herein and a cytokine. Any cytokine (e.g., pro-inflammatory cytokines) known in the art can be used, including but not limited to IL-2, IL-4, IL-10, IL-12, IL-15, TNF, IFNα, IFNγ, and GM-CSF. More exemplary cytokines are disclosed in U.S. Pat. No. 9,567,399. In certain embodiments, the antigen-binding site is connected to the cytokine by chemical conjugation (e.g., covalent or noncovalent chemical conjugation). In certain embodiments, the antigen-binding site is connected to the cytokine by fusion of polypeptide. The immunocytokine can further comprise an Fc domain connected to the antigen-binding site that binds FLT3. In certain embodiments, the immunocytokine comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, and 82. In certain embodiments, the cytokine is connected to the Fc domain directly or via a linker.

Another aspect of the present invention provides a nucleic acid encoding at least one polypeptide of the immunocytokine, wherein the polypeptide comprises an antigen-binding site that binds FLT3. In certain embodiments, the nucleic acid further comprises a nucleotide sequence encoding a signal peptide that, when expressed, is at the N-terminus of one or more of the polypeptides of the immunocytokine. Also provided is a vector (e.g., a viral vector) comprising the nucleic acid, a producer cell comprising the nucleic acid or vector, and a producer cell expressing the immunocytokine.

### Antibody-Drug Conjugates

In certain embodiments, the present invention provides an antibody-drug conjugate comprising an antigen-binding site that binds FLT3 disclosed herein and a cytotoxic drug moiety. Exemplary cytotoxic drug moieties are disclosed in International Patent Application Publication Nos. WO2014/160160 and WO2015/143382. In certain embodiments, the cytotoxic drug moiety is selected from auristatin, N-acetyl-γ calicheamicin, maytansinoid, pyrrolobenzodiazepine, and SN-38. The antigen-binding site can be connected to the cytotoxic drug moiety by chemical conjugation (e.g., covalent or noncovalent chemical conjugation). In certain embodiments, the antibody-drug conjugate further comprises an Fc domain connected to the antigen-binding site that binds FLT3. In certain embodiments, the antibody-drug conjugate comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, and 82. In certain embodiments, the cytotoxic drug moiety is connected to the Fc domain directly or via a linker.

### Immunotoxins

In certain embodiments, the present invention provides an immunotoxin comprising an antigen-binding site that binds FLT3 disclosed herein and a cytotoxic peptide moiety. Any cytotoxic peptide moiety known in the art can be used, including but not limited to ricin, Diphtheria toxin, and Pseudomonas exotoxin A. More exemplary cytotoxic peptides are disclosed in International Patent Application Publication Nos. WO2012/154530 and WO2014/164680. In certain embodiments, the cytotoxic peptide moiety is connected to the protein by chemical conjugation (e.g., covalent or noncovalent chemical conjugation). In certain embodiments, the cytotoxic peptide moiety is connected to the protein by fusion of polypeptide. The immunotoxin can further comprise an Fc domain connected to the antigen-binding site that binds FLT3. In certain embodiments, the immunotoxin comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, and 82. In certain embodiments, the cytotoxic peptide moiety is connected to the Fc domain directly or via a linker.

Another aspect of the present invention provides a nucleic acid encoding at least one polypeptide of the immunotoxin, wherein the polypeptide comprises an antigen-binding site that binds FLT3. In certain embodiments, the nucleic acid further comprises a nucleotide sequence encoding a signal peptide that, when expressed, is at the N-terminus of one or more of the polypeptides of the immunotoxin. Also provided is a vector (e.g., a viral vector) comprising the nucleic acid, a producer cell comprising the nucleic acid or vector, and a producer cell expressing the immunotoxin.

### II. Therapeutic Compositions and Their Use

The present invention provides methods for treating cancer using a protein, conjugate, or cells comprising an antigen-binding site disclosed herein and/or a pharmaceutical composition described herein. The methods may be used to treat a variety of cancers which express FLT3 by administering to a patient in need thereof a therapeutically effective amount of a protein, conjugate, or cells comprising an antigen-binding site disclosed herein.

The therapeutic method can be characterized according to the cancer to be treated. For example, in certain embodiments, the cancer is a hematologic malignancy or leukemia. In certain embodiments, the cancer is acute myeloid leukemia (AML), acute lymphoblastic leukemia (ALL), myelodysplasia, myelodysplastic syndromes, acute T-lymphoblastic leukemia, or acute promyelocytic leukemia, chronic myelomonocytic leukemia, or myeloid blast crisis of chronic myeloid leukemia.

In certain embodiments, the AML is a minimal residual disease (MRD). In certain embodiments, the MRD is characterized by the presence or absence of a mutation selected from FLT3-ITD ((Fms-like tyrosine kinase 3)-internal tandem duplications (ITD)), NPM1 (Nucleophosmin 1), DNMT3A (DNA methyltransferase gene DNMT3A), and IDH (Isocitrate dehydrogenase 1 and 2 (IDH1 and IDH2)). In certain embodiments, the MDS is selected from MDS with multilineage dysplasia (MDS-MLD), MDS with single lineage dysplasia (MDS-SLD), MDS with ring sideroblasts (MDS-RS), MDS with excess blasts (MDS-EB), MDS with isolated del(5q), and MDS, unclassified (MDS-U). In certain embodiments, the MDS is a primary MDS or a secondary MDS.

In certain embodiments, the ALL is selected from B-cell acute lymphoblastic leukemia (B-ALL) and T-cell acute lymphoblastic leukemia (T-ALL). In certain embodiments, the MPN is selected from polycythaemia vera, essential thrombocythemia (ET), and myelofibrosis. In certain embodiments, the non-Hodgkin lymphoma is selected from B-cell lymphoma and T-cell lymphoma. In certain embodiments, the lymphoma is selected from chronic lymphocytic leukemia (CLL), lymphoblastic lymphoma (LPL), diffuse large B-cell lymphoma (DLBCL), Burkitt lymphoma (BL), primary mediastinal large B-cell lymphoma (PMBL), follicular lymphoma, mantle cell lymphoma, hairy cell leukemia, plasma cell myeloma (PCM) or multiple myeloma (MM), mature T/NK neoplasms, and histiocytic neoplasms.

In certain embodiments, the cancer is a solid tumor. In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, bilary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In certain other embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

The cancer to be treated can be characterized according to the presence of a particular antigen expressed on the surface of the cancer cell. In certain embodiments, the cancer cell can express one or more of the following in addition to FLT3: CD2, CD19, CD20, CD30, CD38, CD40, CD52, CD70, EGFR/ERBB1, IGF1R, HER3/ERBB3, HER4/ERBB4, MUC1, TROP2, cMET, SLAMF7, PSCA, MICA, MICB, TRAILR1, TRAILR2, MAGE-A3, B7.1, B7.2, CTLA4, and PD1.

In embodiments of the present invention, the cancer to be treated is selected from acute myeloid leukemia (AML), myelodysplastic syndrome (MDS), acute lymphoblastic leukemia (ALL), myeloproliferative neoplasms (MPNs), lymphoma, non-Hodgkin lymphomas, and classical Hodgkin lymphoma.

In some embodiments of the present invention, the cancer to be treated is AML. In some embodiments of the present invention, the AML is selected from undifferentiated acute myeloblastic leukemia, acute myeloblastic leukemia with minimal maturation, acute myeloblastic leukemia with maturation, acute promyelocytic leukemia (APL), acute myelomonocytic leukemia, acute myelomonocytic leukemia with eosinophilia, acute monocytic leukemia, acute erythroid leukemia, acute megakaryoblastic leukemia (AMKL), acute basophilic leukemia, acute panmyelosis with fibrosis, and blastic plasmacytoid dendritic cell neoplasm (BPDCN). In some embodiments of the present invention, the AML is characterized by expression of CLL-1 on the AML leukemia stem cells (LSCs). In some embodiments of the present invention, the LSCs in an AML subject further express a membrane marker selected from CD34, CD38, CD123, TIM3, CD25, CD32, and CD96. In some embodiments of the present invention, the AML is characterized as a minimal residual disease (MRD). In some embodiments of the present invention, the MRD of AML is characterized by the presence or absence of a mutation selected from FLT3-ITD ((Fms-like tyrosine kinase 3)-internal tandem duplications (ITD)), NPM1 (Nucleophosmin 1), DNMT3A (DNA methyltransferase gene DNMT3A), and IDH (Isocitrate dehydrogenase 1 and 2 (IDH1 and IDH2)).

In certain embodiments of the present invention, the cancer is MDS selected from MDS with multilineage dysplasia (MDS-MLD), MDS with single lineage dysplasia (MDS-SLD), MDS with ring sideroblasts (MDS-RS), MDS with excess blasts (MDS-EB), MDS with isolated del(5q), and MDS, unclassified (MDS-U).

It is contemplated that the protein, conjugate, cells, and/or pharmaceutical compositions of the present disclosure can be used to treat a variety of cancers, not limited to cancers in which the cancer cells express FLT3. For example, in certain embodiments, the protein, conjugate, cells, and/or pharmaceutical compositions disclosed herein can be used to treat cancers that are associated with FLT3-expressing immune cells. FLT3 is expressed on many myeloid lineages, and tumor-infiltrating myeloid cells (e.g., tumor-associated macrophages) may contribute to cancer progression and metastasis. Therefore, the methods disclosed herein may be used to treat a variety of cancers in which FLT3 is expressed, whether on cancer cells or on immune cells.

### III. Combination Therapy

Another aspect of the present invention provides for combination therapy. Proteins, conjugates, and cells comprising an antigen-binding site described herein can be used in combination with additional therapeutic agents to treat the cancer.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins of the present invention can also be used as an adjunct to surgical removal of the primary lesion.

The amount of the protein, conjugate, or cells disclosed herein and the additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a protein, conjugate, or cell disclosed herein may be administered during a time when the additional therapeutic agent(s) exerts its prophylactic or therapeutic effect, or vice versa.

### IV. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In one aspect, the present disclosure provides a formulation of a protein, which contains a FLT3-binding site described herein, and a pharmaceutically acceptable carrier.

In certain embodiments, the pharmaceutical composition includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:1, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:2. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:9, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:10. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:13, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:10. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:17, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:10. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:9, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:22. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:9, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:26. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:9, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:30. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:9, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:34. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:37, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:38. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:41, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:42. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:45, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:42. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:49, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:42. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:60, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:61. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:68, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:69. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:72, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:73. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:76, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:77. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:85, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:90. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:14, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:94. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:95, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:96. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:104, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:105. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:107, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:108. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:115, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:116. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:123, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:124. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:125, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:126. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:131, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:83.

The composition can be formulated for use in a variety of drug delivery systems. One or more physiologically acceptable excipients or carriers can be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

For example, this present disclosure could exist in an aqueous pharmaceutical formulation including a therapeutically effective amount of the protein in a buffered solution forming a formulation. Aqueous carriers can include sterile water for injection (SWFI), bacteriostatic water for injection (BWFI), a pH buffered solution (e.g. phosphate-buffered saline), sterile saline solution, Ringer's solution or dextrose solution. In certain embodiments, an aqueous formulation is prepared including the protein disclosed herein in a pH-buffered solution. The pH of the preparations typically will be between 3 and 11, more preferably between 5 and 9 or between 6 and 8, and most preferably between 7 and 8, such as 7 to 7.5. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g. 1.53 mg/mL), about 0.9 mg/mL of sodium dihydrogen phosphate dihydrate (e.g., 0.86), and about 6.2 mg/mL of sodium chloride (e.g., 6.165 mg/mL). In certain embodiments, the buffer system includes 1-1.5 mg/mL of citric acid, 0.25 to 0.5 mg/mL of sodium citrate, 1.25 to 1.75 mg/ml of disodium phosphate dihydrate, 0.7 to 1.1 mg/mL of sodium dihydrogen phosphate dihydrate, and 6.0 to 6.4 mg/mL of sodium chloride. The pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In some embodiments, the formulation include an aqueous carrier, which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to about 15 mg/mL. In certain embodiments, the concentration of mannitol may be about 10 to about 14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative, which is added to the formulations herein to reduce bacterial action. The addition of a preservative may, for example, facilitate the production of a multi-use (multiple-dose) formulation.

In some embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

Deamidation is a common product variant of peptides and proteins that may occur during fermentation, harvest/cell clarification, purification, drug substance/drug product storage and during sample analysis. Deamidation is the loss of NH3 from a protein forming a succinimide intermediate that can undergo hydrolysis. The succinimide intermediate results in a 17 dalton mass decrease of the parent peptide. The subsequent hydrolysis results in an 18 dalton mass increase. Isolation of the succinimide intermediate is difficult due to instability under aqueous conditions. As such, deamidation is typically detectable as 1 dalton mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation. In certain embodiments, the liquid formulation of the present disclosure may be preserved under conditions of pH and humidity to prevent deamination of the protein product.

In some embodiment, the formulation is a lyophilized formulation. In certain embodiments, the formulation is freeze-dried (lyophilized) and contained in about 12-60 vials. In certain embodiments, the formulation is freeze-dried and 45 mg of the freeze-dried formulation may be contained in one vial. In certain embodiments, the about 40 mg-about 100 mg of freeze-dried formulation is contained in one vial. In certain embodiments, freeze dried formulation from 12, 27, or 45 vials are combined to obtained a therapeutic dose of the protein in the intravenous drug formulation. The formulation may be a liquid formulation. In some embodiments, a liquid formulation is stored as about 250 mg/vial to about 1000 mg/vial. In certain embodiments, the liquid formulation is stored as about 600 mg/vial. In certain embodiments, the liquid formulation is stored as about 250 mg/vial.

In some embodiments, the lyophilized formulation includes the proteins described herein and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative. The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide. Before lyophilization, the pH of the solution containing the protein of the present disclosure may be adjusted between 6 to 8. In certain embodiments, the pH range for the lyophilized drug product may be from 7 to 8.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

In certain embodiments, the lyophilized protein product is constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer's solution or dextrose solution. In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution. In certain embodiments, the lyophilized protein product of the instant disclosure is constituted to about 4.5 mL water for injection and diluted with 0.9% saline solution (sodium chloride solution).

The protein compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The resulting compositions in solid form may be packaged in multiple single dose units, each containing a fixed amount of the above-mentioned agent or agents. The composition in solid form can also be packaged in a container for a flexible quantity.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica. Chimica. Acta. 308: 43-53, 2001; Steimer et al., Clinica. Chimica. Acta. 308: 33-41, 2001).

In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight. Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above describes multiple aspects and embodiments of the present invention. The patent application specifically contemplates all combinations and permutations of the aspects and embodiments.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following examples are merely illustrative and are not intended to limit the scope or content of the invention in any way.

### Example 1. Characterization of Supernatants of Selected Hybridoma Clones

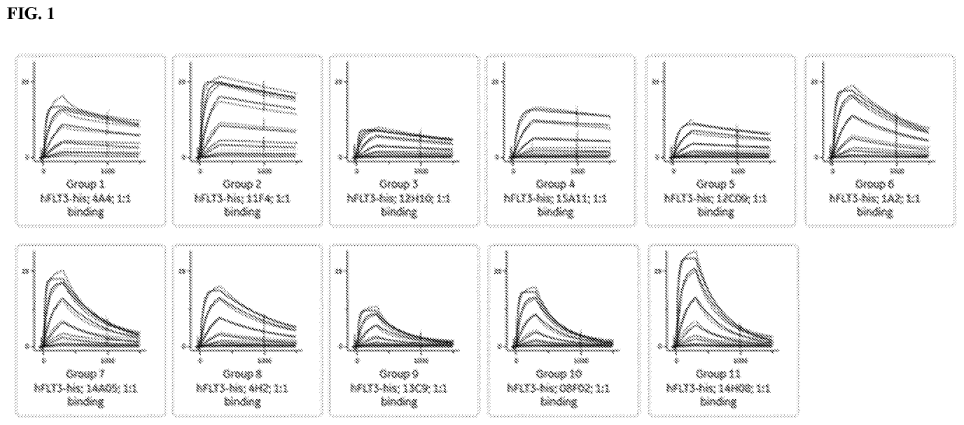

FLT3-specific antibodies were generated by immunizing mice with hFLT3-His fusion protein. Supernatants of 228 hybridomas were assessed for FLT3 binding by enzyme-linked immunosorbent assay (ELISA), and 96 hybridomas bound noncovalently to hFLT3-His protein. Eleven clones were selected based on preliminary Bio-layer Interferometry (BLI) binding affinity estimations, binding to human and cynomolgus monkey cell expressing FLT3, and diversity of epitopes. The ability of these 11 clones to bind hFLT3-His was further analyzed by high resolution surface plasmon resonance (SPR). The experiment was performed at 37° C. to mimic physiological temperature using a Biacore 8K instrument. Biacore sensorgrams and kinetic parameters are presented in Table 4 and raw data and fits are shown in FIG. 1. Seven out of eleven hybridomas bound with K_{D} less than 10 nM, and five display slow dissociation rate constant (k_{d}<5×10⁻⁴ s⁻¹).

Binning of hybridoma fusions with reference mAbs was performed by BLI using OctetRed384 (ForteBio). Briefly, hybridoma supernatants were loaded onto anti-mouse IgG capture sensor tips for 15 minutes and equilibrated for 5 minutes in PB SF. Sensors were dipped into 200 nM hFLT3-His and allowed to associate for 180 seconds followed by dipping into 100 nM control IgGs or 200 nM FTL3-ligand solution. The increase in response units indicated the hybridoma was a non-competitor to the reference mAb, while no increase in signal indicated that hybridoma did compete with the reference mAb. FL23 (Amgen) and FL39 (Amgen) bind to Domain 1. EB10 (ImClone), a known FLT3-ligand blocker, binds to Domain 3. FL61 (Amgen) also binds to domain 3, but is not a FLT3-ligand blocker. 4G8 (Synimmune) binds to Domain 4. NC7 (Imclone) binds to Domain 5. The VH and VL sequences of these reference antibodies are provided in Table 3.

TABLE 3
Reference antibodies

| α-FLT3 mAb | VH | VL |
|---|---|---|
| 4G8 | QVQLQQPGAELVKPGASLKLSCKS | DIVLTQSPATLSVTPGDSVSLS |
| (Synimmune), | SGYTFTSYWMHWVRQRPGHGLE | CRASQSISNNLHWYQQKSHES |
| disclosed in U.S. | WIGEIDPSDSYKDYNQKFKDKATL | PRLLIKYASQSISGIPSRFSGSG |
| Application | TVDRSSNTAYMHLSSLTSDDSAVY | SGTDFTLSINSVETEDFGVYFC |
| Publication No. | YCARAITTTPFDFWGQGTTLTVSS | QQSNTWPYTFGGGTKLEIK |
| 2015/0119555A1 | (SEQ ID NO: 135) | (SEQ ID NO: 136) |
| EB10 | EVQLVQSGAEVKKPGASVKVSCK | DVVMTQSPLSLPVTPGEPASIS |
| (ImClone/Lilly), | ASGYTFTSYYMHWVRQAPGQGLE | CRSSQSLLHSNGNNYLDWYL |
| disclosed in U.S. | WMGIINPSGGSTSYAQKFQGRVT | QKPGQSPQLLIYLGSNRASGV |
| Application | MTRDTSTSTVYMELSSLRSEDTAV | PDRFSGSGSDTDFTLQISRVEA |
| Publication No. | YYCARGVGAHDAFDIWGQGTTVT | EDVGVYYCMQGTHPAISFGQ |
| 2011/0008355A1 | VSS (SEQ ID NO: 137) | GTRLEIK (SEQ ID NO: 138) |
| NC7 | EVQLVQSGAEVKKPGSSVKVSCK | DIQMTQSPSSLSASVGDRVTIT |
| (Imclone/Lilly), | ASGGTFSSYAISWVRQAPGQGLE | CRASQSISSYLNWYQQKPGK |
| disclosed in U.S. | WMGGIIPIFGTANYAQKFQGRVTI | APKLLIYAASSLQSGVPSRFSG |
| Application | TADKSTSTAYMELSSLRSEDTAVY | SGSGTDFTLTISSLQPEDLATY |
| Publication No. | YCATFALFGFREQAFDIWGQGTTV | YCQQSYSTPFTFGPGTKVDIK |
| 2011/0008355A1 | TVSS (SEQ ID NO: 139) | (SEQ ID NO: 140) |
| FL23 (Amgen), | QVTLKESGPALVKPTETLTLTCTV | DIQMTQSPSSLSASVGDRVTIT |
| disclosed in U.S. | SGFSFRNARMGVSWIRQPPGKALE | CRASQDIGYDLGWYQQKPGK |
| Application | WLAHIFSNDEKSYSTSLKSRLTISK | APKRLIYAASTLQSGVPSRFS |
| Publication No. | DTSKSQVVLTLTNMDPVDTATYF | GSGSGTEFTLIISSLQPEDFAT |
| 2017/0037149A1 | CARMPEYSSGWSGAFDIWGQGTM | YYCLQHNSFPWTFGQGTKVEI |
| | VTVSS (SEQ ID NO: 141) | K (SEQ ID NO: 142) |
| FL39 (Amgen), | QVTLKESGPTLVKPTETLTLTCTLS | DIQMTQSPSSLSASVGDRVTIT |
| disclosed in U.S. | GFSLNNARMGVSWIRQPPGKCLE | CRASQGIRNDLGWYQQKPGK |
| Application | WLAHIFSNDEKSYSTSLKNRLTISK | APKRLIYAASTLQSGVPSRFS |
| Publication No. | DSSKTQVVLTMTNVDPVDTATYY | GSGSGTEFTLTISSLQPEDFAT |
| 2017/0037149A1 | CARIVGYGSGWYGFFDYWGQGTL | YYCLQHNSYPLTFGCGTKVEI |
| | VTVSS (SEQ ID NO: 143) | K (SEQ ID NO: 144) |
| FL61 (Amgen), | QVQLVESGGGVVQPGRSLRLSCA | DIQMTQSPSSLSASVGDRVTIT |
| disclosed in U.S. | ASGFTFSSYGMHWVRQAPGKGLE | CRASQSISSYLNWYQQKPGK |
| Application | WVAVISYDGSNEFYADSVKGRFTI | APKLLIYAASSLQSGVPSRFSG |
| Publication No. | SRDNSKNTLYLQMNSLRAEDTAV | SGSGTEFTLTISSLQPEDFATY |
| 2017/0037149A1 | YYCARGGEITMVRGVIGYYYYGM | YCLQHNSYPLTFGGGTKVEIK |
| | DVWGQGTTVTVSS | (SEQ ID NO: 146) |
| | (SEQ ID NO: 145) | |

It was observed that antibodies produced from five of the hybridomas, namely 4A4, 11F4, 1A2, 4H2, and 13C9, did not compete with any of the reference antibodies for binding to hFLT3-His. Cross-reactivity with cynomolgus monkey FLT3 (cFLT3) was evaluated by measuring the binding of the antibodies to isogenic RMA cells expressing cFLT3.

Briefly, RMA cells were transducted with a retroviral vector encoding cFLT3 or human FLT3 (hFLT3). Binding of the α-FLT3 mAbs from crude hybridoma harvests to the hFLT3 or cFLT3 isogenic cell lines, as well as FLT3+ cancer cell lines, was performed as follows. 100,000 RMA, REH or SEM cells were added per well of a 96 well round bottom plate. Cells were spun down and the pellet was gently dissociated by vortexing. 50 μL of Zombie live/dead dye (PBS+1:2000 dye) were added per well and incubated in the dark at room temperature for 20 minutes. Cells were washed with 200 μL of FACS buffer (PBS+2% FBS). 50 μL of hybridoma supernatants were added to the washed cells and the mixtures were incubated for 30 minutes on ice in the dark. Cells were washed once and then 50 μL of anti-mouse Fc-PE secondary reagent (1:200 dilution) were added and incubated for 20 minutes on ice in the dark. Cells were washed and fixed with 50 μL of 4% paraformaldehyde for 15 minutes on ice. Cells were washed again and then resuspended in 200 μL FACS buffer and stored at 4° C. until ready for acquisition. The samples were run on BD FACSCelesta equipped with an HTS (high throughput sampler).

The binding affinities of the hybridoma supernatants to REH cancer cells (ATCC catalog number CRL-8286), a human ALL cell line reported to express FLT3, were also measured. As shown in Table 4, most of the clones displayed binding affinity to cancer cells expressing hFLT3 and cross-reactivity with cFLT3. Cynomolgus monkey FLT3 binding data for 14A5 and 15A11 were not collected.

TABLE 4
Kinetic parameters and affinities of FLT3-His binding
to the antibodies produced from candidate hybridomas

| | | SPR at 37° C. | | | Cell Binding MFI | | |
|---|---|---|---|---|---|---|---|
| Test | Binning | | | K_{D} | RMA- | RMA- | |
| articles | profile | kₐ (1/Ms) | k_{d} (1/s) | (nM) | hFLT3 | cFLT3 | REH |
| 4A4 | unique | 3.38 × 10⁵ | 3.35 × 10⁻⁴ | 1.0 | 1493 | 2002 | 2002 |
| 11F4 | unique | 1.73 × 10⁵ | 1.88 × 10⁻⁴ | 1.1 | 305 | 495 | 495 |
| 12H10 | 4G8 | 1.74 × 10⁵ | 3.43 × 10⁻⁴ | 2.0 | 544 | 696 | 696 |
| 15A11 | EB10 | 4.99 × 10⁵ | 1.17 × 10⁻⁴ | 2.3 | 332 | n/a | n/a |
| 12C9 | FL23 | 5.67 × 10⁴ | 3.11 × 10⁻⁴ | 5.4 | 1020 | 3937 | 664 |
| 1A2 | unique | 1.14 × 10⁵ | 7.49 × 10⁻⁴ | 6.5 | 238 | 461 | 461 |
| 14A5 | FL23 | 1.70 × 10⁵ | 1.49 × 10⁻³ | 8.7 | 1005 | n/a | 2071 |
| 4H2 | unique | 8.05 × 10⁴ | 9.18 × 10⁻⁴ | 11 | 570 | 1017 | 1017 |
| 13C9 | unique | 2.12 × 10⁵ | 3.02 × 10⁻³ | 14 | 546 | 834 | 834 |
| 8F02 | FL23 | 1.67 × 10⁵ | 2.80 × 10⁻³ | 17 | 829 | 2729 | 2271 |
| 14H08 | FL23 | 1.29 × 10⁵ | 2.40 × 10⁻³ | 19 | 959 | 3074 | 1776 |

### Example 2. Analysis of Purified Anti-FLT3 Murine Antibodies

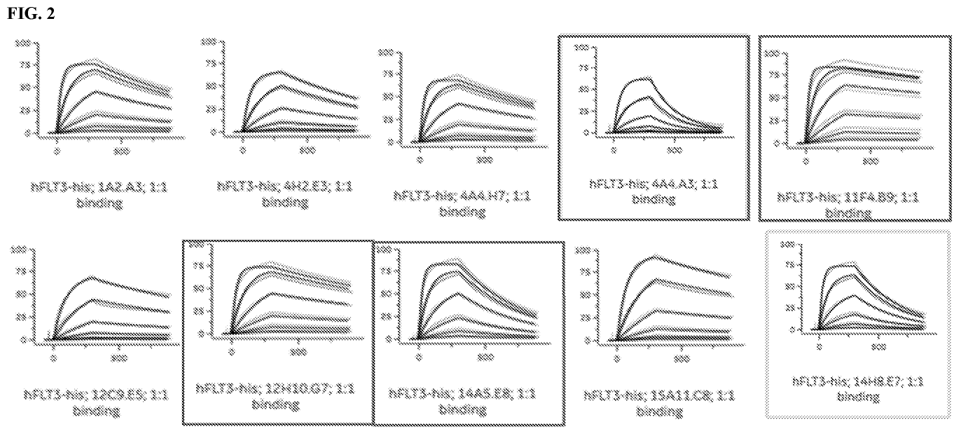

Based on the above presented analysis, eight hybridomas (4A4, 11F4, 12H10, 15A11, 12C09, 1A2, 14A5, 4H2) were selected for subcloning and sequencing. Two subclones from each parental hybridoma were produced and analyzed. Sequences from each hybridoma were determined to be unique. Each subclone was purified from the hybridoma culture, and binding to hFLT3-His was confirmed by SPR as shown in FIG. 2. Kinetic constants and binding affinities of hFLT3 to purified murine subcloned mAbs are shown in Table 5. Binning with reference antibodies was conducted using the method described in Example 1, and four antibodies, namely 4A4.A3, 11F4.B9, 1A2.A3, and 4H2.E3, did not compete with any of the reference antibodies for binding to hFLT3-His.

TABLE 5
Kinetic parameters and affinities of hFLT3
binding to purified murine subclones

| Test articles | kₐ (1/Ms) | k_{d} (1/s) | K_{D}, (nM) |
|---|---|---|---|
| 1A2.A3 | 1.1 × 10⁵ | 8.9 × 10⁻⁴ | 8.5 |
| 4A4.A3 | 1.1 × 10⁵ | 8.2 × 10⁻⁴ | 7.3 |
| 4H2.E3 | 5.7 × 10⁴ | 1.0 × 10⁻³ | 17.6 |
| 11F4.B9 | 1.5 × 10⁵ | 2.5 × 10⁻⁴ | 1.7 |
| 12C9.E5 | 3.4 × 10⁴ | 6.3 × 10⁻⁴ | 18.7 |
| 12H10.G7 | 1.0 × 10⁵ | 5.5 × 10⁻⁴ | 5.4 |
| 14A5.E8 | 1.3 × 10⁵ | 1.9 × 10⁻³ | 15.1 |
| 15A11.C8 | 4.5 × 10⁴ | 4.8 × 10⁻⁴ | 10.5 |

Cell binding of the purified subcloned mAbs was confirmed with isogenic human and cynomolgus monkey FLT3 expressing RMA cell lines. With the exception of 12C9.E5, all clones bound to cell surface expressed human and cynomolgus monkey FLT3 (Table 6). Similarly, all subclones bound with high affinity to SEM (DSMZ catalog number ACC 546), a human ALL cell line reported to express FLT3.

TABLE 6
Cell binding confirmation of purified mouse mAbs to
human and cynomolgus monkey FLT3 RMA cell lines

| | RMA- | | RMA- | | | |
|---|---|---|---|---|---|---|
| | hFLT3 | RMA- | cFLT3 | RMA- | SEM | |
| Test | EC50 | hFLT3 | EC50 | cFLT3 | EC50 | SEM |
| articles | (nM) | Max MFI | (nM) | Max MFI | (nM) | Max MFI |
| 1A2.A3 | 0.80 | 499 | 1.82 | 2834 | 5.47 | 1361 |
| 4A4.A3 | 0.72 | 1021 | 1.07 | 5566 | 3.29 | 2352 |
| 4H2.E3 | 0.66 | 696 | 1.56 | 3454 | 7.57 | 1510 |
| 11F4.B9 | 0.53 | 493 | 1.23 | 2589 | 2.43 | 1141 |
| 12C9.E5 | NB* | NB | NB | NB | NB | NB |
| 12H10.G7 | 0.36 | 1136 | 0.94 | 5262 | 3.20 | 2831 |
| 14A5.E8 | ~2.07 | 415 | 1.25 | 1779 | ~1.07 | 1956 |
| 15A11.C8 | 0.41 | 1406 | 0.82 | 6512 | ~1.13 | 3861 |

### Example 3. Ligand Blocking Properties of Selected Anti-FLT3 Murine Antibodies

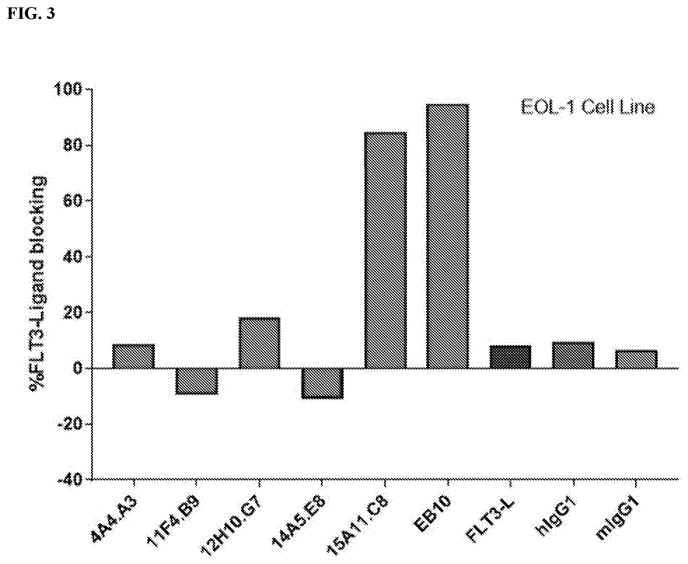

This Example was designed to characterize the ability of selected anti-FLT3 murine antibodies to block FLT3 interactions with FLT3-ligand. The ability of α-FLT3 mAbs to bind FLT3-expressing EOL-1 cancer cells (DSMZ catalog number ACC 386) was tested before and after the addition of saturating concentrations of soluble FLT3-ligand. For each antibody, its percentage of ligand blocking value was calculated as the decrease in mAb binding signal obtained in the presence of FLT3-ligand relative to that obtained in the absence of FLT3-ligand indicated. Known FLT3-ligand blocker EB10 mAb was used as a positive control. As shown in FIG. 3, the 12H10.G7, 11F4.B9 and 4A4.A3, 14A5.E8 antibodies did not interfere with binding of FLT3 to FLT3-ligand, whereas the 15A11.C8 antibody blocked the binding of FLT3-ligand to FLT3.

### Example 4. Putative Sequence Liability Analysis

Potential sequence liabilities in CDRs (identified under Chothia) of the 12H10.G7, 11F4.B9 and 4A4.A3, 14A5.E8 antibodies were examined. The following potential liabilities were considered: M (potential oxidation site); NG, NS and NT sequence motif (potential deamidation site); DG, DS and DT sequence motif (potential isomerization site); DP sequence motif (potential site for chemical hydrolysis). The results are summarized in Table 7.

TABLE 7
Putative sequence liabilities in the CDRs of selected murine mAbs

| | | location of sequence |
|---|---|---|
| Clone ID | Potential sequence liability motif | liability motif |
| 12H10.G7 | DS (isomerization site) | CDRH3 |
| 14A5.E8 | M (oxidation site) | CDRL1 |
| 11F4.B9 | M (oxidation site), NS (deamidation) | CDRL1 |
| | DP (chemical hydrolysis) | CDRL3 |
| 4A4.A3 | none | |

In addition, a putative sequence liability at M34, which falls within CDRH1 of 12H10.G7 under Kabat, was also identified. Variants of these antibodies were designed to remove the putative sequence liability motifs.

### Example 5. Humanization and Affinity Maturation

Based on the data collected regarding kinetics and affinity for recombinant hFLT3 protein, binding to cell lines expressing human and cynomolgus monkey FLT3, binding to different AML and ALL cancer cells, binning profile, as well as not inhibiting human FLT3-ligand binding, four mouse hybridoma subclones, namely 12H10.G7, 11F4.B9, 4A4.A3 and 14A5.E8, were selected for humanization. Although 4A4.A3 and 14A5.E8 showed slightly lower affinities to hFLT3 than 12H10.G7 and 11F4.B9, these antibodies appeared to bind to a unique epitope (not cross-blocking with reference antibodies) and Domain 1 of FLT3, respectively, and therefore were further analyzed for exploring epitope diversity.

The 12H10.G7 antibody was humanized to create GB94 and GB102 as described supra, which shared the same VH and VL sequences. Back mutations were introduced in the framework regions to create variants GB87 to GB93 and GB95 to GB101.

The 11F4.B9 antibody was humanized to create 1153 and 1154 as described supra, which shared the same VH and VL sequences. Back mutations were introduced in the framework regions to create variants 1151 and 1152. The 1153 antibody was also subject to affinity maturation. Briefly, a library focused on CDRs of the 1553 FLT3 scFv was designed and displayed on the surface of yeast. FACS selection was performed twice by incubating the yeast with biotinylated human FLT3-His antigen. The FACS-enriched output samples were combined with additional CDR mutants to make a second library. Two rounds of additional FACS selection were carried out by titrating with biotinylated human FLT3-His from 100 nM to 1 nM. Sorting was performed at 10 nM, where a clear increase in signal was observed for the library compared to the parent. Sorted yeast clones were plated and screened.

### Example 6. Assessment of Antibody Binding to Cells Expressed Human Cancer Antigens

Isogenic cell lines ectopically expressing human and cynomolgus monkey FLT3 were used to assess cross-reactivity between human and cynomolgus monkey FLT3. Human cancer cell line RMA expressing hFLT3 or cFLT3 was used to assess tumor antigen binding of FLT3-binding antibodies. The human AML cell lines MOLM-13 and MV4-11 and the human ALL cell line REH were used to assess binding ability of the antibodies. In particular, MOLM-13 cells, which expressed FLT3-T227M, was used to assess the ability of the anti-FLT3 antibody to bind a mutant FLT3.

The 1158 mAb, a monoclonal antibody humanized from 12H10.G7 in the human IgG1 format, was diluted and incubated with the respective cells. The cells were then incubated with a fluorophore conjugated anti-human IgG secondary antibody and were analyzed by flow cytometry. The mean fluorescence intensity (MFI) values were normalized to secondary antibody only controls to obtain fold over background (FOB) values.

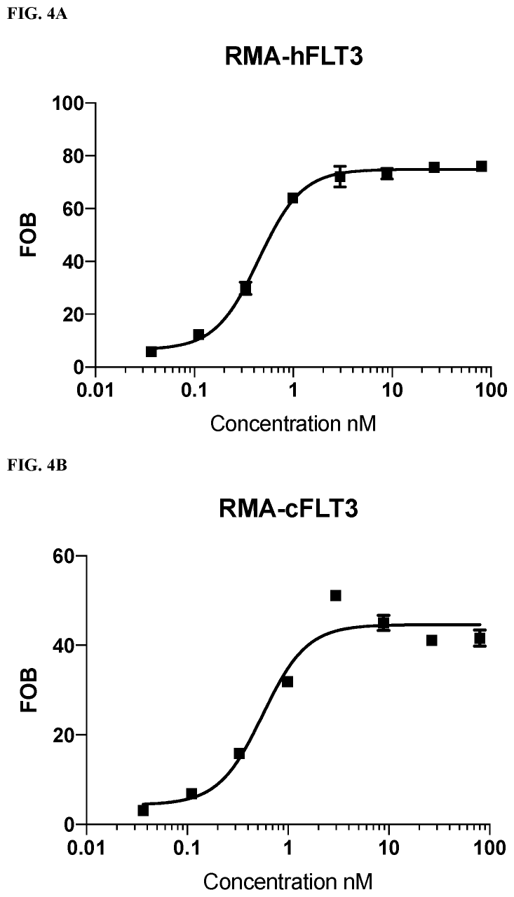

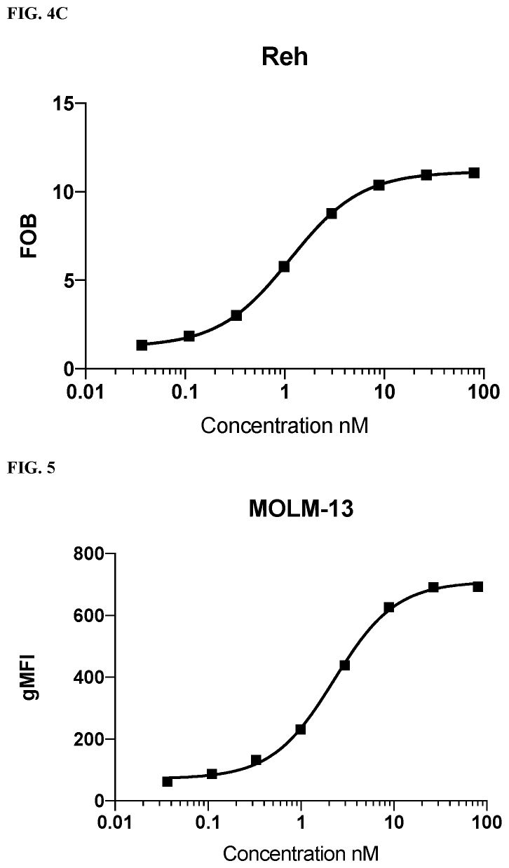

As shown in FIG. 4A and FIG. 4B, 1158 mAb bound RMA cells ectopically expressing human and cynomolgus FLT3 with equivalent potency. As shown in FIG. 4C, 1158 mAb bound REH cells, which were human ALL cells. As shown in FIG. 5, 1158 mAb bound MOLM-13 cells, which expressed FLT3-T227M.

MV4-11 cells, which expressed FLT3-ITD, was also used to assess the ability of the anti-FLT3 antibody to bind a mutant FLT3 using a similar method. It was observed that a bispecific antibody containing an antigen-binding site in the form of an scFv derived from the 1158 mAb bound MV4-11 cells.

### Example 7. Assessment of Antibody Internalization

The EOL-1 human cancer cell line, derived from eosinophilic leukaemia, was used to assess internalization of FLT3 after incubation with 1158 mAb. EOL-1 cells in duplicate plates were incubated with 1158 mAb or hIgG1 isotype control antibody at 37° C. for two hours. After incubation, the cells were washed and total FLT3 was stained using a non-competing anti-FLT3 antibody. Internalization of FLT3 was calculated as follows:

% internalization=(1−(sample MFI 2 hrs/hIgG1 isotype MFI 2 hrs))×100%

Internalization of FLT3 after incubation with 1158 mAb into REH cells, as measured using the method above, was about 8.27%. Internalization of FLT3 after incubation with 1158 mAb into EOL-1 cells, as measured using the method above, was about 8.30%.

### Example 8. Primary Human NK Cell Cytotoxicity Assay

Lysis of target cells was measured by the DELFIA cytotoxicity assay. Briefly, human cancer cell lines expressing FLT3 were harvested from culture, washed with HBS, and resuspended in growth media at 10⁶/mL for labeling with BATDA reagent (Perkin Elmer C136-100). Manufacturer instructions were followed for labeling of the target cells. After labeling, cells were washed three times with FIBS, and were resuspended at 0.5-1.0×10⁵/mL in culture media. 100 μl of BATDA labeled cells were added to each well of the 96-well plate. The 1158 mAb was diluted in culture media, and 50 μl of diluted mAb were added to each well.

To prepare NK cells, PBMCs were isolated from human peripheral blood buffy coats using density gradient centrifugation, washed, and prepared for NK cell isolation. NK cells were isolated using a negative selection technique with magnetic beads. Purity of isolated NK cells was typically >90% CD3-CD56+. Isolated NK cells were rested overnight and harvested from culture. The cells were then washed and resuspended at concentrations of 10⁵-2.0×10⁶/mL in culture media for an effector-to-target (E:T) ratio of 5:1. 50 μl of NK cells were added to each well of the plate for a total of 200 μl culture volume. The plate was incubated at 37° C. with 5% CO₂ for 2-3 hours.

After the incubation, the plate was removed from the incubator and the cells were pelleted by centrifugation at 200×g for 5 minutes. 20 μl of culture supernatant were transferred to a clean microplate and 200 μl of room temperature europium solution (Perkin Elmer C135-100) were added to each well. The plate was protected from light and incubated on a plate shaker at 250 rpm for 15 minutes, then read using SpectraMax i3X instruments.

Spontaneous release of substance that can form a fluorescent chelate with europium was measured in target cells incubated in the absence of NK cells. Maximum release of such substance was measured in target cells lysed with 1% Triton-X. % Specific lysis was calculated as follows:

% Specific lysis=((Experimental release−Spontaneous release)/(Maximum release−Spontaneous release))*100%.

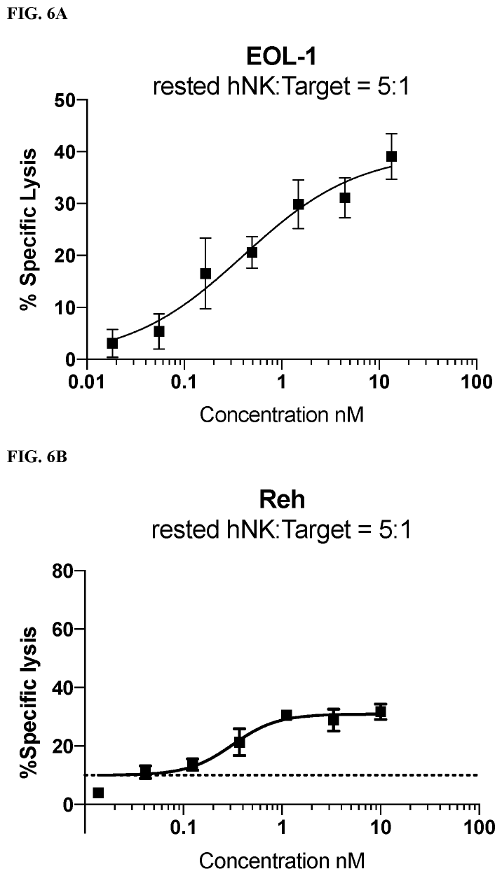

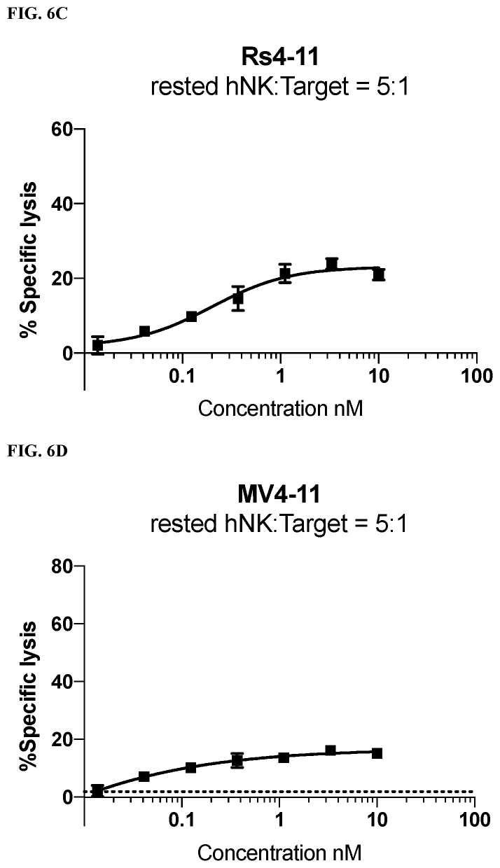

FIGS. 6A-6D show the activity of 1158 mAb in enhancing primary NK cell-mediated killing of human AML or ALL cell lines EOL-1 (FIG. 6A), Reh (FIG. 6B), RS4-11 (FIG. 6C), and MV4-11 (FIG. 6D). The 1158 mAb increased the ability of NK cells to kill target cells in a dose-dependent manner.

### Example 9. Assessment of TriNKET or mAb Binding to Whole Human Blood

The ability of 1158 mAb to bind different types of blood cells was assessed. Briefly, human whole blood was incubated with 1158 mAb or a human IgG1 isotype control antibody. The blood cells were analyzed by flow cytometry and binding of 1158 mAb or the isotype control antibody was detected using a fluorophore conjugated anti-human IgG secondary antibody.

No significant binding of 1158 mAb to granulocytes, monocytes, B cells, NK cells, CD8+ T cells, and CD4+ T cells in the blood was observed.

### Example 10. Activation of FLT3 Signaling

Phosphorylation of FLT3, a marker of FLT3 signaling, was measured by pFLT3 ELISA (R&D Systems DYC368). EOL-1 cells were plated in 96 well round bottom plates. The 1158 mAb and/or FLT3L were added. The samples were incubated at room temperature for 5 minutes and were immediately pelleted at 300×g for 5 minutes. The cells were washed twice with PBS. Cell pellets were resuspended in 200 μL of Lysis Buffer #9 and incubated on ice for 15 minutes. The samples were pelleted at 2000×g for 5 minutes, and the supernatants were transferred to clean test tubes. Protein concentrations were quantified using the BCA total protein assay. Samples were diluted in IC Diluent #12 as appropriate. Lysates were measured according to the manufacturer's instructions. pFLT3 concentration in each sample was determined by interpolation of values from the derived standard curve. Optical density values of the known standards were plotted against their respective concentrations and data was fit to a linear regression model.

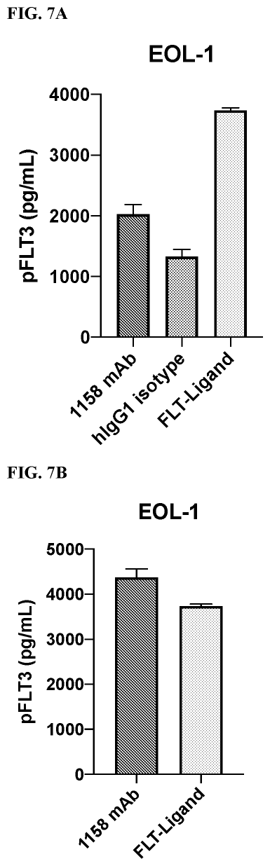

As shown in FIG. 7A, FLT3L led to a 3-fold increase in pFLT3 levels, whereas 1158 mAb did not induce significant FLT3 phosphorylation. FIG. 7B shows that when the cells were incubated with 1158 mAb in combination with FLT3L, 1158 mAb did not inhibit FLT3L-induced FLT3 phosphorylation. These results were consistent with the observation that 1158 mAb did not compete with FLT3L for binding FLT3.

### INCORPORATION BY REFERENCE

Unless stated to the contrary, the entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

SEQUENCE LISTING

| SEQ ID NO. | DESCRIPTION | SEQUENCE |
|---|---|---|
| 1 | 12H10.G7-VH | EVQLQESGPELVKPGASVKMSCKASGYTFTRYVMHWVKQRPGQGLEWI |
| | | GFINPYNDDTKYNEKFKGKATLTSDKSSSTAYMELSSLTSEDSAVYHC |
| | | ARWRQLGSLDSWGQGTTLTVSS |
| 2 | 12H10.G7-VL | NIVLTQSPASLAVSLGQRATISCRASESVDTYGSSFVHWYQQKPGQPP |
| | | KLLIYLASNLESGVPARFSGSGSRSDFTLTIDPVEADDAATYYCQQNN |
| | | EEPWTFGGGTKLEIK |
| 3 | ScFv of | QVQLVQSGAEVKKPGASVKVSCKASGYTFTRYVMHWVRQAPGQCLEWM |
| | humanized | GFINPYNDDTKYNEKFKGRVTITSDTSASTAYMELSSLRSEDTAVYHC |
| | 12H10.G7 | ARWRQLGSLDSWGQGTTVTVSSGGGGSGGGGSGGGGSGGGGSDIVMTQ |
| | GB87 (VH-VL) | SPASLAVSLGERATINCRASESVDTYGSSFVHWYQQKPGQPPKLLIYL |
| | | ASNLESGVPDRFSGSGSRTDFTLTISSLQAEDAATYYCQQNNEEPWTF |
| | | GCGTKVEIK |
| 4 | 12H10.G7-VH | NPYNDD |
| | CDR2 | |
| 5 | 12H10.G7-VH | WRQLGSLDS |
| | CDR3 | |
| 6 | 12H10.G7-VL | RASESVDTYGSSFVH |
| | CDR1 | |
| 7 | 12H10.G7-VL | LASNLES |
| | CDR2 | |
| 8 | 12H10.G7-VL | QQNNEEPWT |
| | CDR3 | |
| 9 | Humanized | QVQLVQSGAEVKKPGASVKVSCKASGYTFTRYVMHWVRQAPGQRLEWM |
| | 12H10.G7-VH | GFINPYNDDTKYNEKFKGRVTITSDTSASTAYMELSSLRSEDTAVYHC |
| | | ARWRQLGSLDSWGQGTTVTVSS |
| 10 | Humanized | DIVMTQSPASLAVSLGERATINCRASESVDTYGSSFVHWYQQKPGQPP |
| | 12H10.G7-VL | KLLIYLASNLESGVPDRFSGSGSRTDFTLTISSLQAEDAATYYCQQNN |
| | | EEPWTFGGGTKVEIK |
| 11 | Humanized | GYTFTRY |
| | 12H10.G7 | |
| | GB87/GB95-VH | |
| | CDR1 | |
| 12 | scFv of | DIVMTQSPASLAVSLGERATINCRASESVDTYGSSFVHWYQQKPGQPP |
| | humanized | KLLIYLASNLESGVPDRFSGSGSRTDFTLTISSLQAEDAATYYCQQNN |
| | 12H10.G7 | EEPWTFGCGTKVEIKGGGGSGGGGSGGGGSGGGGSQVQLVQSGAEVKK |
| | GB95 (VL-VH) | PGASVKVSCKASGYTFTRYVMHWVRQAPGQCLEWMGFINPYNDDTKYN |
| | | EKFKGRVTITSDTSASTAYMELSSLRSEDTAVYHCARWRQLGSLDSWG |
| | | QGTTVTVSS |
| 13 | Humanized | QVQLVQSGAEVKKPGASVKVSCKASGYTFTRYVMHWVRQAPGQRLEWM |
| | 12H10.G7 | GFINPYNDDTKYNEKFKGRVTITRDTSASTAYMELSSLRSEDTAVYHC |
| | GB88/GB96-VH | ARWRQLGSLDSWGQGTTVTVSS |
| 14 | Humanized | QVQLVQSGAEVKKPGASVKVSCKASGYSFTGYYIHWVRQGPGQGLEWM |
| | 11F4.B9 | GEIIPSTGSTIYAQKFQGRVTMTRDTSTSTVYMELSSLRSEDTAVYYC |
| | | ERWGDYYGRDYWGQGTLVTVSS |
| 15 | scFv of | QVQLVQSGAEVKKPGASVKVSCKASGYTFTRYVMHWVRQAPGQCLEWM |
| | humanized | GFINPYNDDTKYNEKFKGRVTITRDTSASTAYMELSSLRSEDTAVYHC |
| | 12H10.G7 | ARWRQLGSLDSWGQGTTVTVSSGGGGSGGGGSGGGGSGGGGSDIVMTQ |
| | GB88 (VH-VL) | SPASLAVSLGERATINCRASESVDTYGSSFVHWYQQKPGQPPKLLIYL |
| | | ASNLESGVPDRFSGSGSRTDFTLTISSLQAEDAATYYCQQNNEEPWTF |
| | | GCGTKVEIK |
| 16 | ScFv of | DIVMTQSPASLAVSLGERATINCRASESVDTYGSSFVHWYQQKPGQPP |
| | humanized | KLLIYLASNLESGVPDRFSGSGSRTDFTLTISSLQAEDAATYYCQQNN |
| | 12H10.G7 | EEPWTFGCGTKVEIKGGGGSGGGGSGGGGSGGGGSQVQLVQSGAEVKK |
| | GB96 (VL-VH) | PGASVKVSCKASGYTFTRYVMHWVRQAPGQCLEWMGFINPYNDDTKYN |
| | | EKFKGRVTITRDTSASTAYMELSSLRSEDTAVYHCARWRQLGSLDSWG |
| | | QGTTVTVSS |
| 17 | Humanized | QVQLVQSGAEVKKPGASVKVSCKASGYTFTRYVMHWVRQAPGQRLEWM |
| | 12H10.G7 | GFINPYNDDTKYNEKFKGRVTITSDTSASTAYMELSSLRSEDTAVYYC |
| | GB89/GB97-VH | ARWRQLGSLDSWGQGTTVTVSS |
| 18 | extracellular | NQDLPVIKCVLINHKNNDSSVGKSSSYPMVSESPEDLGCALRPQSSGT |
| | region of | VYEAAAVEVDVSASITLQVLVDAPGNISCLWVFKHSSLNCQPHFDLQN |
| | hFLT3-ITD | RGVVSMVILKMTETQAGEYLLFIQSEATNYTILFTVSIRNTLLYTLRR |
| | | PYFRKMENQDALVCISESVPEPIVEWVLCDSQGESCKEESPAVVKKEE |
| | | KVLHELFGTDIRCCARNELGRECTRLFTIDLNQTPQTTLPQLFLKVGE |
| | | PLWIRCKAVHVNHGFGLTWELENKALEEGNYFEMSTYSTNRTMIRILF |
| | | AFVSSVARNDTGYYTCSSSKHPSQSALVTIVEKGFINATNSSEDYEID |
| | | QYEEFCFSVRFKAYPQIRCTWTFSRKSFPCEQKGLDNGYSISKFCNHK |
| | | HQPGEYIFHAENDDAQFTKMFTLNIRRKPQVLAEASASQASCFSDGYP |
| | | LPSWTWKKCSDKSPNCTEEITEGVWNRKANRKVFGQWVSSSTLNMSEA |
| | | IKGFLVKCCAYNSLGTSCETILLNSPGPFPFIQDNIS |
| 19 | ScFv of | QVQLVQSGAEVKKPGASVKVSCKASGYTFTRYVMHWVRQAPGQCLEWM |
| | humanized | GFINPYNDDTKYNEKFKGRVTITSDTSASTAYMELSSLRSEDTAVYYC |
| | 12H10.G7 | ARWRQLGSLDSWGQGTTVTVSSGGGGSGGGGSGGGGSGGGGSDIVMTQ |
| | GB89 (VH-VL) | SPASLAVSLGERATINCRASESVDTYGSSFVHWYQQKPGQPPKLLIYL |
| | | ASNLESGVPDRFSGSGSRTDFTLTISSLQAEDAATYYCQQNNEEPWTF |
| | | GCGTKVEIK |
| 20 | scFv of | DIVMTQSPASLAVSLGERATINCRASESVDTYGSSFVHWYQQKPGQPP |
| | humanized | KLLIYLASNLESGVPDRFSGSGSRTDFTLTISSLQAEDAATYYCQQNN |
| | 12H10.G7 | EEPWTFGCGTKVEIKGGGGSGGGGSGGGGSGGGGSQVQLVQSGAEVKK |
| | GB97 (VL-VH) | PGASVKVSCKASGYTFTRYVMHWVRQAPGQCLEWMGFINPYNDDTKYN |
| | | EKFKGRVTITSDTSASTAYMELSSLRSEDTAVYYCARWRQLGSLDSWG |
| | | QGTTVTVSS |
| 21 | wild-type human | DKTHTCPPCPAPELLGGPSVFLFPPKPKDTLMISRTPEVTCVVVDVSH |
| | IgG1 Fc | EDPEVKFNWYVDGVEVHNAKTKPREEQYNSTYRVVSVLTVLHQDWLNG |
| | sequence | KEYKCKVSNKALPAPIEKTISKAKGQPREPQVYTLPPSRDELTKNQVS |
| | | LTCLVKGFYPSDIAVEWESNGQPENNYKTTPPVLDSDGSFFLYSKLTV |
| | | DKSRWQQGNVFSCSVMHEALHNHYTQKSLSLSPG |
| 22 | Humanized | DIVMTQSPDSLAVSLGERATINCRASESVDTYGSSFVHWYQQKPGQPP |
| | 12H10.G7 | KLLIYLASNLESGVPDRFSGSGSRTDFTLTISSLQAEDAATYYCQQNN |
| | GB90/GB98-VL | EEPWTFGGGTKVEIK |
| 23 | scFv of | QVQLVQSGAEVKKPGASVKVSCKASGYTFTRYVMHWVRQAPGQCLEWM |
| | humanized | GFINPYNDDTKYNEKFKGRVTITSDTSASTAYMELSSLRSEDTAVYHC |
| | 12H10.G7 | ARWRQLGSLDSWGQGTTVTVSSGGGGSGGGGSGGGGSGGGGSDIVMTQ |
| | GB90 (VH-VL) | SPDSLAVSLGERATINCRASESVDTYGSSFVHWYQQKPGQPPKLLIYL |
| | | ASNLESGVPDRFSGSGSRTDFTLTISSLQAEDAATYYCQQNNEEPWTF |
| | | GCGTKVEIK |
| 24 | scFv of | DIVMTQSPDSLAVSLGERATINCRASESVDTYGSSFVHWYQQKPGQPP |
| | humanized | KLLIYLASNLESGVPDRFSGSGSRTDFTLTISSLQAEDAATYYCQQNN |
| | 12H10.G7 | EEPWTFGCGTKVEIKGGGGSGGGGSGGGGSGGGGSQVQLVQSGAEVKK |
| | GB98 (VL-VH) | PGASVKVSCKASGYTFTRYVMHWVRQAPGQCLEWMGFINPYNDDTKYN |
| | | EKFKGRVTITSDTSASTAYMELSSLRSEDTAVYHCARWRQLGSLDSWG |
| | | QGTTVTVSS |
| 25 | extracellular | NQDLPVIKCVLINHKNNDSSVGKSSSYPMVSESPEDLGCALRPQSSGT |
| | region of | VYEAAAVEVDVSASITLQVLVDAPGNISCLWVFKHSSLNCQPHEDLQN |
| | hFLT3-T227M | RGVVSMVILKMTETQAGEYLLFIQSEATNYTILFTVSIRNTLLYTLRR |
| | | PYFRKMENQDALVCISESVPEPIVEWVLCDSQGESCKEESPAVVKKEE |
| | | KVLHELFGMDIRCCARNELGRECTRLFTIDLNQTPQTTLPQLFLKVGE |
| | | PLWIRCKAVHVNHGFGLTWELENKALEEGNYFEMSTYSTNRTMIRILF |
| | | AFVSSVARNDTGYYTCSSSKHPSQSALVTIVEKGFINATNSSEDYEID |
| | | QYEEFCFSVRFKAYPQIRCTWTFSRKSFPCEQKGLDNGYSISKFCNHK |
| | | HQPGEYIFHAENDDAQFTKMFTLNIRRKPQVLAEASASQASCFSDGYP |
| | | LPSWTWKKCSDKSPNCTEEITEGVWNRKANRKVFGQWVSSSTLNMSEA |
| | | IKGFLVKCCAYNSLGTSCETILLNSPGPFPFIQDNIS |
| 26 | scFv of | DIVMTQSPASLAVSLGERATINCRASESVDTYGSSFVHWYQQKPGQPP |
| | humanized | KLLIYLASNLESGVPDRFSGSGSGTDFTLTISSLQAEDAATYYCQQNN |
| | 12H10.G7-VL | EEPWTFGGGTKVEIK |
| 27 | scFv of | QVQLVQSGAEVKKPGASVKVSCKASGYTFTRYVMHWVRQAPGQCLEWM |
| | humanized | GFINPYNDDTKYNEKFKGRVTITSDTSASTAYMELSSLRSEDTAVYHC |
| | 12H10.G7 | ARWRQLGSLDSWGQGTTVTVSSGGGGSGGGGSGGGGSGGGGSDIVMTQ |
| | GB91 (VH-VL) | SPASLAVSLGERATINCRASESVDTYGSSFVHWYQQKPGQPPKLLIYL |
| | | ASNLESGVPDRFSGSGSGTDFTLTISSLQAEDAATYYCQQNNEEPWTF |
| | | GCGTKVEIK |
| 28 | scFv of | DIVMTQSPASLAVSLGERATINCRASESVDTYGSSFVHWYQQKPGQPP |
| | humanized | KLLIYLASNLESGVPDRFSGSGSGTDFTLTISSLQAEDAATYYCQQNN |
| | 12H10.G7 | EEPWTFGCGTKVEIKGGGGSGGGGSGGGGSGGGGSQVQLVQSGAEVKK |
| | GB99 (VL-VH) | PGASVKVSCKASGYTFTRYVMHWVRQAPGQCLEWMGFINPYNDDTKYN |
| | | EKFKGRVTITSDTSASTAYMELSSLRSEDTAVYHCARWRQLGSLDSWG |
| | | QGTTVTVSS |
| 29 | Humanized | QVQLVQSGAEVKKPGASVKVSCKVSGYTFX₁X₂YWINWVRQX₃PGKX₄LE |
| | 14A5.E8 | WMGNIYPGSSIINYNENFKNRVTMTX₅DTSX₆DTAYMELSSLRSEDTAV |
| | consensus-VH | YYCARRX₇VYLX₈FDYWGQGTLVTVSS, where X₁ is P or T, |
| | | X₂ is S or Y, X₃ is A or R, X4 is C or G, X₅ is V |
| | | or E, X₆ is S or T, X₇ is N or V, and X₈ is T or Y |
| 30 | Humanized | DIVMTQSPASLAVSLGERATINCRASESVDTYGSSFVHWYQQKPGQPP |
| | 12H10.G7 | KLLIYLASNLESGVPDRFSGSGSRTDFTLTISSLQAEDVATYYCQQNN |
| | GB92/GB100-VL | EEPWTFGGGTKVEIK |
| 31 | scFv of | QVQLVQSGAEVKKPGASVKVSCKASGYTFTRYVMHWVRQAPGQCLEWM |
| | humanized | GFINPYNDDTKYNEKFKGRVTITSDTSASTAYMELSSLRSEDTAVYHC |
| | 12H10.G7 | ARWRQLGSLDSWGQGTTVTVSSGGGGSGGGGSGGGGSGGGGSDIVMTQ |
| | GB92 (VH-VL) | SPASLAVSLGERATINCRASESVDTYGSSFVHWYQQKPGQPPKLLIYL |
| | | ASNLESGVPDRFSGSGSRTDFTLTISSLQAEDVATYYCQQNNEEPWTF |
| | | GCGTKVEIK |
| 32 | ScFv of | DIVMTQSPASLAVSLGERATINCRASESVDTYGSSFVHWYQQKPGQPP |
| | humanized | KLLIYLASNLESGVPDRFSGSGSRTDFTLTISSLQAEDVATYYCQQNN |
| | 12H10.G7 | EEPWTFGCGTKVEIKGGGGSGGGGSGGGGSGGGGSQVQLVQSGAEVKK |
| | GB100 (VL-VH) | PGASVKVSCKASGYTFTRYVMHWVRQAPGQCLEWMGFINPYNDDTKYN |
| | | EKFKGRVTITSDTSASTAYMELSSLRSEDTAVYHCARWRQLGSLDSWG |
| | | QGTTVTV |
| 33 | 4H2.E3-VH CDR2 | NPYSDG |
| 34 | Humanized | DIVMTQSPASLAVSLGERATINCRASESVDTYGSSFVHWYQQKPGQPP |
| | 12H10.G7 | KLLIYLASNLESGVPDRFSGSGSRTDFTLTISSLQAEDAAVYYCQQNN |
| | GB93/GB101-VL | EEPWTFGGGTKVEIK |
| 35 | scFv of | QVQLVQSGAEVKKPGASVKVSCKASGYTFTRYVMHWVRQAPGQCLEWM |
| | humanized | GFINPYNDDTKYNEKFKGRVTITSDTSASTAYMELSSLRSEDTAVYHC |
| | 12H10.G7 | ARWRQLGSLDSWGQGTTVTVSSGGGGSGGGGSGGGGSGGGGSDIVMTQ |
| | GB93 (VH-VL) | SPASLAVSLGERATINCRASESVDTYGSSFVHWYQQKPGQPPKLLIYL |
| | | ASNLESGVPDRFSGSGSRTDFTLTISSLQAEDAAVYYCQQNNEEPWTF |
| | | GCGTKVEIK |
| 36 | scFv of | DIVMTQSPASLAVSLGERATINCRASESVDTYGSSFVHWYQQKPGQPP |
| | humanized | KLLIYLASNLESGVPDRFSGSGSRTDFTLTISSLQAEDAAVYYCQQNN |
| | 12H10.G7 | EEPWTFGCGTKVEIKGGGGSGGGGSGGGGSGGGGSQVQLVQSGAEVKK |
| | GB101 (VL-VH) | PGASVKVSCKASGYTFTRYVMHWVRQAPGQCLEWMGFINPYNDDTKYN |
| | | EKFKGRVTITSDTSASTAYMELSSLRSEDTAVYHCARWRQLGSLDSWG |
| | | QGTTVTVSS |
| 37 | Humanized | QVQLVQSGAEVKKPGASVKVSCKASGYTFTRYVMHWVRQAPGQRLEWM |
| | 12H10.G7 | GFINPYNDDTKYNEKFKGRVTITRDTSASTAYMELSSLRSEDTAVYYC |
| | GB94/GB102-VH | ARWRQLGSLDSWGQGTTVTVSS |
| 38 | Humanized | DIVMTQSPDSLAVSLGERATINCRASESVDTYGSSFVHWYQQKPGQPP |
| | 12H10.G7 | KLLIYLASNLESGVPDRFSGSGSGTDFTLTISSLQAEDVAVYYCQQNN |
| | GB94/GB102-VL | EEPWTFGGGTKVEIK |
| 39 | scFv of | QVQLVQSGAEVKKPGASVKVSCKASGYTFTRYVMHWVRQAPGQCLEWM |
| | humanized | GFINPYNDDTKYNEKFKGRVTITRDTSASTAYMELSSLRSEDTAVYYC |
| | 12H10.G7 | ARWRQLGSLDSWGQGTTVTVSSGGGGSGGGGSGGGGSGGGGSDIVMTQ |
| | GB94 (VH-VL) | SPDSLAVSLGERATINCRASESVDTYGSSFVHWYQQKPGQPPKLLIYL |
| | | ASNLESGVPDRFSGSGSGTDFTLTISSLQAEDVAVYYCQQNNEEPWTF |
| | | GCGTKVEIK |
| 40 | scFv of | DIVMTQSPDSLAVSLGERATINCRASESVDTYGSSFVHWYQQKPGQPP |
| | humanized | KLLIYLASNLESGVPDRFSGSGSGTDFTLTISSLQAEDVAVYYCQQNN |
| | 12H10.G7 | EEPWTFGCGTKVEIKGGGGSGGGGSGGGGSGGGGSQVQLVQSGAEVKK |
| | GB102 (VL-VH) | PGASVKVSCKASGYTFTRYVMHWVRQAPGQCLEWMGFINPYNDDTKYN |
| | | EKFKGRVTITRDTSASTAYMELSSLRSEDTAVYYCARWRQLGSLDSWG |
| | | QGTTVTVSS |
| 41 | Humanized | QVQLVQSGAEVKKPGASVKVSCKASGYTFTRYVMHWVRQAPGQRLEWM |
| | 12H10.G7 GB102 | GFINPYNDDTKYNEKFKGRVTITRDTSASTAYMELSSLRSEDTAVYYC |
| | D101E-VH | ARWRQLGSLESWGQGTTVTVSS |
| 42 | Humanized | DIVMTQSPDSLAVSLGERATINCRASESVDTYGSSFVHWYQQKPGQPP |
| | 12H10.G7 GB102 | KLLIYLASNLESGVPDRFSGSGSGTDFTLTISSLQAEDVAVYYCQQNN |
| | -VL | EEPWTFGCGTKVEIK |
| 43 | SCFv of | QVQLVQSGAEVKKPGASVKVSCKASGYTFTRYVMHWVRQAPGQCLEWM |
| | humanized | GFINPYNDDTKYNEKFKGRVTITRDTSASTAYMELSSLRSEDTAVYYC |
| | 12H10.G7 GB102 | ARWRQLGSLESWGQGTTVTVSSGGGGSGGGGSGGGGSGGGGSDIVMTQ |
| | D101E (VH-VL) | SPDSLAVSLGERATINCRASESVDTYGSSFVHWYQQKPGQPPKLLIYL |
| | | ASNLESGVPDRFSGSGSGTDFTLTISSLQAEDVAVYYCQQNNEEPWTF |
| | | GCGTKVEIK |
| 44 | scFv of | DIVMTQSPDSLAVSLGERATINCRASESVDTYGSSFVHWYQQKPGQPP |
| | humanized | KLLIYLASNLESGVPDRFSGSGSGTDFTLTISSLQAEDVAVYYCQQNN |
| | 12H10.G7 GB102 | EEPWTFGCGTKVEIKGGGGSGGGGSGGGGSGGGGSQVQLVQSGAEVKK |
| | D101E (VL-VH) | PGASVKVSCKASGYTFTRYVMHWVRQAPGQCLEWMGFINPYNDDTKYN |
| | | EKFKGRVTITRDTSASTAYMELSSLRSEDTAVYYCARWRQLGSLESWG |
| | | QGTTVTVSS |
| 45 | Humanized | QVQLVQSGAEVKKPGASVKVSCKASGYTFTRYVIHWVRQAPGQRLEWM |
| | 12H10.G7 GB102 | GFINPYNDDTKYNEKFKGRVTITRDTSASTAYMELSSLRSEDTAVYYC |
| | M34I-VH | ARWRQLGSLDSWGQGTTVTVSS |
| 46 | 14H8.E7-VLCDR3 | QQWSSKSPT |
| 47 | scFv of | QVQLVQSGAEVKKPGASVKVSCKASGYTFTRYVIHWVRQAPGQCLEWM |
| | humanized | GFINPYNDDTKYNEKFKGRVTITRDTSASTAYMELSSLRSEDTAVYYC |
| | 12H10.G7 GB102 | ARWRQLGSLDSWGQGTTVTVSSGGGGSGGGGSGGGGSGGGGSDIVMTQ |
| | M34I(VH-VL) | SPDSLAVSLGERATINCRASESVDTYGSSFVHWYQQKPGQPPKLLIYL |
| | | ASNLESGVPDRFSGSGSGTDFTLTISSLQAEDVAVYYCQQNNEEPWTF |
| | | GCGTKVEIK |
| 48 | scFv of | DIVMTQSPDSLAVSLGERATINCRASESVDTYGSSFVHWYQQKPGQPP |
| | humanized | KLLIYLASNLESGVPDRFSGSGSGTDFTLTISSLQAEDVAVYYCQQNN |
| | 12H10.G7 GB102 | EEPWTFGCGTKVEIKGGGGSGGGGSGGGGSGGGGSQVQLVQSGAEVKK |
| | M34I(VL-VH) | PGASVKVSCKASGYTFTRYVIHWVRQAPGQCLEWMGFINPYNDDTKYN |
| | | EKFKGRVTITRDTSASTAYMELSSLRSEDTAVYYCARWRQLGSLDSWG |
| | | QGTTVTVSS |
| 49 | Humanized | QVQLVQSGAEVKKPGASVKVSCKASGYTFTRYVIHWVRQAPGQRLEWM |
| | 12H10.G7 GB102 | GFINPYNDDTKYNEKFKGRVTITRDTSASTAYMELSSLRSEDTAVYYC |
| | M34I/D101E-VH | ARWRQLGSLESWGQGTTVTVSS |
| 50 | Humanized | WRQLGSLES |
| | 12H10.G7 GB102 | |
| | M34I/D101E - | |
| | CDR3 | |
| 51 | ScFv of | QVQLVQSGAEVKKPGASVKVSCKASGYTFTRYVIHWVRQAPGQCLEWM |
| | humanized | GFINPYNDDTKYNEKFKGRVTITRDTSASTAYMELSSLRSEDTAVYYC |
| | 12H10.G7 GB102 | ARWRQLGSLESWGQGTTVTVSSGGGGSGGGGSGGGGSGGGGSDIVMTQ |
| | M34I/D101E | SPDSLAVSLGERATINCRASESVDTYGSSFVHWYQQKPGQPPKLLIYL |
| | (VH-VL) | ASNLESGVPDRFSGSGSGTDFTLTISSLQAEDVAVYYCQQNNEEPWTF |
| | | GCGTKVEIK |
| 52 | scFv of | DIVMTQSPDSLAVSLGERATINCRASESVDTYGSSFVHWYQQKPGQPP |
| | humanized | KLLIYLASNLESGVPDRFSGSGSGTDFTLTISSLQAEDVAVYYCQQNN |
| | 12H10.G7 GB102 | EEPWTFGCGTKVEIKGGGGSGGGGSGGGGSGGGGSQVQLVQSGAEVKK |
| | M34I/D101E | PGASVKVSCKASGYTFTRYVIHWVRQAPGQCLEWMGFINPYNDDTKYN |
| | (VL-VH) | EKFKGRVTITRDTSASTAYMELSSLRSEDTAVYYCARWRQLGSLESWG |
| | | QGTTVTVSS |
| 53 | Humanized | QVQLVQSGAEVKKPGASVKVSCKASGYTFTRYVX₁HWVRQAPGQRLEW |
| | 12H10.G7 | MGFINPYNDDTKYNEKFKGRVTITRDTSASTAYMELSSLRSEDTAVYY |
| | consensus 1-VH | CARWRQLGSLX₂SWGQGTTVTVSS, where X₁ is M or I, and |
| | | X₂ is E or D |
| 54 | Humanized | RX₁VYLX₂FDY, where X₁ is N or V, and X₂ is T or Y |
| | 14A5.E8 | |
| | consensus VH | |
| | CDR3 | |
| 55 | Humanized | WRQLGSLXS, where X is E or D |
| | 12H10.G7 | |
| | consensus 1-VH | |
| | CDR3 | |
| 56 | Humanized | QVQLVQSGAEVKKPGASVKVSCKASGYTFTRYVMHWVRQAPGQRLEWM |
| | 12H10.G7 | GFINPYNDDTKYNEKFKGRVTITX₁DTSASTAYMELSSLRSEDTAVYX₂ |
| | consensus 2 -VH | CARWRQLGSLDSWGQGTTVTVSS, where X₁ is Sor R, and |
| | | X₂ is Y or H |
| 57 | Humanized | DIVMTQSPX₁SLAVSLGERATINCRASESVDTYGSSFVHWYQQKPGQP |
| | 12H10.G7 | PKLLIYLASNLESGVPDRFSGSGSX₂TDFTLTISSLQAEDX₃AX₄YYCQ |
| | consensus 2 -VL | QNNEEPWTFGGGTKVEIK, where X₁ is A or D, X₂ is R |
| | | or G, and X₃ is A or V, and X₄ is T or V |
| 58 | Humanized | QVQLVQSGAEVKKPGASVKVSCKASGYTFTRYVX₁HWVRQAPGQCLEW |
| | 12H10.G7 | MGFINPYNDDTKYNEKFKGRVTITRDTSASTAYMELSSLRSEDTAVYY |
| | consensus 3-VH | CARWRQLGSLX₂SWGQGTTVTVSS, where X₁ is M or I, and |
| | | X₂ is E or D |
| 59 | Humanized | GYTFX₁X₂Y, where X₁ is P or T, and X₂ is Sor Y |
| | 14A5.E8 | |
| | consensus VH | |
| | CDR1 | |
| 60 | 14A5.E8-VH | EVQLQESGAELVQPGASVRLSCKASGYTFTSYWINWVKQRPGQGLEWI |
| | | GNIYPGSSIINYNENFKNRATLTVDTSSSTAYMQLSSLTSDDSAVYYC |
| | | ARRVVYLYFDYWGQGTTLTVSS |
| 61 | 14A5.E8-VL | QIVLTQSPAIMSASPGEKVTMTCSASSSVSYMHWYQQKSGTSPKRWIY |
| | | DTSKLASGVPARFSGSGSGTSYSLTISSMEAEDAATYYCQQWTSKSPT |
| | | FGGGTKLEIK |
| 62 | 14A5.E8-VH CDR1 | GYTFTSY |
| 63 | 14A5. E8-VH or | YPGSSI |
| | Humanized | |
| | 14A5.E8 | |
| | consensus VH | |
| | CDR2 | |
| 64 | 14A5.E8-VH CDR3 | RVVYLYFDY |
| 65 | 14A5.E8-VLCDR1 | SASSSVSYMH |
| 66 | 14A5. E8-VLor | DTSKLAS |
| | Humanized | |
| | 14A5.E8 | |
| | consensus VL | |
| | CDR2 | |
| 67 | 14A5. E8-VLor | QQWTSKSPT |
| | Humanized | |
| | 14A5.E8 | |
| | consensus VL | |
| | CDR3 | |
| 68 | Humanized | QVQLVQSGAEVKKPGASVKVSCKVSGYTFTSYWINWVRQRPGKGLEWM |
| | 14A5.E8 | GNIYPGSSIINYNENFKNRVTMTVDTSSDTAYMELSSLRSEDTAVYYC |
| | 1551/1552-VH | ARRVVYLYFDYWGQGTLVTVSS |
| 69 | Humanized | EIVLTQSPATLSLSPGEKATLSCSASSSVSYMHWYQQKPGQAPRLLIY |
| | 14A5.E8 | DTSKLASGIPARFSGSGSGTSFTLTISSLEPEDAAVYYCQQWTSKSPT |
| | 1551/1552-VL | FGGGTKVEIK |
| 70 | scFv of | QVQLVQSGAEVKKPGASVKVSCKVSGYTFTSYWINWVRQRPGKCLEWM |
| | humanized | GNIYPGSSIINYNENFKNRVTMTVDTSSDTAYMELSSLRSEDTAVYYC |
| | 14A5.E8 | ARRVVYLYFDYWGQGTLVTVSSGGGGSGGGGSGGGGSGGGGSEIVLTQ |
| | 1551 (VH-VL) | SPATLSLSPGEKATLSCSASSSVSYMHWYQQKPGQAPRLLIYDTSKLA |
| | | SGIPARFSGSGSGTSFTLTISSLEPEDAAVYYCQQWTSKSPTFGCGTK |
| | | VEIK |
| 71 | ScFv of | EIVLTQSPATLSLSPGEKATLSCSASSSVSYMHWYQQKPGQAPRLLIY |
| | humanized | DTSKLASGIPARFSGSGSGTSFTLTISSLEPEDAAVYYCQQWTSKSPT |
| | 14A5.E8 | FGCGTKVEIKGGGGSGGGGSGGGGSGGGGSQVQLVQSGAEVKKPGASV |
| | 1552 (VL-VH) | KVSCKVSGYTFTSYWINWVRQRPGKCLEWMGNIYPGSSIINYNENFKN |
| | | RVTMTVDTSSDTAYMELSSLRSEDTAVYYCARRVVYLYFDYWGQGTLV |
| | | TVSS |
| 72 | Humanized | QVQLVQSGAEVKKPGASVKVSCKVSGYTFTSYWINWVRQAPGKGLEWM |
| | 14A5.E8 | GNIYPGSSIINYNENFKNRVTMTEDTSTDTAYMELSSLRSEDTAVYYC |
| | 1553/1554-VH | ARRVVYLYFDYWGQGTLVTVSS |
| 73 | Humanized | EIVLTQSPATLSLSPGERATLSCSASSSVSYMHWYQQKPGQAPRLLIY |
| | 14A5.E8 | DTSKLASGIPARFSGSGSGTDFTLTISSLEPEDFAVYYCQQWTSKSPT |
| | 1553/1554-VL | FGGGTKVEIK |
| 74 | ScFv of | QVQLVQSGAEVKKPGASVKVSCKVSGYTFTSYWINWVRQAPGKCLEWM |
| | humanized | GNIYPGSSIINYNENFKNRVTMTEDTSTDTAYMELSSLRSEDTAVYYC |
| | 14A5.E8 | ARRVVYLYFDYWGQGTLVTVSSGGGGSGGGGSGGGGSGGGGSEIVLTQ |
| | 1553 (VH-VL) | SPATLSLSPGERATLSCSASSSVSYMHWYQQKPGQAPRLLIYDTSKLA |
| | | SGIPARFSGSGSGTDFTLTISSLEPEDFAVYYCQQWTSKSPTFGCGTK |
| | | VEIK |
| 75 | scFv of | EIVLTQSPATLSLSPGERATLSCSASSSVSYMHWYQQKPGQAPRLLIY |
| | humanized | DTSKLASGIPARFSGSGSGTDFTLTISSLEPEDFAVYYCQQWTSKSPT |
| | 14A5.E8 | FGCGTKVEIKGGGGSGGGGSGGGGSGGGGSQVQLVQSGAEVKKPGASV |
| | 1554 (VL-VH) | KVSCKVSGYTFTSYWINWVRQAPGKCLEWMGNIYPGSSIINYNENFKN |
| | | RVTMTEDTSTDTAYMELSSLRSEDTAVYYCARRVVYLYFDYWGQGTLV |
| | | TVSS |
| 76 | Humanized | QVQLVQSGAEVKKPGASVKVSCKVSGYTFPYYWINWVRQAPGKGLEWM |
| | 14A5.E8 1689-VH | GNIYPGSSIINYNENFKNRVTMTEDTSTDTAYMELSSLRSEDTAVYYC |
| | | ARRNVYLTFDYWGQGTLVTVSS |
| 77 | Humanized | EIVLTQSPATLSLSPGERATLSCSASSSVSYIHWYQQKPGQAPRLLIY |
| | 14A5.E8 1689-VL | DTSKLASGIPARFSGSGSGTDFTLTISSLEPEDFAVYYCQQWTSKSPT |
| | | FGGGTKVEIK |
| 78 | Humanized | GYTFPYY |
| | 14A5.E8 1689-VH | |
| | CDR1 | |
| 79 | Humanized | RNVYLTFDY |
| | 14A5.E8 1689-VH | |
| | CDR3 | |
| 80 | Humanized | SASSSVSYIH |
| | 14A5.E8 1689-VL | |
| | CDR1 | |
| 81 | scFv of | QVQLVQSGAEVKKPGASVKVSCKVSGYTFPYYWINWVRQAPGKCLEWM |
| | humanized | GNIYPGSSIINYNENFKNRVTMTEDTSTDTAYMELSSLRSEDTAVYYC |
| | 14A5.E8 1689 | ARRNVYLTFDYWGQGTLVTVSSGGGGSGGGGSGGGGSGGGGS |
| | (VH-VL) | EIVLTQSPATLSLSPGERATLSCSASSSVSYIHWYQQKPGQAPRLLIY |
| | | DTSKLASGIPARFSGSGSGTDFTLTISSLEPEDFAVYYCQQWTSKSPT |
| | | FGCGTKVEIK |
| 82 | scFv of | EIVLTQSPATLSLSPGERATLSCSASSSVSYIHWYQQKPGQAPRLLIY |
| | humanized | DTSKLASGIPARFSGSGSGTDFTLTISSLEPEDFAVYYCQQWTSKSPT |
| | 14A5.E8 1689 | FGCGTKVEIKGGGGSGGGGSGGGGSGGGGSQVQLVQSGAEVKKPGASV |
| | (VL-VH) | KVSCKVSGYTFPYYWINWVRQAPGKCLEWMGNIYPGSSIINYNENFKN |
| | | RVTMTEDTSTDTAYMELSSLRSEDTAVYYCARRNVYLTFDYWGQGTLV |
| | | TVSS |
| 83 | 14H8.E7-VL | QIVLTQSPAIMSASPGEKVTMTCSASSSVSYMHWYQQKSGTSPKRWIF |
| | | DTSKLASGVPVRESGSGSGTSYSLTITNMETEDAATYYCQQWSSKSPT |
| | | FGGGTKLEIK |
| 84 | Humanized | EIVLTQSPATLSLSPGERATLSCSASSSVSYXHWYQQKPGQAPRLLIY |
| | 14A5.E8 | DTSKLASGIPARFSGSGSGTDFTLTISSLEPEDFAVYYCQQWTSKSPT |
| | consensus VL | FGGGTKVEIK, where X is M or I |
| 85 | 11F4.B9-VH | EVQLQESGPELVKPGASVKISCKASGYSFTGYYIHWVKQGPEKSLEWI |
| | | GEIIPSTGSTIYNQKFKAKATLTVDKSSSTAYLQLKSLTSEDSAVYYC |
| | | ERWGDYYGRDYWGQGTSVTVSS |
| 86 | Humanized | SASSSVSYXH, where X is M or I |
| | 14A5.E8 | |
| | consensus VL | |
| | CDR1 | |
| 87 | 11F4.B9-VH CDR1 | GYSFTGY |
| 88 | 11F4.B9-VH CDR2 | IPSTGS |
| 89 | 11F4.B9-VH CDR3 | WGDYYGRDY |
| 90 | 11F4.B9-VL | DIVLTQSPASLAVSLGQRATISCRASESVDIYGNSFMHWYQQKPGQPP |
| | | KLLIYRASNLESGIPARFSGSGSRTDFTLTINPVEADDVATYYCQQSN |
| | | EDPRTFGGGTKLEIK |
| 91 | 11F4.B9-VLCDR1 | RASESVDIYGNSFMH |
| 92 | 11F4.B9-VLCDR2 | RASNLES |
| 93 | 11F4.B9-VLCDR3 | QQSNEDPRT |
| 94 | Humanized | DIVMTQSPASLAVSLGERATINCRASESVDIYGNSFMHWYQQKPGQPP |
| | 11F4.B9-VL | KLLIYRASNLESGVPDRFSGSGSRTDFTLTINSLQAEDVATYYCQQSN |
| | | EDPRTFGGGTKVEIK |
| 95 | 4A4.A3-VH | QVTLKESGPGILQPSQTLSLTCSFSGFSLTTYGMGVGWIRQPSGKGLE |
| | | WLANIWFNDNKYYNSTLKSRLTISKDTSNNQVFLKISSVDTTDTATYY |
| | | CAQITTVVGTEDYWGQGSPLTVSP |
| 96 | 4A4.A3-VL | RIVMTQSPTTMAASPGEKITITCSASSSISSIYLHWYQQKPGFSPKLL |
| | | IFRTSDLASGVPPRFGGSGSGTSYSLTIGTMEAEDVATYYCQQGSSFP |
| | | RTFGGGTKLEIK |
| 97 | 4A4.A3-VH CDR1 | GFSLTTYGM |
| 98 | 4A4.H7-VH CDR2 | YPNTGI |
| 99 | 4A4.A3-VH CDR2 | WENDN |
| 100 | 4A4.A3-VH CDR3 | ITTVVGTEDY |
| 101 | 4A4.A3-VLCDR1 | SASSSISSIYLH |
| 102 | 4A4.A3-VLCDR2 | RTSDLAS |
| 103 | 4A4.A3-VLCDR3 | QQGSSFPRT |
| 104 | 4A4.H7-VH | EVQLQESGPELVKPGASVKISCKASGYSFTGYYIHWVKQSPEESLEWI |
| | | GEIYPNTGITTYNQKFTAKATLTVDKSSNTAYMQLKSLTSEDSAVYYC |
| | | TRWGDYYGRDYWGQGTSVTVSS |
| 105 | 4A4.H7-VL | DIVLTQSPASLAVSLGQRATISCRASETVDTHGNSFMHWYQQKPGQPP |
| | | KLLIYRASNLESGIPARFSGSGSRTDFTLTINPVEADDVATYYCQQSN |
| | | EDPRTFGGGTKLEIK |
| 106 | 4A4.H7-VLCDR1 | RASETVDTHGNSFMH |
| 107 | 15A11.C8-VH | EVQLQESGGGLVKTGGSRKLSCAASGFTFSDYGMHWVRHTPEKGLEWV |
| | | VYISSGGNTIFYTDTVKGRFTISRDNAKNTLFLQMTSLRSEDTAVYFC |
| | | VRQGYYYAMDYWGQGASVTVSS |
| 108 | 15A11.C8-VL | DIQMTQTTSSLSASLGDRVTIRCRASQDITNYLNWYQQKPDGAVKLLI |
| | | SYTSILQSGVPSRFSGSGSGTDYSLTISNLEQGDVATYFCQQGSSLPW |
| | | TFGGGTKLEIK |
| 109 | 15A11.C8-VH | GFTFSDY |
| | CDR1 | |
| 110 | 15A11.C8-VH | SSGGNT |
| | CDR2 | |
| 111 | 15A11.C8-VH | QGYYYAMDY |
| | CDR3 | |
| 112 | 15A11.C8-VL | RASQDITNYLN |
| | CDR1 | |
| 113 | 15A11.C8-VL | YTSILQS |
| | CDR2 | |
| 114 | 15A11.C8-VL | QQGSSLPWT |
| | CDR3 | |
| 115 | 12C9.E5-VH | EVQLQESGAELVRPGASVKLSCKASGYIFTDYEIHWVKQTPVHGLEWI |
| | | GAIDPETGITAYSQKFKGKATLTTDTSSSTAYMEFRSLTSEDSAVYYC |
| | | TRGGLLYWGQGTSVTVSS |
| 116 | 12C9.E5-VL | DVVMTQTPLSLSVTIGQPASISCKSSQSLLYSDGETYLNWLQQRPGQS |
| | | PKRLMYQVSKLDPGIPDRFSGSGSETDFTLKISRVEAEDLGIYYCLQG |
| | | TFYPHTFGGGTKLEIK |
| 117 | 12C9. E5-VH CDR1 | GYIFTDY |
| 118 | 12C9.E5-VH CDR2 | DPETGI |
| 119 | 12C9.E5-VH CDR3 | GGLLY |
| 120 | 12C9.E5-VLCDR1 | KSSQSLLYSDGETYLN |
| 121 | 12C9.E5-VLCDR2 | QVSKLDP |
| 122 | 12C9.E5-VLCDR3 | LQGTFYPHT |
| 123 | 1A2.A3 VH | EVQLQESGPELVKPGASVKISCKASGYSFTGYYIHWVKQSPEESLEWI |
| | | GEIYPNTGITTYNQKFTAKATLTVDKSSNTAYMQLKSLTSEDSAVYYC |
| | | TRWGDYYGRDYWGQGTSVTVSS |
| 124 | 1A2.A3-VL | DIVLTQSPASLAVSLGQRATISCRASETVDTHGNSFMHWYQQKPGQPP |
| | | KLLIYRASNLESGIPARFSGSGSRTDFTLTINPVEADDVATYYCQQSN |
| | | EDPRTFGGGTKLEIK |
| 125 | 4H2.E3-VH | EVQLQESGPELVKPGASVKMSCKASGYTFTSYLMHWMKQKPGQGLEWI |
| | | GYINPYSDGIKYNEKFRDKATLTSDKSSNTAYMELSSLTSEDSAVYYC |
| | | AHSSGYVGYAMDYWGQGTSVTVSS |
| 126 | 4H2.E3-VL | GIVMTQTTPSVPVTPGESVSISCRSSKSLLHSNGNTYLYWFLQRPGQS |
| | | PQLLIYRMSNLASGVPDRFSGSGSGTTFTLRISRVEAEDVGVYYCMQH |
| | | LEYPFTFGSGTKLEIK |
| 127 | 4H2.E3-VH CDR3 | SSGYVGYAMDY |
| 128 | 4H2.E3-VLCDR1 | RSSKSLLHSNGNTYLY |
| 129 | 4H2.E3-VLCDR2 | RMSNLAS |
| 130 | 4H2.E3-VLCDR3 | MQHLEYPFT |
| 131 | 14H8.E7-VH | EVQLQESGAELVKPGASVKLSCKASGYTFTNYWINWLKQRPGQGLEWI |
| | | GNIYPGSTIINYNEKFKNKATLTVDTSSSTAYMQLSSLTSDDSAVYYC |
| | | ARRVVYLYFDSWGQGTTLTVSS |
| 132 | 14H8.E7-VH CDR1 | GYTFTNY |
| 133 | 14H8.E7-VH CDR2 | YPGSTI |
| 134 | 14H8.E7-VH CDR3 | RVVYLYFDS |
| 135 | Alpha-FLT3 mAb- | QVQLQQPGAELVKPGASLKLSCKSSGYTFTSYWMHWVRQRPGHGLEWI |
| | 4G8 (Synimmune) | GEIDPSDSYKDYNQKFKDKATLTVDRSSNTAYMHLSSLTSDDSAVYYC |
| | VH | ARAITTTPFDFWGQGTTLTVSS |
| 136 | Alpha-FLT3 mAb- | DIVLTQSPATLSVTPGDSVSLSCRASQSISNNLHWYQQKSHESPRLLI |
| | 4G8 (Synimmune) | KYASQSISGIPSRFSGSGSGTDFTLSINSVETEDFGVYFCQQSNTWPY |
| | VL | TFGGGTKLEIK |
| 137 | Alpha-FLT3 mAb- | EVQLVQSGAEVKKPGASVKVSCKASGYTFTSYYMHWVRQAPGQGLEWM |
| | EB10 | GIINPSGGSTSYAQKFQGRVTMTRDTSTSTVYMELSSLRSEDTAVYYC |
| | (ImClone/Lilly) | ARGVGAHDAFDIWGQGTTVTVSS |
| | VH | |
| 138 | Alpha-FLT3 mAb- | DVVMTQSPLSLPVTPGEPASISCRSSQSLLHSNGNNYLDWYLQKPGQS |
| | EB10 | PQLLIYLGSNRASGVPDRFSGSGSDTDFTLQISRVEAEDVGVYYCMQG |
| | (ImClone/Lilly) | THPAISFGQGTRLEIK |
| | VL | |
| 139 | Alpha-FLT3 mAb- | EVQLVQSGAEVKKPGSSVKVSCKASGGTFSSYAISWVRQAPGQGLEWM |
| | NC7 | GGIIPIFGTANYAQKFQGRVTITADKSTSTAYMELSSLRSEDTAVYYC |
| | (ImClone/Lilly) | ATFALFGFREQAFDIWGQGTTVTVSS |
| | VH | |
| 140 | Alpha-FLT3 mAb- | DIQMTQSPSSLSASVGDRVTITCRASQSISSYLNWYQQKPGKAPKLLI |
| | NC7 | YAASSLQSGVPSRFSGSGSGTDFTLTISSLQPEDLATYYCQQSYSTPF |
| | (ImClone/Lilly) | TFGPGTKVDIK |
| | VL | |
| 141 | Alpha-FLT3 mAb- | QVTLKESGPALVKPTETLTLTCTVSGFSFRNARMGVSWIRQPPGKALE |
| | FL23 (Amgen) | WLAHIFSNDEKSYSTSLKSRLTISKDTSKSQVVLTLTNMDPVDTATYF |
| | VH | CARMPEYSSGWSGAFDIWGQGTMVTVSS |
| 142 | Alpha-FLT3 mAb- | DIQMTQSPSSLSASVGDRVTITCRASQDIGYDLGWYQQKPGKAPKRLI |
| | FL23 (Amgen) | YAASTLQSGVPSRFSGSGSGTEFTLIISSLQPEDFATYYCLQHNSFPW |
| | VL | TFGQGTKVEIK |
| 143 | Alpha-FLT3 mAb- | QVTLKESGPTLVKPTETLTLTCTLSGFSLNNARMGVSWIRQPPGKCLE |
| | FL39 (Amgen) | WLAHIFSNDEKSYSTSLKNRLTISKDSSKTQVVLTMTNVDPVDTATYY |
| | VH | CARIVGYGSGWYGFFDYWGQGTLVTVSS |
| 144 | Alpha-FLT3 mAb- | DIQMTQSPSSLSASVGDRVTITCRASQGIRNDLGWYQQKPGKAPKRLI |
| | FL39 (Amgen) | YAASTLQSGVPSRFSGSGSGTEFTLTISSLQPEDFATYYCLQHNSYPL |
| | VL | TFGCGTKVEIK |
| 145 | Alpha-FLT3 mAb- | QVQLVESGGGVVQPGRSLRLSCAASGFTFSSYGMHWVRQAPGKGLEWV |
| | FL61 (Amgen) | AVISYDGSNEFYADSVKGRFTISRDNSKNTLYLQMNSLRAEDTAVYYC |
| | VH | ARGGEITMVRGVIGYYYYGMDVWGQGTTVTVSS |
| 146 | Alpha-FLT3 mAb- | DIQMTQSPSSLSASVGDRVTITCRASQSISSYLNWYQQKPGKAPKLLI |
| | FL61 (Amgen) | YAASSLQSGVPSRFSGSGSGTEFTLTISSLQPEDFATYYCLQHNSYPL |
| | VL | TFGGGTKVEIK |

What is claimed is:

1. An antigen-binding site that binds FLT3, comprising:
(a) a heavy chain variable domain (VH) comprising complementarity-determining region 1 (CDR1), complementarity-determining region 2 (CDR2), and complementarity-determining region 3 (CDR3) comprising the amino acid sequences of SEQ ID NOs: 11, 4, and 55, respectively; and
(b) a light chain variable domain (VL) comprising CDR1, CDR2, and CDR3 comprising the amino acid sequences of SEQ ID NOs: 6, 7, and 8, respectively.

2. The antigen-binding site of claim 1, wherein the CDR3 of the VH comprises the amino acid sequence of SEQ ID NO:5.

3. The antigen-binding site of claim 1, wherein the CDR3 of the VH comprises the amino acid sequence of SEQ ID NO:50.

4. The antigen-binding site of any one of claims 1-3, wherein the VH comprises an amino acid sequence at least 90% identical to SEQ ID NO:37, and the VL comprises an amino acid sequence at least 90% identical to SEQ ID NO:38.

5. The antigen-binding site of any one of claims 1-4, wherein the VH comprises the amino acid sequence of SEQ ID NO:53, and the VL comprises the amino acid sequence of SEQ ID NO:42.

6. The antigen-binding site of claim 5, wherein the VH and the VL comprise the amino acid sequences of SEQ ID NOs: 9 and 10; 13 and 10; 17 and 10; 9 and 22; 9 and 26; 9 and 30; 9 and 34; 37 and 38; 41 and 42; 45 and 42; or 49 and 42, respectively.

7. An antigen-binding site that binds FLT3, comprising:
(a) a VH comprising CDR1, CDR2, and CDR3 comprising the amino acid sequences of SEQ ID NOs: 59, 63, and 54, respectively; and
(b) a VL comprising CDR1, CDR2, and CDR3 comprising the amino acid sequences of SEQ ID NOs: 86, 66, and 67, respectively.

8. The antigen-binding site of claim 7, wherein the VH comprises CDR1, CDR2, and CDR3 of SEQ ID NO: 78, 63, 79, respectively, and the VL comprises CDR1, CDR2, and CDR3 of SEQ ID NO: 80, 66, 67, respectively.

9. The antigen-binding site of claim 7, wherein the VH comprises CDR1, CDR2, and CDR3 of SEQ ID NO: 62, 63, 64, respectively, and the VL comprises CDR1, CDR2, and CDR3 of SEQ ID NO: 65, 66, 67.

10. The antigen-binding site of claim 7, wherein the VH comprises an amino acid sequence at least 90% identical to SEQ ID NO:76, and the VL comprises an amino acid sequence at least 90% identical to SEQ ID NO:77.

11. The antigen-binding site of any one of claims 7-10, wherein the VH comprises the amino acid sequence of SEQ ID NO:29, and the VL comprises the amino acid sequence of SEQ ID NO:84.

12. The antigen-binding site of claim 11, wherein the VH and the VL comprise the amino acid sequences of SEQ ID NOs: 68 and 69; 72 and 73; or 76 and 77, respectively.

13. An antigen-binding site that binds FLT3, comprising:
(a) a VH comprising CDR1, CDR2, and CDR3 comprising the amino acid sequences of SEQ ID NOs: 87, 88, and 89, respectively; and
(b) a VL comprising CDR1, CDR2, and CDR3 comprising the amino acid sequences of SEQ ID NOs: 91, 92, and 93, respectively.

14. An antigen-binding site that binds FLT3, comprising:
(a) a VH comprising CDR1, CDR2, and CDR3 comprising the amino acid sequences of SEQ ID NOs: 97, 99, and 100, respectively; and
(b) a VL comprising CDR1, CDR2, and CDR3 comprising the amino acid sequences of SEQ ID NOs: 101, 102, and 103, respectively.

15. An antigen-binding site that binds FLT3, comprising:
(a) a VH comprising CDR1, CDR2, and CDR3 comprising the amino acid sequences of SEQ ID NOs: 87, 98, and 89, respectively; and
(b) a VL comprising CDR1, CDR2, and CDR3 comprising the amino acid sequences of SEQ ID NOs: 106, 92, and 93, respectively.

16. An antigen-binding site that binds FLT3, comprising:
(a) a VH comprising CDR1, CDR2, and CDR3 comprising the amino acid sequences of SEQ ID NOs: 109, 110, and 111, respectively; and
(b) a VL comprising CDR1, CDR2, and CDR3 comprising the amino acid sequences of SEQ ID NOs: 112, 113, and 114, respectively.

17. An antigen-binding site that binds FLT3, comprising:
(a) a VH comprising CDR1, CDR2, and CDR3 comprising the amino acid sequences of SEQ ID NOs: 117, 118, and 119, respectively; and
(b) a VL comprising CDR1, CDR2, and CDR3 comprising the amino acid sequences of SEQ ID NOs: 120, 121, and 122, respectively.

18. An antigen-binding site that binds FLT3, comprising:
(a) a VH comprising CDR1, CDR2, and CDR3 comprising the amino acid sequences of SEQ ID NOs: 87, 98, and 89, respectively; and
(b) a VL comprising CDR1, CDR2, and CDR3 comprising the amino acid sequences of SEQ ID NOs: 106, 92, and 93, respectively.

19. An antigen-binding site that binds FLT3, comprising:
(a) a VH comprising CDR1, CDR2, and CDR3 comprising the amino acid sequences of SEQ ID NOs: 62, 33, and 127, respectively; and
(b) a VL comprising CDR1, CDR2, and CDR3 comprising the amino acid sequences of SEQ ID NOs: 128, 129, and 130, respectively.

20. An antigen-binding site that binds FLT3, comprising:
(a) a VH comprising CDR1, CDR2, and CDR3 comprising the amino acid sequences of SEQ ID NOs: 132, 133, and 134, respectively; and
(b) a VL comprising CDR1, CDR2, and CDR3 comprising the amino acid sequences of SEQ ID NOs: 65, 66, and 46, respectively.

21. An antigen-binding site that competes with the antigen-binding site of any one of claims 13-15 and 18-19.

22. The antigen-binding site of any one of the preceding claims, wherein the antigen-binding site binds human FLT3 with a dissociation constant (K_{D}) smaller than or equal to 20 nM as measured by surface plasmon resonance (SPR).

23. The antigen-binding site of any one of claims 1-6, 13, 14, and 18, wherein the antigen-binding site binds human FLT3 with a K_{D} smaller than or equal to 10 nM as measured by SPR.

24. The antigen-binding site of any one of claims 1-6, wherein the antigen-binding site binds a human FLT3 variant comprising the amino acid sequence of SEQ ID NO:25.

25. The antigen-binding site of any one of claims 1-6, wherein the antigen-binding site binds a human FLT3 variant comprising the amino acid sequence of SEQ ID NO:18.

26. The antigen-binding site of any one of claims 1-16 and 18-25, wherein the antigen-binding site binds cynomolgus FLT3.

27. The antigen-binding site of any one of claims 1-15 and 17-26, wherein the antigen-binding site does not compete with FLT3L for binding FLT3.

28. The antigen-binding site of any one of the preceding claims, wherein the antigen-binding site is present as a single-chain fragment variable (scFv).

29. The antigen-binding site of claim 28, wherein the scFv comprises an amino acid sequence selected from SEQ ID NOs: 3, 12, 15, 16, 19, 20, 23, 24, 27, 28, 31, 32, 35, 36, 39, 40, 43, 44, 47, 48, 51, 52, 70, 71, 74, 75, 81, and 82.

30. A protein comprising the antigen-binding site of any one of the preceding claims.

31. The protein of claim 30, further comprising an antibody heavy chain constant region.

32. The protein of claim 31, wherein the antibody heavy chain constant region is a human IgG heavy chain constant region.

33. The protein of claim 32, wherein the antibody heavy chain constant region is a human IgG1 heavy chain constant region.

34. The protein of claim 32 or 33, wherein each polypeptide chain of the antibody heavy chain constant region comprises an amino acid sequence at least 90% identical to SEQ ID NO:21.

35. The protein of any one of claims 32-34, wherein at least one polypeptide chain of the antibody heavy chain constant region comprises one or more mutations, relative to SEQ ID NO:21, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system.

36. The protein of any one of claims 32-35, wherein at least one polypeptide chain of the antibody heavy chain constant region comprises one or more mutations, relative to SEQ ID NO:21, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E, numbered according to the EU numbering system.

37. The protein of any one of claims 32-36, wherein one polypeptide chain of the antibody heavy chain constant region comprises one or more mutations, relative to SEQ ID NO:21, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to SEQ ID NO:21, at one or more positions selected from Q347, Y349, L351, S354, E356, E357, S364, T366, L368, K370, N390, K392, T394, D399, D401, F405, Y407, K409, T411, and K439, numbered according to the EU numbering system.

38. The protein of claim 37, wherein one polypeptide chain of the antibody heavy chain constant region comprises K360E and K409W substitutions relative to SEQ ID NO:21; and the other polypeptide chain of the antibody heavy chain constant region comprises Q347R, D399V and F405T substitutions relative to SEQ ID NO:21, numbered according to the EU numbering system.

39. The protein of claim 37 or 38, wherein one polypeptide chain of the antibody heavy chain constant region comprises a Y349C substitution relative to SEQ ID NO:21; and the other polypeptide chain of the antibody heavy chain constant region comprises an S354C substitution relative to SEQ ID NO:21, numbered according to the EU numbering system.

40. An antibody-drug conjugate comprising the protein of any one of claims 30-39 and a drug moiety.

41. The antibody-drug conjugate of claim 40, wherein the drug moiety is selected from the group consisting of auristatin, N-acetyl-γ calicheamicin, maytansinoid, pyrrolobenzodiazepine, and SN-38.

42. An immunocytokine comprising the antigen-binding site of any one of claims 1-29 and a cytokine.

43. The immunocytokine of claim 42, wherein the cytokine is selected from the group consisting of IL-2, IL-4, IL-10, IL-12, IL-15, TNF, and IFNα.

44. A bispecific T-cell engager comprising the antigen-binding site of any one of claims 1-29 and an antigen-binding site that binds CD3.

45. A chimeric antigen receptor (CAR) comprising:
(a) the antigen-binding site of any one of claims 1-29;
(b) a transmembrane domain; and
(c) an intracellular signaling domain.

46. The CAR of claim 45, wherein the transmembrane domain is selected from the transmembrane regions of the alpha, beta or zeta chain of the T-cell receptor, CD28, CD3 epsilon, CD45, CD4, CD5, CD8, CD9, CD16, CD22, FLT3, CD37, CD64, CD80, CD86, CD134, CD137, CD152, and CD154.

47. The CAR of claim 45 or 46, wherein the intracellular signaling domain comprises a primary signaling domain comprising a functional signaling domain of CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12.

48. The CAR of any one of claims 45-47, wherein the intracellular signaling domain further comprises a costimulatory signaling domain comprising a functional signaling domain of a costimulatory receptor.

49. The CAR of claim 48, wherein the costimulatory receptor is selected from the group consisting of OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

50. An isolated nucleic acid encoding the CAR of any one of claims 45-49.

51. An expression vector comprising the isolated nucleic acid of claim 50.

52. An immune effector cell comprising the nucleic acid of claim 50 or the expression vector of claim 51.

53. An immune effector cell expressing the CAR of any one of claims 45-49.

54. The immune effector cell of claim 52 or 53, wherein the immune effector cell is a T cell.

55. The immune effector cell of claim 54, wherein the T cell is a CD8⁺ T cell, a CD4⁺ T cell, or an NKT cell.

56. The immune effector cell of claim 52 or 53, wherein the immune effector cell is an NK cell.

57. A pharmaceutical composition comprising the protein of any one of claims 30-39, the antibody-drug conjugate of claim 40 or 41, the immunocytokine of claim 42 or 43, the bispecific T-cell engager of claim 44, or the immune effector cell of any one of claims 52-56; and a pharmaceutically acceptable carrier.

58. A method of treating cancer, the method comprising administering to a subject in need thereof an effective amount of the protein of any one of claims 30-39, the antibody-drug conjugate of claim 40 or 41, the immunocytokine of claim 42 or 43, the bispecific T-cell engager of claim 44, the immune effector cell of any one of claims 52-56, or the pharmaceutical composition of claim 57.

59. The method of claim 58, wherein the cancer is a hematologic malignancy.

60. The method of claim 59, wherein the hematologic malignancy is leukemia.

61. The method of claim 59 or 60, wherein the cancer is selected from the group consisting of acute myeloid leukemia (AML), acute lymphoblastic leukemia (ALL), myelodysplasia, acute T-lymphoblastic leukemia, and acute promyelocytic leukemia.

62. The method of any one of claims 58-61, wherein the cancer expresses FLT3.
